SEQUENCE LISTING (42 sequences)

SEQ ID NO: 1 - DNA, 117 bp, Homo sapiens
  source 1..117: mol_type = genomic DNA
attgaagttatgtatcctcctccttacctagacaatgagaagagcaatggaaccattatccatgtgaaagggaaacacctttgtccaagtcccctatttcccggaccttctaagccc

SEQ ID NO: 2 - protein, 39 aa, Homo sapiens
IEVMYPPPYLDNEKSNGTIIHVKGKHLCPSPLFPGPSKP

SEQ ID NO: 3 - DNA, 135 bp, Homo sapiens
  source 1..135: mol_type = genomic DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 4 - protein, 45 aa, Homo sapiens
TTTPAPRPPTPAPTIASQPLSLRPEACRPAAGGAVHTRGLDFACD

SEQ ID NO: 5 - DNA, 81 bp, Homo sapiens
  source 1..81: mol_type = genomic DNA
ttttgggtgctggtggtggttgggggagtcctggcttgctatagcttgctagtaacagtggcctttattattttctgggtg

SEQ ID NO: 6 - protein, 27 aa, Homo sapiens
FWVLVVVGGVLACYSLLVTVAFIIFWV

SEQ ID NO: 7 - DNA, 63 bp, Homo sapiens
  source 1..63: mol_type = genomic DNA
atctacatctgggcgcccttggccgggacttgtggggtccttctcctgtcactggttatcacc

SEQ ID NO: 8 - protein, 21 aa, Homo sapiens
IYIWAPLAGTCGVLLLSLVIT

SEQ ID NO: 9 - DNA, 63 bp, Homo sapiens
  source 1..63: mol_type = genomic DNA
ccattttttttctgctgcttcatcgctgtagccatgggaatccgtttcattattatggtagca

SEQ ID NO: 10 - protein, 21 aa, Homo sapiens
PFFFCCFIAVAMGIRFIIMVA

SEQ ID NO: 11 - DNA, 123 bp, Homo sapiens
  source 1..123: mol_type = genomic DNA
aggagtaagaggagcaggctcctgcacagtgactacatgaacatgactccccgccgccccgggcccacccgcaagcattaccagccctatgccccaccacgcgacttcgcagcctatcgctcg

SEQ ID NO: 12 - protein, 41 aa, Homo sapiens
RSKRSRLLHSDYMNMTPRRPGPTRKHYQPYAPPRDFAAYRS

SEQ ID NO: 13 - DNA, 126 bp, Homo sapiens
  source 1..126: mol_type = genomic DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 14 - protein, 42 aa, Homo sapiens
KRGRKKLLYIFKQPFMRPVQTTQEEDGCSCRFPEEEEGGCEL

SEQ ID NO: 15 - DNA, 360 bp, Homo sapiens
  source 1..360: mol_type = genomic DNA
tggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattcc

SEQ ID NO: 16 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 17 - DNA, 339 bp, Homo sapiens
  source 1..339: mol_type = genomic DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 18 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 19 - DNA, 114 bp, Homo sapiens
  source 1..114: mol_type = genomic DNA
caagtgcgaaaggcagctataaccagctatgagaaatcagatggtgtttacacgggcctgagcaccaggaaccaggagacttacgagactctgaagcatgagaaaccaccacag

SEQ ID NO: 20 - protein, 38 aa, Homo sapiens
QVRKAAITSYEKSDGVYTGLSTRNQETYETLKHEKPPQ

SEQ ID NO: 21 - DNA, 66 bp, Homo sapiens
  source 1..66: mol_type = genomic DNA
atggattttcaggtgcagattttcagcttcctgctaatcagtgcctcagtcataatgtccagagga

SEQ ID NO: 22 - protein, 22 aa, Homo sapiens
MDFQVQIFSFLLISASVIMSRG

SEQ ID NO: 23 - DNA, 1704 bp, Homo sapiens
  source 1..1704: mol_type = genomic DNA
atggattttcaggtacaaattttctcattccttctcatttcagcatccgtaataatgtccagaggcgacatccaaatgactcaatcaccaagttccttgtccgcgtccgttggggatagagtaaccataacctgtagggcatcacaagatgtgaatacggccgtagcgtggtatcaacagaaaccaggaaaggctccaaaactccttatttattccgcgagttttctttacagcggagtcccgagtagattctctggttctcgctctggcactgattttactctgaccataagttcactgcaacctgaagactttgcgacctattactgtcaacagcactacacaacgcctcccacttttggacaaggtactaaagtagagattaaacggaccggatccacaagtgggagcgggaagcctggttctggcgaaggatccgaagttcagctggttgaatccggcggtgggttggtgcaacccggggggagcctgcgcctcagctgtgccgcgagtgggtttaacataaaagatacctacattcactgggtgcgccaagctccgggcaaaggacttgaatgggtcgcgaggatctacccgaccaacggttacacaagatatgcggactccgtaaaagggcgattcacgatatccgctgacacatccaagaacacggcgtacttgcaaatgaattctcttagggccgaggacaccgcagtttattactgtagtcgctggggaggtgatggattttatgcgatggacgtttgggggcaagggacgctggtcacggtttccagtgctgcaaccacaacgccagcaccaagacctcccacacccgctcctaccatcgcttcacaacccctttctctgcgaccagaggcgtgtagacccgccgctgggggcgccgttcacacgaggggcctggacttcgcgtgcgacccctttttcttctgctgctttatagctgtggcgatgggaattcgatttataattatggtggcatggagacggaagcggaaggagaaacagtccgagactagcccgaaggagttcttgaccatttatgaagacgtaaaagatttgaagacccggcgcaatcacgaacaagaacaaacgtttccaggaggcggtagtactatatactcaatgattcaaagtcaatcttcagcaccgacttctcaagaacccgcatatactctctatagcctgattcaaccctcacggaagtcagggagcaggaaaaggaaccattcaccgagttttaattccacgatttacgaggtgataggcaagagccagcctaaggcccagaacccggcaagattgtcccgaaaggaactcgaaaactttgatgtgtactctagggtgaaattcagtcggtcagcagatgcccctgcatatcaacaaggacaaaaccaactgtataacgagctcaatcttggtcgacgcgaggaatacgacgtacttgacaagagaagaggaagagaccccgagatggggggcaagccgcagcgaagaaagaatccacaagaagggctctacaatgaactgcagaaggacaaaatggccgaagcctattccgagatcggaatgaagggtgagcgaaggcgaggaaaggggcacgacggcctttaccagggtctgtcaaccgctacgaaagacacgtacgacgccctgcacatgcaagcgctcccaccacgatag

SEQ ID NO: 24 - protein, 567 aa, Homo sapiens
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSAATTTPAPRPPTPAPTIASQPLSLRPEACRPAAGGAVHTRGLDFACDPFFFCCFIAVAMGIRFIIMVAWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 25 - DNA, 1470 bp, Homo sapiens
  source 1..1470: mol_type = genomic DNA
atggacttccaagtgcagatattctcctttcttcttatttccgcatccgttataatgagcaggggggatatacaaatgactcagtctccgtcctctctgagtgcatccgtgggcgaccgagtaactatcacgtgccgggcatcccaagatgtaaacacagccgtagcttggtaccaacaaaaaccaggtaaagctcctaaattgcttatttactctgcaagtttcttgtacagtggggtgccgtcccggtttagtggctccagaagcggcactgatttcactctgacaatatcttctcttcaacccgaggattttgctacatattattgccaacaacattatacgactccccctactttcggccaagggactaaagtcgagatcaaacgaaccggttccacgtccgggtcaggtaaacctggttcaggagaaggatcagaggtgcaattggtcgagagtggcgggggattggtacaaccaggtggcagtcttaggctgtcttgtgcagcctcaggctttaacattaaggacacgtacatacactgggtccgacaagcaccgggcaagggtctggaatgggtggcacggatttatcctacaaacggatatactcgatacgcggattcagtcaaggggcgctttaccataagtgccgacactagcaagaacactgcctatcttcagatgaattcattgagggcggaagacactgcggtatactattgctctagatggggaggagacggcttttatgcaatggatgtgtggggtcagggaaccctcgttaccgtatcttctgctgcaaccaccacccccgccccccgaccaccgacaccagcaccaacaatcgcatcccagcctttgtcattgagaccagaggcgtgtagacccgcagctggaggcgcagtccatacgcgggggctggattttgcctgcgacatatatatatgggctcctctcgcgggtacgtgcggtgttttgctcctgtcactcgtgataacaaagcgaggccggaaaaaattgctctatatcttcaaacagccgttcatgcgaccggtgcagacaacacaagaagaagacggctgcagctgcagattccctgaggaggaagaaggtgggtgtgaattgagggttaaattctcccgaagcgcggatgcaccggcgtatcagcagggccaaaatcaactctacaacgagctcaacctggggcggcgggaagagtacgacgtactggacaagcgccgaggcagagaccctgagatggggggcaagcctcaaaggcgaaagaaccctcaggaggggctttacaacgagctccaaaaggacaagatggcggaggcctattcagaaatcggcatgaaaggcgaacgaaggagaggtaaaggacacgacgggctgtatcagggtttgagtactgcaacaaaggatacctacgacgctctccacatgcaagcgctgcccccaaggtag

SEQ ID NO: 26 - protein, 489 aa, Homo sapiens
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSAATTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 27 - DNA, 1464 bp, Homo sapiens
  source 1..1464: mol_type = genomic DNA
atggattttcaggtacagattttctcttttcttcttataagcgcgtctgtgatcatgagtcggggcgatatacagatgacccaaagcccttcctcactgtcagcgagcgtaggagatagggtgaccatcacatgcagggcgagccaagatgtgaacacagccgtagcttggtatcagcaaaagccaggcaaggctcccaaactgctgatatattctgctagctttctgtattccggtgtacccagcaggttcagcggctccagaagtggaaccgacttcactctgacaatcagtagccttcaaccagaagatttcgcaacgtactactgtcaacaacattacacgacgcccccaaccttcgggcaaggaacgaaagttgagataaaaaggaccggcagcacctccggaagcgggaagccaggatccggggagggttccgaggtgcaactcgtcgagtcaggtggcggtctcgtgcaaccaggaggctccctgcggctctcttgtgcggctagtggatttaatataaaagatacctatattcactgggtgcgccaagcacctggaaaagggctggagtgggtcgccaggatatatccgacaaatggatacacacggtatgcggacagtgttaaaggcaggttcacaattagcgcagacacgagcaagaatacagcctatcttcagatgaattctctcagggctgaagatactgcagtctactattgctctaggtggggtggtgacggcttttacgctatggatgtctgggggcagggcactctggttactgtcagctctgcgatagaagtcatgtaccctccgccgtatcttgacaatgagaagtctaatgggacaatcatacacgtgaaaggcaagcacttgtgcccctctcccctgttccccggccctagtaaaccgttctgggtgctcgtagtggtcggtggagttcttgcctgttatagtttgttggtaaccgtcgcgtttataatattctgggtccggtccaagagaagccgcctcctgcattccgattacatgaacatgaccccacggaggcccggccctacacggaagcattaccagccatacgctccgcctcgagattttgctgcttataggtcacgagtaaagtttagtagatccgctgacgcccctgcctaccagcagggtcagaatcagctctacaatgaactgaacttgggcaggcgagaagagtatgacgttctcgacaagcgaagggggagagatcccgaaatgggtggtaaaccacaaagacgcaagaatcctcaggaaggattgtacaacgagctccaaaaggataagatggcggaagcatattccgaaattgggatgaagggcgagcgaaggcggggcaaaggacacgacggcctttatcaaggactgtctacggctactaaagacacttatgatgcgctgcacatgcaagcattgccgccgagatag

SEQ ID NO: 28 - protein, 487 aa, Homo sapiens
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 29 - DNA, 1701 bp, Homo sapiens
  source 1..1701: mol_type = genomic DNA
atggatttccaagtccaaatctttagttttttgcttatttcagcctccgtcattatgagtagaggggatatacaaatgacccagtcccctagtagcctgagcgcaagcgttggagaccgggtaaccataacatgtagggctagccaggacgttaatacagccgttgcgtggtatcagcaaaaaccgggcaaagcacctaaactcctgatctatagcgcctcattcctgtatagcggcgtccctagtcgattctcaggaagtagatctggtactgacttcacgttgaccatatcctcactccaacccgaggacttcgcaacatattattgccagcagcactataccacgccgccgacgttcgggcaagggaccaaagtcgaaattaagagaacagggtctacgagtggcagtggtaaacccggttccggcgaggggtccgaagtgcaattggtggaatctggagggggtctcgtacaaccggggggctcccttagactcagttgtgccgcgagcggcttcaatattaaggacacttatatccattgggttcggcaagctcccggcaagggtcttgaatgggtagcacgcatatacccgacaaatggatacacgcggtacgcggatagtgtcaagggtaggtttaccatatctgcggacacgtccaaaaacaccgcctaccttcagatgaattccctccgcgctgaagacactgcggtgtattactgcagccgctggggcggggatgggttttacgccatggatgtgtggggtcagggtacactcgtaactgtgagcagcgccaccacgacgcccgcaccgaggccaccgactccagcacccactatcgcttctcaacctctgtcactgcgccctgaagcctgtcggcctgcggccgggggagcggttcatacgcgggggctggatttcgcttgcgacatttatatatgggcacccctcgcgggtacatgtggcgtcctcttgctgagtctcgtaattacgtggagacgaaagaggaaagagaagcagtctgaaactagccctaaggagttccttactatatatgaggatgttaaagatctgaaaacccgaagaaaccatgaacaggaacaaactttccctggcgggggaagtacgatttacagcatgatccaatctcagtcaagtgcgccaaccagtcaagaacctgcgtatacattgtattccctcattcagccatctaggaaaagcggttcacgcaaaaggaaccatagcccttctttcaattcaaccatctatgaagttattggtaaaagtcaacctaaagcgcaaaatccggcgcgacttagccgcaaagaactcgaaaacttcgatgtatacagtcgggtaaaattctctcgcagtgccgatgcgcctgcctaccagcagggacagaatcagttgtacaacgaactgaacctcggccgaagagaggagtatgatgttcttgataagcggaggggcagagatcccgagatgggtgggaagccacaaagacgaaaaaatcctcaggagggactgtacaatgaacttcaaaaagacaagatggctgaagcctactctgagattgggatgaaaggtgagcgccgaaggggaaagggccacgacggcttgtatcaaggactgtccaccgccactaaagatacgtacgatgccttgcatatgcaagcccttcctccacgctag

SEQ ID NO: 30 - protein, 566 aa, Homo sapiens
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSATTTPAPRPPTPAPTIASQPLSLRPEACRPAAGGAVHTRGLDFACDIYIWAPLAGTCGVLLLSLVITWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 31 - DNA, 1479 bp, Homo sapiens
  source 1..1479: mol_type = genomic DNA
atggactttcaagtccagattttctccttccttctcattagcgccagcgtaataatgagtcgaggtgacattcaaatgacacagagtcccagttccctcagtgcttccgtaggcgacagagttactattacgtgtagggcttcacaagatgttaatacggccgtggcttggtaccagcaaaaacctggaaaggctccaaaattgcttatatactcagcttcattcctctactccggggtcccgtcaaggttctctggttccaggtcaggcacagatttcacactcacaatctcctctctgcagccggaagactttgcgacatactattgtcagcaacattatacgaccccgcccactttcggacaaggtactaaggttgaaatcaagcgaacgggttcaacaagcggctcaggaaaaccagggtccggagaaggttctgaggtccagctggtcgagtcaggcggtggtctggtccaaccgggcggctcacttagactgagctgcgcagcgtctgggtttaatataaaggacacctatatacattgggtaaggcaagcgcccgggaaaggactggagtgggttgcaaggatttacccaacgaatggttatacacgctatgctgatagcgtaaaaggtcggtttacaatatctgctgacaccagcaagaatacagcgtaccttcaaatgaactctttgcgggccgaggatactgctgtttattactgctctcgctggggcggtgacggattttatgccatggacgtatggggacaggggactcttgtcacagtttccagtgccgctaccactacccccgcgccacgaccaccaactccagcacccactatagcatctcagccactgtccctgaggcccgaagcgtgtcgacccgcggcaggtggcgcagtgcatactcgcggattggatttcgcttgtgatccctttttcttttgttgctttatcgccgtggcgatgggtattcgatttataattatggtggcgtggcggcggaagagaaaagagaagcaaagcgaaacttctccaaaggagttcttgactatatatgaagacgtgaaggatctcaagacgcgccgcaatcatgagcaggagcagactttccctgggggcgggagcaccatatattcaatgatccaaagtcagtctagcgcacccacctcacaagagccggcctataccctgtactccctcatccagcctagcaggaaatctggaagccgaaagcgaaatcatagtccgagtttcaacagtacgatatatgaagtaataggtaaatcacaacccaaagcccagaatccagctcgcctttctcggaaggagctggagaattttgacgtctattcccaagtgcggaaagccgcaattacctcctacgaaaaaagcgatggtgtttacacgggcttgagcacacggaaccaggaaacttatgagaccttgaaacatgaaaagccaccgcaatag

SEQ ID NO: 32 - protein, 493 aa, Homo sapiens
  VARIANT 493: note = X can be any amino acid
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSAATTTPAPRPPTPAPTIASQPLSLRPEACRPAAGGAVHTRGLDFACDPFFFCCFIAVAMGIRFIIMVAWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSQVRKAAITSYEKSDGVYTGLSTRNQETYETLKHEKPPQX

SEQ ID NO: 33 - DNA, 1245 bp, Homo sapiens
  source 1..1245: mol_type = genomic DNA
atggatttccaggttcagattttctccttcctgttgatcagtgcgagtgtgattatgtctcgaggtgatatacagatgacgcaaagtccatcctcactgagtgcaagcgtgggtgacagggttactatcacttgtagggcaagccaggacgtgaatactgcagtcgcttggtaccagcagaagcctggaaaagcgccaaagctgctgatttatagcgctagttttctgtatagcggtgtaccgtcacggttctctgggagccggtctggaacggattttacccttaccatttcttcactccaaccagaagattttgccacttattattgccaacagcattacaccactcccccgacattcggacaagggacgaaagtagagatcaaacgcacgggctccacaagtggaagtgggaaacctggctccggtgagggatcagaggttcagctggtggaatccgggggcggtcttgtacagccgggtggatcacttaggttgagctgcgctgcatcaggattcaatatcaaggacacatacatccactgggtgcgccaagctccggggaagggtttggaatgggttgcacgcatttatcccacgaatggctacactcggtatgctgactctgtgaaaggacggtttactatctccgccgacacgagtaagaatacagcgtatcttcagatgaactctctcagggcagaggacacagcagtgtactattgcagcaggtggggaggggatggcttctacgctatggacgtatggggccaagggacgttggtaaccgtgtcttccgccgcaactaccacgccagcgcctcgaccacccactcccgcacccacaattgcgagtcaaccgctgtcactgagaccagaagcttgcagacccgccgcaggtggcgcggtacatactcgcgggctggacttcgcctgtgatatctatatctgggctcctttggctggcacgtgcggcgtgcttctcctttcactcgtgatcacaaaaaggggacggaagaagctcttgtatattttcaaacagccgtttatgcgcccggtacagactacgcaagaagaagacggttgctcctgcagatttcctgaggaagaggaaggcggctgcgagttgcaggttaggaaagccgcgataactagctacgagaagagtgatggggtatatacgggcctctccacaaggaaccaggaaacatatgagactcttaaacatgaaaaacctccccagtag

SEQ ID NO: 34 - protein, 414 aa, Homo sapiens
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSAATTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELQVRKAAITSYEKSDGVYTGLSTRNQETYETLKHEKPPQ

SEQ ID NO: 35 - DNA, 1239 bp, Homo sapiens
  source 1..1239: mol_type = genomic DNA
atggatttccaggttcagatattcagttttttgctcatttccgccagtgtcatcatgagcaggggcgatattcagatgacccagtctccaagttctctttcagcgtcagtaggagatcgagtaactataacttgcagagcttcacaggatgtcaacacggccgtagcctggtatcaacaaaaaccagggaaggcgccgaaacttctcatttacagtgcatccttcctttacagcggagtgccttcccgctttagcgggtcacgcagtggtactgacttcacgcttacaatatctagtctccagcctgaagattttgcaacgtactactgccaacaacactatactactccgcccacattcggccagggcacaaaagtagagataaagcgcacgggtagcacgtcaggttcaggcaaaccaggttccggagaaggcagtgaagttcagctggtcgaaagcgggggcggactcgtgcaacccggcggcagtctcagactttcatgcgccgcgagtggtttcaatatcaaagatacctacatacactgggttaggcaggccccaggaaaaggcctcgaatgggttgctcgaatctacccaaccaacggatacacaaggtacgcagactctgtgaaaggcagatttacgatctctgcagacacgtccaagaacaccgcttaccttcaaatgaactcactccgggccgaagatacagccgtatattattgcagccgatggggaggggacgggttctacgcaatggatgtatggggtcagggcaccttggtaacagtgtcctctgcaatcgaggtcatgtacccgcccccttacctggataatgagaaatcaaatggtaccatcattcatgtgaaggggaagcacctttgcccaagtccgctctttccagggccctccaagccgttttgggtccttgttgtcgttggtggagtgctggcttgttacagcttgcttgtaacggtagcttttataatattctgggtgagatctaagcggtctcgcctcctccactctgactacatgaatatgactccccgccgcccgggcccaacgcgcaagcattaccagccgtacgcaccacctagggactttgccgcttatcggtcacaagtaagaaaagcagcaattacatcctatgagaagtccgatggggtttatacaggtctcagtacaaggaatcaagaaacgtacgagactttgaagcacgagaagccaccgcagtag

SEQ ID NO: 36 - protein, 413 aa, Homo sapiens
  VARIANT 413: note = X can be any amino acid
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQX

SEQ ID NO: 37 - DNA, 1476 bp, Homo sapiens
  source 1..1476: mol_type = genomic DNA
atggactttcaagtacaaatttttagcttcctcttgatctcagcaagtgtaataatgtcccgaggtgacatacagatgacacaaagtccctcctcacttagcgcttctgtgggagaccgagtaacaataacttgcagagcgtcccaagatgttaacacagccgtagcatggtatcagcaaaagcccggtaaagcgccgaaactgctcatctattcagcgtccttcctctattcaggggtgcccagcagattctctggtagccgctccgggactgactttactttgactattagttctctccagccggaagacttcgccacttactattgccaacagcactacacaacccccccgacttttgggcagggtactaaagttgaaatcaaacgcacgggctccaccagtggatccgggaaacctggaagcggcgaaggttccgaagttcagctcgttgaatccgggggtggccttgtgcaacctggaggatcccttcggttgagttgcgcagcctcaggatttaacattaaagacacgtatatccattgggtgaggcaggccccagggaagggtttggagtgggttgccagaatataccctacaaacggttatacccgctacgcggacagcgtcaaaggccggttcactatcagcgcggatacaagcaagaatactgcatatctccagatgaattcactgcgggctgaagacacagccgtttattactgctctcgatggggaggagatggtttctatgcgatggacgtctggggccaggggacgcttgttacagtctcttcagcaacaactactccagcccctcgacctccaacgccagcacccaccatagcttcacaacccctttctctgcgcccggaggcatgtagaccagccgccggaggagcggtgcacacacgggggctcgactttgcatgcgacatttatatatgggcccctcttgcaggtacctgcggtgttttgttgttgtcattggtcataacatggagaagaaaacgaaaggaaaaacagtcagagacatctccaaaggagttcctcactatctatgaggatgtgaaggatttgaagacgcgcagaaaccatgagcaggagcaaactttccccggtggtggatctacgatttactccatgattcaatctcaatcttccgcgcctacgtcccaggaaccggcatatactctttattctctcattcagcctagccggaagagtggctccagaaaacgcaatcattccccgtcatttaactcaaccatctatgaggtgattggtaagtctcagccgaaagcacaaaatcccgctaggctgagtcggaaagagcttgagaacttcgacgtgtatagtcaggttcgaaaggcagcaatcaccagctacgaaaaaagcgacggtgtctatactggcttgtccactagaaaccaggaaacgtatgaaacacttaagcatgagaaacccccacaatag

SEQ ID NO: 38 - protein, 492 aa, Homo sapiens
  VARIANT 492: note = X can be any amino acid
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSATTTPAPRPPTPAPTIASQPLSLRPEACRPAAGGAVHTRGLDFACDIYIWAPLAGTCGVLLLSLVITWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSQVRKAAITSYEKSDGVYTGLSTRNQETYETLKHEKPPQX

SEQ ID NO: 39 - DNA, 1002 bp, Homo sapiens
  source 1..1002: mol_type = genomic DNA
atggagggtatttcaatttatacctccgacaattacacggaggaaatgggaagtggagattacgactccatgaaagagccatgctttagggaagagaacgccaactttaataagatctttctcccgacaatttattctattatcttcctcaccggcattgtcggaaatggtcttgttattcttgttatgggctatcaaaagaaacttcgatctatgactgataagtacaggcttcacctcagtgtcgccgatctgttgtttgtaataactcttccgttttgggcggtagatgccgttgctaactggtacttcggtaacttcctttgtaaagctgtacatgtcatttacacagttaatctgtatagcagcgtgctgatactggcgtttatttcactggaccgctacctcgccatcgttcacgccacgaatagtcagcgaccgcggaagctcttggccgagaaggttgtgtacgtgggcgtgtggattcccgctctgctcttgacaattcccgacttcatatttgccaatgtgagcgaagccgatgatcgctacatctgtgatagattctatccgaatgatttgtgggtagtagtttttcaattccaacacatcatggtgggcctgatcttgccgggaatcgtaatcctgagttgttactgcattattatctctaagttgtcccactcaaagggccatcagaagcgcaaggcgttgaagacgactgttatactcatattggcattctttgcctgttggctgccttactatattgggatatctatagattctttcatactcctggagatcataaagcagggatgtgaatttgaaaatactgtacataagtggatttccataactgaagcgcttgcgttctttcattgttgtctgaatccaattctctatgcgtttctgggggcaaaatttaaaacctcagcgcaacatgcactgaccagtgtatcacggggctctagccttaaaatactttccaaaggcaagtaa

SEQ ID NO: 40 - protein, 334 aa, Homo sapiens
  VARIANT 334: note = X can be any amino acid
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKX

SEQ ID NO: 41 - DNA, 738 bp, Homo sapiens
  source 1..738: mol_type = genomic DNA
gatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctttattctggagtcccttctcgcttctctggatctagatctgggacggatttcactctgaccatcagcagtctgcagccggaagacttcgcaacttattactgtcagcaacattatactactcctcccacgttcggacagggtaccaaggtggagatcaaacgcactgggtctacatctggatctgggaagccgggttctggtgagggttctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggacgtgtggggtcaaggaaccctggtcaccgtctcctcggcggcc

SEQ ID NO: 42 - protein, 246 aa, Homo sapiens
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSAA

(19) United States
(12) Patent Application Publication
(10) Pub. No.: US 2024/0335472 A1
(43) Pub. Date: Oct. 10, 2024

(54) ANTI-HER2 CAR NK CELLS, METHODS OF THEIR PRODUCTION AND USES THEREOF

(71) Applicant: Gamida Cell Ltd., Ramat Gan (IL)

(72) Inventors: Yona GEFFEN, Jerusalem (IL); Aviad PATO, Jerusalem (IL); Julia RIFMAN, Jerusalem (IL)

(21) Appl. No.: 18/682,699

(22) PCT Filed: Aug. 5, 2022
(86) PCT No.: PCT/US2022/039587

§ 371 Date: Feb. 9, 2024

Related U.S. Application Data
(60) Provisional application No. 63/231,359, filed on Aug. 10, 2021.

(51) Int. Cl. A61K 35/17; A61K 39/00; C07K 14/725; C07K 16/32; C12N 5/0783; C12N 15/85

(52) U.S. Cl. CPC A61K 35/17; A61K 39/001106; A61K 39/4613; A61K 39/4631; A61K 39/464406; C07K 14/7051; C07K 16/32; C12N 5/0646; C12N 15/85; A61K 2039/5156; A61K 2239/13; A61K 2239/17; A61K 2239/21; A61K 2239/59; C07K 2317/622; C07K 2319/02; C07K 2319/03; C12N 2500/38; C12N 2501/2315; C12N 2502/1114; C12N 2510/00

(57) ABSTRACT

A composition and method of ex vivo producing natural killer (NK) cells expressing a chimeric antigen receptor (CAR) or a transgenic T cell receptor (tg-TCR) capable of binding HER2 is disclosed. The method comprising: (a) expanding a population of NK cells by a method comprising: (i) culturing the population of NK cells under conditions allowing for cell proliferation, wherein the conditions comprise providing an effective amount of nutrients, serum, IL-15 and nicotinamide; and (ii) supplementing the population of NK cells with an effective amount of fresh nutrients, serum, IL-15 and nicotinamide 5-10 days following step (i) to produce expanded NK cells; so as to obtain an ex vivo expanded population of NK cells, and (b) upregulating expression of a CAR or a tg-TCR capable of binding HER2 in the ex vivo expanded population of NK cells.

### CROSS-REFERENCE TO RELATED APPLICATIONS

This application claims priority to, and the benefit of, U.S. Provisional Application No. 63/231,359, filed Aug. 10, 2021, the contents of which are incorporated herein by reference in their entireties.

### FIELD AND BACKGROUND

Human epidermal growth factor receptor 2 (HER2), also known as ErbB2, is a member of the epidermal growth factor receptor (EGFR) family. HER2 leads to activation of the EGFR signaling pathway by forming a heterodimer with three other members of the EGFR family. The activation of the EGFR signaling pathway is usually associated with the abnormal proliferation of cells and tumorigenesis, accordingly, HER2 has become one of the therapeutic targets of various cancers (such as breast carcinoma, gastric carcinoma, ovarian carcinoma, lung carcinoma, bladder carcinoma, etc.).

NK cells are cytotoxic lymphocytes that constitute a significant component of the innate immune system. These cells have a variety of functions, especially the killing of tumor cells, virus-infected cells, cells undergoing oncogenic transformation, and other abnormal cells in a living body.

NK cells have drawn considerable attention in recent years as a promising tool for immunotherapy in patients with various refractory hematological malignancies and solid tumors, however, the full therapeutic potential of NK cell-based immunotherapy has yet to be realized. Accordingly, there is a need in the art for methods to culture and expand NK cells and transgenic NK CAR cells such that the resulting cell fractions display increased homing, retention, and proliferation activities upon in vivo infusion, while maintaining their killing activity.

The present disclosure relates to methods of generating and culturing transgenic natural killer CAR (NK) cells, selection of expanded transgenic NK CAR cell populations for administration to subjects in need thereof and the therapeutic use of suitable, ex-vivo expanded transgenic NK CAR cell fractions for transplantation in the clinical setting, for treatment of hematological malignancies and other (e.g. malignant) conditions. The present invention also envisions compositions and kits comprising the expanded NK CAR cell fractions.

According to an aspect of some embodiments of the present invention there is provided a method of ex vivo producing natural killer (NK) cells expressing a chimeric antigen receptor (CAR) or a transgenic T cell receptor (tg-TCR) capable of binding HER2, the method comprising:
(a) expanding a population of NK cells by a method comprising:
(i) culturing the population of NK cells under conditions allowing for cell proliferation, wherein the conditions comprise providing an effective amount of nutrients, serum, IL-15 and nicotinamide; and
(ii) supplementing the population of NK cells with an effective amount of fresh nutrients, serum, IL-15 and nicotinamide 5-10 days following step (i) to produce expanded NK cells;
so as to obtain an ex vivo expanded population of NK cells, and
(b) upregulating expression of a CAR or a tg-TCR capable of binding HER2 in the ex vivo expanded population of NK cells, thereby producing the NK cells expressing the CAR or the tg-TCR capable of binding the HER2.

According to an aspect of some embodiments of the present invention there is provided an isolated population of NK cells obtainable according to the method of some embodiments of the invention.

According to an aspect of some embodiments of the present invention there is provided a pharmaceutical composition comprising the isolated population of NK cells of some embodiments of the invention and a pharmaceutically active carrier.

According to an aspect of some embodiments of the present invention there is provided a method of treating a disease associated with expression of HER2 in a subject in need thereof, the method comprising administering to the subject a therapeutically effective amount of the isolated population of NK cells of some embodiments of the invention, thereby treating the subject.

According to an aspect of some embodiments of the present invention there is provided a therapeutically effective amount of the isolated population of NK cells of some embodiments of the invention for use in treating a disease associated with expression of HER2 in a subject in need thereof.

According to some embodiments of the invention, the population of NK cells is derived from cord blood, peripheral blood, bone marrow, CD34+ cells or iPSCs.

According to some embodiments of the invention, the population of NK cells is deprived of CD3⁺ cells.

According to some embodiments of the invention, the population of NK cells comprises CD3⁻CD56⁺ cells.

According to some embodiments of the invention, the effective amount of the nicotinamide comprises an amount between 1.0 mM to 10 mM.

According to some embodiments of the invention, expanding the population of NK cells is affected in the presence of feeder cells or a feeder layer.

According to some embodiments of the invention, the feeder cells comprise irradiated cells.

According to some embodiments of the invention, the feeder cells comprise T cells or PBMCs.

According to some embodiments of the invention, the conditions allowing for cell proliferation further comprise a CD3 agonist.

According to some embodiments of the invention, expanding the population of NK cells is affected for 14-16 days.

According to some embodiments of the invention, upregulating expression of the CAR or the tg-TCR is affected on day 12-14 from initiation of culture.

According to some embodiments of the invention, upregulating expression of the CAR or the tg-TCR is affected by mRNA electroporation.

According to some embodiments of the invention, the CAR or the tg-TCR is transiently expressed.

According to some embodiments of the invention, the CAR comprises at least one co-stimulatory domain.

According to some embodiments of the invention, the at least one co-stimulatory domain is selected from the group consisting of CD28, 2B4, CD137/4-1BB, CD134/OX40, Lsk, ICOS and DAP10.

According to some embodiments of the invention, the CAR comprises at least one activating domain.

According to some embodiments of the invention, the activating domain comprises a CD3ζ, FC-epsilon-R, or FcR-γ.

According to some embodiments of the invention, the CAR comprises at least one of a transmembrane domain and a hinge domain.

According to some embodiments of the invention, the transmembrane domain is selected from a CD8, a CD28 and a NKG2D.

According to some embodiments of the invention, the hinge domain is selected from a CD8 and a CD28.

According to some embodiments of the invention, the CAR comprises an antigen binding domain being an antibody or an antigen-binding fragment.

According to some embodiments of the invention, the antigen-binding fragment is a Fab or a scFv. In typical embodiments, the scFv is an anti-Her2 scFv. In some embodiments, the scFv is SEQ ID NO: 42. In some embodiments, the scFv is about 100% similar to, or about 95% similar to, or about 90% similar to, or about 85% similar to, or about 80% similar to SEQ ID NO: 42.

According to some embodiments of the invention, the disease is a malignant disease.

According to some embodiments of the invention, the malignant disease comprises HER2+ cells.

According to some embodiments of the invention, the malignant disease is a solid tumor or tumor metastasis.

According to some embodiments of the invention, the malignant disease is selected from the group consisting of a breast cancer, a gastric cancer, a gastroesophageal cancer, an oesophageal cancer, an ovarian cancer, an endometrial cancer, a lung cancer, an urothelial cancer and a bladder cancer.

According to some embodiments of the invention, the subject is a human subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Any aspect and/or embodiment described herein can be combined with any other aspect and/or embodiment described herein.

### BRIEF DESCRIPTION OF THE DRAWINGS

The above and further features will be more clearly appreciated from the following detailed description when taken in conjunction with the accompanying drawings.

FIG. 1 is a schematic representation of the anti-HER2 CAR genetic constructs.

FIGS. 2A, 2B, 2C and 2D are schematic illustrations showing different constructs engineered to express anti-HER2 CAR. FIG. 2A illustrates 501.A (501.1). FIG. 2B illustrates 501.B (501.2). FIG. 2C illustrates 501.C (501.3). FIG. 2D illustrates 501.D (501.4).

FIG. 3 is a schematic summary of the constructs demonstrating the appearance on the cells.

FIGS. 4A, 4B, 4C, 4D, 4E, 4F and 4G illustrate the sandwich flow cytometry method to determined CAR expression on the NK cells. (FIG. 4A) A schematic illustration of NK expressing anti-HER2 CAR and binding Her2 protein, detected by a specific anti-Her2 antibody. (FIGS. 4B-4G) Flow cytometry plots representing the specific determination of anti-HER2 CAR expressing on the electroporated cells only. Gate strategy for the staining was performed using a size gating on live cells (FIG. 4B) followed by staining via anti-Her2 antibody on control NK cells (FIG. 4C), electro (mock) control (FIG. 4D) and on NKs electroporated with CAR-B (FIG. 4E), CAR-C (FIG. 4F) and CAR-D (FIG. 4G), per FIG. 3.

FIG. 5 illustrates anti-HER2 CAR genetic constructs (FIG. 5A).

FIG. 6 illustrates HER2-CAR (501.1g-501.4g) expression on the NK cells 24 hours post-electroporation.

FIG. 7 is a flow cytometric analysis of co-culture of HER2-CAR NK and target cell mediated expression of CD107a, TNFalpha, IFNgamma, and GM-CSF.

FIG. 8 illustrates specific lysis percentages following a 6-hour HER2 CAR NK co-culture with SKOV3 cells (5:1 E:T).

FIG. 9 is a flow cytometric analysis of co-culture of HER2-CAR NK (501.1g-501.4g) and target cell mediated expression of CD107a, TNFalpha, IFNgamma, and GM-CSF.

FIG. 10 is a flow cytometric analysis of co-culture of HER2-CAR NK (501.1g-501.4g) and target cell mediated expression of MIP-1b.

FIG. 11 illustrates specific lysis percentages following a 6-hour HER2 CAR NK (501.1g-501.4g) co-culture with SKOV3 cells at 24-hours or 48-hours post-electroporation.

FIG. 12 illustrates CD107a, TNFalpha, IFNgamma expression in co-cultured HER2-CAR NK (501.3g or 501.4g) for 6 hours with SKOV3 (left column) or A549 cell lines (right column) at 24-, 48-, or 72-hours post-electroporation.

FIG. 13 illustrates GM-CSF, MIP1alpha, or MIP1beta expression in co-cultured HER2-CAR NK (501.3g or 501.4g) for 6 hours with SKOV3 (left column) or A549 cell lines (right column) at 24-, 48-, or 72-hours post-electroporation.

FIG. 14 illustrates specific lysis percentages following a 6 hour HER2 CAR NK (501.3g or 501.4g) co-culture with SKOV3 cells (5:1 or 1:1 E:T) at 24-, 48-, or 72-hours post-electroporation.

FIG. 15 illustrates specific lysis percentages following Herceptin addition to control (no CAR) NK cells, which are co-cultured with SKOV3 cells (5:1 or 1:1 E:T) at 24-, 48-, or 72-hours post-electroporation

FIG. 16 illustrates specific lysis percentages following a 6 hour HER2 CAR NK (501.3g or 501.4g) co-culture with A549 cells (5:1 or 1:1 E:T) at 24-, 48-, or 72-hours post-electroporation.

FIG. 17 illustrates specific lysis percentages following a 6 hour HER2 CAR NK (501.3g or 501.4g) co-culture with RPMI-8226 cells (5:1 or 1:1 E:T) at 24-, 48-hours post-electroporation.

FIG. 18 illustrates specific lysis percentages following a 6 hour HER2 CAR NK (501.1g-501.4g) co-culture with RPMI-8226 cells (5:1 or 1:1 E:T) at 24 hours post-electroporation.

FIG. 19 illustrates specific lysis percentages following a 3 hour HER2 CAR NK (501.3g or 501.4) co-culture with Raji cells (2:1 E:T) at 24 hours post-electroporation.

FIG. 20 illustrates specific lysis percentages following a 5 hour HER2 CAR NK (501.1g-501.4g) co-culture with allogeneic NK cells (5:1 E:T) at 24 hours post-electroporation.

FIG. 21 illustrates specific lysis percentages following a 5 hour HER2 CAR NK (501.3g or 501.4g) co-culture with allogeneic PBMCs (5:1 E:T) at 24 hours post-electroporation.

FIG. 22 illustrates flow cytometric analysis of TIGIT NK surface expression.

FIG. 23 illustrates flow cytometric analysis of surface CD62L, TRAIL, DNAM1, and LAG3.

### DETAILED DESCRIPTION

The present invention, in some embodiments thereof, relates to engineered Natural Killer (NK) cells and, more particularly, but not exclusively, to NK cells modified to express a chimeric antigen receptor (CAR) or a transgenic T cell receptor (tg-TCR) capable of binding HER2.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While reducing the present invention to practice, the present inventors have illustrated that NK cells can be tailored to target specific disease cells of interest while concomitantly having improved properties for an efficient immunotherapy.

As is shown hereinbelow and in the Examples section which follows, the present inventors have produced NK cells with improved properties by ex vivo expanding NK cell populations under culture conditions including nutrients, serum, IL-15 and nicotinamide (see general materials and experimental procedures section, below). In order to target the NK cells towards HER2 expressing cells, improve the anti-disease function and survival of the NK cells, expanded NK cells were modified to transiently express, by mRNA electroporation, anti-HER2 CAR (see Example 1).

Taken together, the ex vivo produced NK cells of the invention offer the solution of comprising high numbers, having both a high survival and a high functionality (e.g. high cytotoxicity) in vitro, and being engineered to target HER2 expressing cells of interest (e.g. cells of a solid tumor or metastasis). In typical embodiments, the ex vivo produced NK cells of the invention offer the solution of comprising high numbers, having both a high survival and a high functionality (e.g. high cytotoxicity) in vitro and in vivo, and being engineered to target HER2 expressing cells of interest (e.g. cells of a solid tumor or metastasis). In

### NK Cells of the Present Disclosure

The present disclosure provides a NK cell composition, wherein the NK cell is comprised of one or more chimeric antigen receptor (CAR) or a transgenic T cell receptor (tg-TCR). In typical embodiments, the CAR or tg-TCR is capable of binding HER2. In some embodiments, the NK cell comprises two chimeric antigen receptors (CAR) capable of binding HER2. In some embodiments, the NK cell comprises two chimeric antigen receptors (CAR) capable of binding HER2, wherein the CARs are not the same. In certain embodiments, the NK cell comprises two chimeric antigen receptors (CAR) capable of binding HER2, wherein the two CARs are not the same due to use of a different activation, co-stimulatory, transmembrane, hinge, or binding domain (e.g., scFv domains).

In some embodiments, the CAR comprises an antibody, antibody domain, or antibody fragment. In typical embodiments, the CAR comprises at least one scFv or Fab capable of binding HER2.

In typical embodiments, the CAR comprises at least one hinge domain. In some embodiments, the hinge domain is selected from CD28 or CD8.

In typical embodiments the CAR comprises at least one transmembrane domain. In some embodiments, the transmembrane domain is selected from CD28, CD8, or NK2D. In typical embodiments, the CAR comprises at least one hinge domain and at least one transmembrane domain.

In some embodiments, the CAR comprises a co-stimulatory domain. In certain embodiments, the co-stimulatory domain is selected from CD28, 4-1BB, 2B4, CD3zettaR, OX40, Lsk, ICOS, DAP10, and Fc fragment of IgE receptor Ig co-stimulatory domain. In certain embodiments, the CAR comprises at least one hinge domain, at least one transmembrane domain, and at least one co-stimulatory domain.

In some embodiments, the CAR comprises an activating domain. In certain embodiments, the activating domain is selected from CD3ζ, FcR-γ, and Fc-epsilon-R. In certain embodiments, the CAR comprises at least one hinge domain, at least one transmembrane domain, at least one co-stimulatory domain, and at least one activating domain.

In some embodiments, the cells in the population of nucleated cells further comprise a chemokine receptor or a mutant chemokine receptor. In some embodiments, the chemokine receptor is CXCR4 or mutant CXCR4. In some embodiments, the mutant CXCR4 is a CXCR4^{R334X} mutant. In some embodiments, the mutant CXCR4 is SEQ ID NO: 40.

In certain embodiments, the CAR further comprises a signal peptide or leader peptide.

### NK Cell Fractions of the Present Disclosure

The present disclosure provides compositions comprising an NK cell fraction comprising a population of nucleated cells.

In some aspects, the population of nucleated cells can comprise at least about 1.0×10⁶, or at least about 5.0×10⁶, or at least about 1.0×10⁷, or at least about 5.0×10⁷, or at least about 1.0×10⁸, or at least about 5.0×10⁸, or at least about 1.0×10⁹, or at least about 5.0×10⁹, or at least about 1.0×10¹⁰, or at least about 5.0×10¹⁰, or at least about 1.0×10¹¹, or at least about 5.0×10¹¹, or at least about 1.0×10¹², or at least about 5.0×10¹² nucleated cells. In some aspects, the population of nucleated cells can comprise at least about at least about 1.0×10⁶ cells. In some aspects, the population of nucleated cells can comprise at least about at least about 17.5×10⁸ cells. In some aspects, the population of nucleated cells can comprise at least about at least about 35×10⁸. In some aspects, the population of nucleated cells can comprise at least about at least about 2.5×10⁹ cells. In some aspects, the population of nucleated cells can comprise at least about at least about 5×10⁹ cells.

In some aspects, at least about 60%, or at least about 65%, or at least about 70%, or at least about 75%, or at least about 80%, or at least about 85%, or at least about 90%, or at least about 95%, or at least about 99% of the cells in the population of nucleated cells are viable. In some aspects, at least about 70% of the cells in the population of nucleated cells are viable.

In some aspects, at least about at least about 60%, or at least about 65%, or at least about 70%, or at least about 75%, or at least about 80%, or at least about 85%, or at least about 90%, or at least about 95%, or at least about 99% of the cells in the population of nucleated cells are CD56+. In some aspects, at least about 70% of cells in the population of nucleated cells are CD56+.

In some aspects, about 80% to about 99%, or about 85% to about 95%, or about 90 to about 95% of the cells in the population of nucleated cells are CD56+. In some aspects, about 90 to about 95% of the cells in the population of nucleated cells are CD56+.

In some aspects, no more than about 0.1%, or no more than about 0.2%, or no more than about 0.3%, or no more than about 0.4%, or no more than about 0.5%, or no more than about 0.6%, or no more than about 0.7%, or no more than about 0.8%, or no more than about 0.9%, or no more than about 1.0% of cells in the population of nucleated cells is CD3+. In some aspects, no more than 0.5% of cells in the population of nucleated cells are CD3+.

In some aspects, about 0.1% to about 0.5%, or about 0.2% to about 0.3% of cells in the population of nucleated cells are CD3+. In some aspects, about 0.2% to about 0.3% of cells in the population of nucleated cells are CD3+.

In some aspects, at least about at least about 60%, or at least about 65%, or at least about 70%, or at least about 75%, or at least about 80%, or at least about 85%, or at least about 90%, or at least about 95%, or at least about 99% of the cells in the population of nucleated cells are CD56+/CD3−. In some aspects, at least about 70% of cells in the population of nucleated cells are CD56+/CD3−. In some aspects, at least about 99% of the cells in the population of nucleated cells are CD56+/CD3−.

In some aspects, about 80% to about 99%, or about 85% to about 95%, or about 90 to about 95% of the cells in the population of nucleated cells is CD56+/CD3−. In some aspects, about 90 to about 95% of the cells in the population of nucleated cells is CD56+/CD3−.

In some aspects, no more than about 0.1%, or no more than about 0.2%, or no more than about 0.3%, or no more than about 0.4%, or no more than about 0.5%, or no more than about 0.6%, or no more than about 0.7%, or no more than about 0.8%, or no more than about 0.9%, or no more than about 1.0% of cells in the population of nucleated cells is CD56−/CD3+. In some aspects, no more than 0.5% of cells in the population of nucleated cells are CD56−/CD3+.

In some aspects, about 0.1% to about 0.5%, or about 0.2% to about 0.3% of cells in the population of nucleated cells are CD56−/CD3+. In some aspects, about 0.2% to about 0.3% of cells in the population of nucleated cells are CD56−/CD3+.

In some aspects, no more than about 5%, or no more than about 10%, or no more than about 15%, or no more than about 20%, or no more than about 25% of cells in the population of nucleated cells are CD19+. In some aspects, no more than about 10% of cells in the population of nucleated cells are CD19+.

In some aspects, no more than about 5%, or no more than about 10%, or no more than about 15%, or no more than about 20%, or no more than about 25% of cells in the population of nucleated cells are CD14+. In some aspects, no more than about 10% of cells in the population of nucleated cells are CD14+.

In some aspects, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75% of cells in the population of nucleated cells are CD62L+, or at least 80% of cells in the population of nucleated cells are CD62L+. In some aspects, at least about 60% of the cells in the population of nucleated cells are CD62L+.

In some aspects, no more than about 10%, or no more than about 20%, or no more than about 30%, or no more than about 40%, or no more than about 50%, or no more than about 60%, or no more than about 65%, or no more than about 70%, or no more than about 75% of cells in the population of nucleated cells are LAG3+. In some aspects, no more than about 70% of cells in the population of nucleated cells are LAG3+.

In some aspects, at least about 10%, or at least about 20%, or at least about 30%, or at least about 40%, or at least about 50%, or at least about 55%, or at least about 60%, or at least about 70%, or at least about 75% of cells in the population of nucleated cells are TRAIL+. In some aspects, at least about 60% of cells in the population of nucleated cells are TRAIL+.

In some aspects, at least about 10%, or at least about 20%, or at least about 30%, or at least about 40%, or at least about 50%, or at least about 55%, or at least about 60%, or at least about 70%, or at least about 75% of cells in the population of nucleated cells are DNAM1+. In some aspects, at least about 60% of cells in the population of nucleated cells are DNAM1+.

Any of the aforementioned phenotypic parameters can be combined with any of the other aforementioned phenotypic parameters.

Accordingly, in a non-limiting example, the present disclosure provides NK cell fraction comprising a population of nucleated cells, wherein the population comprises at least 1.0×10⁶ nucleated cells, wherein at least about 70% of the cells in the population are viable, wherein:
at least about 70% of cells in the population are CD56+;
no more than about 0.5% of the cells in the population are CD3+;
no more than about 10% of the cells in the population are CD19+;
no more than about 10% of the cells in the population are CD14+;
at least about 60% of the cells in the population are CD62L+;
no more than about 70% of the cells in the population are LAG3+;
at least about 60% of the cells in the population are TRAIL+;
at least about 60% of the cells in the population are DNAM1+.

The present disclosure also provides a cryopreserved NK cell fraction, comprising any of the NK cell fractions described herein and DMSO. In some aspects, the concentration of DMSO can be about 1% v/v, or about 2% v/v, or about 3% v/v, or about 4% v/v, or about 5% v/v, or about 6% v/v, or about 7% v/v, or about 8% v/v, or about 9% v/v, or about 10% v/v, or about 11% v/v, or about 12% v/v, or about 13% v/v, or about 14% v/v, or about 15% v/v. In some aspects, the concentration of DMSO can be about 10% v/v.

In some aspects, a cryopreserved NK cell fraction can be stable for at least about 1 month, or at least about 2 months, or at least about 3 months, or at least about 4 months, or at least about 5 months, or at least about 6 months, or at least about 7 months, or at least about 9 months, or at least about 10 months, or at least about 12 months. In some aspects, a cryopreserved NK cell fraction can be stable at about −80° C. for at least about 1 month, or at least about 2 months, or at least about 3 months, or at least about 4 months, or at least about 5 months, or at least about 6 months, or at least about 7 months, or at least about 9 months, or at least about 10 months, or least about 12 months.

### Potency Assays of the Present Disclosure

The present disclosure provides a first potency assay, the assay comprising the steps of:
a) incubating an NK CAR cell fraction of the present disclosure and a plurality of target cells, wherein the plurality of target cells is stained with at least one proliferation stain;
b) determining the cell death percentage in the plurality of target cells.

In some aspects of the first potency assay, the incubation conditions of step (a) can further comprise at least one anti-cancer therapeutic monoclonal antibody.

In some aspects of the first potency assay, the target cells can be SKOV3 cells.

In some aspects of the first potency assay, the target cells can be A549 cells. In some aspects of the first potency assay, the target cells can be RPMI-8226, CAG and U266, Raji, or K562.

In some aspects of the first potency assay, the target cells can be SKOV3 cells or A549 cells, and the incubation conditions of step (a) can further comprise Herceptin. In some aspects, the Herceptin can be present at a concentration of about 1 μg/ml or 10 μg/ml.

As would be appreciated by the skilled artisan, determining the cell death percentage in the plurality of target cells in step (b) of the first potency assay can be accomplished using any standard technique known in the art for determining cell death percentages. In a non-limiting example, determining the cell death percentage in the plurality of target cells can comprise: i) staining the NK cell fraction and plurality of target cells incubated in step (a) with at least one viability stain; ii) using fluorescent activated cell sorting (FACS) to separate the plurality of target cells from the NK cell fraction; and iii) using the viability stain to determine the cell death percentage in the plurality of target cells sorted in separated in step (ii).

In some aspects, the at least one proliferation stain can be carboxyfluorescein diacetate, succinimidyl ester (CFSE). As would be appreciated by the skilled artisan, any proliferation stain known in the art can be used in the first potency assay, described herein.

In some aspects, the at least one viability stain can be Helix NP™ Blue (also known as Sytox™ Blue). As would be appreciated by the skilled artisan, any proliferation stain known in the art can be used in the first potency assay.

In some aspects, the incubation in step (a) of the first potency assay can be performed at about 37° C.

In some aspects, the incubation in step (a) of the first potency assay can be performed for at least about three hours.

In some aspects, the ratio of the number of cells in the NK cell fraction to the number of cells in the plurality of target cells in step (a) of the first potency assay can be about 2.5:1, or about 3:1 or about 5:1, or about 10:1.

In some aspects, an NK cell fraction of the present disclosure can be characterized in that when the NK cell fraction is tested using first potency assay described above, wherein target cells are SVO3 cells, the cell death percentage in the target cells is at least 40% at a 5:1 E:T ratio. In some aspects, an NK cell fraction of the present disclosure can be characterized in that when the NK cell fraction is tested using first potency assay described above, wherein target cells are SVO3 cells, the cell death percentage in the target cells is at least 50% at a 5:1 E:T ratio. In some aspects, an NK cell fraction of the present disclosure can be characterized in that when the NK cell fraction is tested using first potency assay described above, wherein target cells are SVO3 cells, the cell death percentage in the target cells is at least 35% at a 1:1 E:T ratio.

In some aspects, an NK cell fraction of the present disclosure can be characterized in that when the NK cell fraction is tested using first potency assay described above, wherein target cells are SKOV3 or A549 cells, the cell death percentage in the target cells is at least 50%, or at least 60%, or at least 70%, or at least 80% at a 1:1 or 5:1 E:T ratio.

The present disclosure provides a second potency assay, the assay comprising the steps of:
a) incubating an NK cell fraction of the present disclosure and a plurality of target cells, wherein the NK cell fraction is stained with at least one anti-CD107α antibody comprising a detectable label;
b) treating the NK cell fraction and the plurality of target cells incubated in step (a) with one or more protein trafficking inhibitors and further incubating the NK cell fraction and the plurality of target cells;
c) staining the NK cell fraction and plurality of target cells with:
   at least one viability stain;
   at least one anti-CD56 antibody comprising a detectable label
d) fixing the NK cell fraction and the plurality of target cells;
e) permeabilizing the NK cell fraction and the plurality of target cells;
f) staining the NK cell fraction and plurality of target cells with any one of:
   i) an anti-IFNγ antibody comprising a detectable label;
   ii) an anti-TNFα antibody comprising a detectable label;
   iii) an anti-GM-CSF antibody comprising a detectable label;
   iv) an anti-MIP1alpha antibody comprising a detectable label;
   v) an anti-MIP1beta antibody comprising a detectable label;
   vi) an anti-CD62L antibody comprising a detectable label;
   vii) an anti-TRAIL antibody comprising a detectable label;
   viii) vii) an anti-DNAM1 antibody comprising a detectable label;
   IX) vii) an anti-DNAM1 antibody comprising a detectable label;
g) determining at least one of:
   g₁) the percentage of viable cells stained with the at least one anti-CD56 antibody that are also stained with the at least one anti-CD107α antibody (i.e. number of CD107a+/CD56+ cells÷number of CD56+ cells×100%);
   g₂) the percentage of viable cells stained with the at least one anti-CD56 antibody that are also stained with the at least one anti-IFNγ antibody (i.e. number of IFNγ+/CD56+ cells÷number of CD56+ cells×100%); and
   g₃) the percentage of viable cells stained with the at least one anti-CD56 antibody that are also stained with the at least one anti-TNFα antibody (i.e. number of TNFα+/CD56+ cells÷number of CD56+ cells×100%).

In some aspects of the second potency assay, the target cells can be SKOV3 cells.

In some aspects of the second potency assay, the target cells can be A549 cells.

In some aspects, the at least one viability stain can be Zombie Violet™ Viability Dye. As would be appreciated by the skilled artisan, any proliferation stain known in the art can be used in the first potency assay.

In some aspects, the one or more protein trafficking inhibitors can comprise brefeldin, GolgiStop™ Protein Transport Inhibitor (BD), a combination of brefeldin and GolgiStop™ Protein Transport Inhibitor, or any other protein tracking inhibitors known in the art.

In some aspects of the second potency assay, the further incubation in step (b) is performed at about 37° C.

In some aspects of the second potency assay, the further incubation in step (b) is performed for at least about 37° C.

As would be appreciated by the skilled artisan, determining at least one (g₁)-(g₃) of step (g) can be accomplished using any standard technique known in the art for determining percentages of cells labeled with antibodies comprising detectable labels, including, but not limited to fluorescent activated cell sorting (FACS).

In some aspects of the second potency assay, step (g) can comprise determining each of (g₁)-(g₃).

In some aspects, an NK cell fraction of the present disclosure can be characterized in that when the NK cell fraction is tested using second potency assay described above, wherein target cells are SKOV3 cells, the percentage of viable cells stained with the at least one anti-CD56 antibody that are also stained with the at least one anti-CD107α antibody is at least 60% (1:3 ratio of E:T).

According to one aspect of the present invention there is provided a method of ex vivo producing natural killer (NK) cells expressing a chimeric antigen receptor (CAR) or a transgenic T cell receptor (tg-TCR) capable of binding HER2, the method comprising:
(a) expanding a population of NK cells by a method comprising:
   (i) culturing a population of NK cells under conditions allowing for cell proliferation, wherein the conditions comprise providing an effective amount of nutrients, serum, IL-15 and nicotinamide; and
   (ii) supplementing the population of NK cells with an effective amount of fresh nutrients, serum, IL-15 and nicotinamide 5-10 days following step (i) to produce expanded NK cells;
   so as to obtain an ex vivo expanded population of NK cells, and
(b) upregulating expression of a CAR or a tg-TCR capable of binding HER2 in the ex vivo expanded population of NK cells, thereby producing the NK cells expressing the CAR or the tg-TCR capable of binding the HER2.

As used herein, the term “natural killer cells” or “NK cells” refers to large granular lymphocytes involved in the innate immune response. Functionally, NK cells exhibit cytolytic activity against a variety of targets via exocytosis of cytoplasmic granules containing a variety of proteins, including perforin, granulysin and granzyme proteases. Killing is triggered in a contact-dependent, non-phagocytotic process which does not require prior sensitization to an antigen.

Human NK cells are characterized by the presence of the cell-surface markers CD16 and CD56, and the absence of the T cell receptor (CD3). Human bone marrow-derived NK cells are further characterized by the CD2⁺CD16⁺CD56⁺CD3⁻ phenotype, further typically containing the T-cell receptor zeta-chain [zeta-TCR], and often characterized by the presence of NKp46, NKp30 or NKp44. Non-NK cells such as NKT cells or CD8NKT possess characteristics and cell-surface markers of both T cells and NK cells (e.g. expression of CD3).

In one embodiment, the population of NK cells comprise mature NK cells. As used herein, the term “mature NK cell” is defined as a committed NK cell, having characteristic surface markers and NK cell function, and lacking the potential for further differentiation. As use herein, mature NK cells include, but are not limited to CD₅₆^{bright} cells, which can proliferate and produce abundant cytokines; CD56^{dim} cells, exhibiting robust cytotoxicity; CD56^{bright}CD94^{high} and CD56^{dim}CD94^{high} cells. Cell surface expression of the CD56, CD3, CD94 and other markers can be determined, for example, by FACS analysis or immunohistological staining techniques.

In another embodiment, the population of NK cells comprise NK progenitor cells, or mixed populations of NK progenitor cells and mature NK cells. As used herein, the term “progenitor” refers to an immature cell capable of dividing and/or undergoing differentiation into one or more mature effector cells. Lymphocyte progenitors include, for example, pluripotent hematopoietic stem cells capable of giving rise to mature cells of the B cell, T cell and NK lineages. In the B cell lineage (that is, in the developmental pathway that gives rise to mature B cells), progenitor cells also include pro-B cells and pre-B cells characterized by immunoglobulin gene rearrangement and expression. In the T and NK cell lineages, progenitor cells also include bone-marrow derived bipotential T/NK cell progenitors [e.g., CD34(+)CD45RA(hi)CD7(+) and CD34(+)CD45RA(hi)Lin(−)CD10(+) cells], as well as intrathymic progenitor cells, including double negative (with respect to CD4 and CD8) and double positive thymocytes (T cell lineage) and committed NK cell progenitors.

The NK cells of some embodiments of the invention are isolated cells.

The term “isolated” refers to at least partially separated from the natural environment e.g., from a tissue, e.g., from a human body.

The term “population of NK cells” refers to a heterogeneous mixture of NK cells, such as at different stages of maturity, having different signatures, or having different functions.

NK cells of some embodiments of the present invention may be derived from any source which comprises such cells. NK cells are found in many tissues, and can be obtained, for example, from lymph nodes, spleen, liver, lungs, intestines, deciduous and can also be obtained from induced pluripotent stem cells (iPSCs) or embryonic stem cells (ESC). Typically, cord blood, peripheral blood, mobilized peripheral blood and bone marrow (e.g. CD34+ cells), which contain heterogeneous lymphocyte cell populations, are used to provide large numbers of NK cells for research and clinical use.

According to one embodiment, NK cells are obtained from peripheral blood.

Any blood collection method may be employed according to the present teachings. For example, a common method for collecting blood fractions is apheresis, in which whole donor blood is separated into blood components (e.g. plasma, leukocytes and erythrocytes), typically by centrifugation, selected components are drawn off for manipulation (e.g. culturing of leukocyte fractions) and the remainder is returned to the donor. Many suitable apheresis devices are commercially available. Typically, apheresis applies to separation of blood components from the peripheral blood of the donor.

Lymphocyte fractions, such as “buffy coat” or apheresis units can be processed to enrich or purify or isolate specific defined populations of cells. The terms “purify” and “isolate” do not require absolute purity; rather, these are intended as relative terms. Thus, for example, a purified lymphocyte population is one in which the specified cells are more enriched than such cells are in its source tissue. A preparation of substantially pure lymphocytes can be enriched such that the desired cells (e.g. NK cells) represent at least 10%, 20%, 30%, 40%, 50% or more of the total cells present in the preparation. Methods for enriching, purifying and isolating lymphocytes are well known in the art, and appropriate methods can be selected based on the desired population. For example, lymphocyte enrichment can be performed using commercially available preparations for negatively selecting unwanted cells, such as FICOLL-HYPAQUE™ and other density gradient mediums formulated for the enrichment of whole lymphocytes, T cells or NK cells.

Methods of selection of NK cells from blood, bone marrow, lymphocyte preparations (e.g. apheresis units) or tissue samples are well known in the art (see, for example, U.S. Pat. No. 5,770,387 to Litwin et al., which is incorporated herein in its entirety by reference). Most commonly used are protocols based on isolation and purification of CD56+ cells, usually following mononuclear cell fractionation, and depletion of non-NK cells such as CD3+, CD19+, CD14+, CD34+ and/or CD133+ cells and the like. Combinations of two or more protocols can be employed to provide NK cell populations having greater purity from non-NK contaminants. The purity of the NK cell preparation is of great significance for clinical applications, as non-NK cells, such as T-cells and NKT cells, contribute to antigen-specific reactions such as graft versus host disease (GVHD), compromising the potential benefits of NK cell transplantation. Commercially available kits for isolation of NK cells include one-step procedures (for example, CD56 microbeads and CD56+, CD56+CD16+ isolation kits from Miltenyi Biotec, Auburn CA), and multistep procedures, including depletion, or partial depletion, of CD3+ or depletion with non-NK cell antibodies recognizing and removing T cells (for example, OKT-3), B cells, stem cells, dendritic cells, monocytes, granulocytes and erythroid cells.

Methods for selection of NK cells according to phenotype include, but are not limited to, immunodetection and FACS analysis. In specific embodiments, the NK cell population is depleted of CD3+ cells, CD14+ cells, CD19+ cells, etc. or is selected for CD56+ cells by immunomagnetic selection, for example, using a CliniMACS (LS Column, Miltenyi Biotec).

Thus, in certain embodiments, the NK cell population is selected or enriched for NK cells, and can be a CD3-depleted NK cell fraction.

According to another embodiment, the NK cell population is selected or enriched for NK cells, and can be a CD56+ NK cell fraction.

According to one embodiment, the NK cell population comprises CD56+CD16+CD3− cells and/or CD56+CD16−CD3− cells.

In specific embodiments, the population of cells comprising NK cells at the initiation of culture (i.e. before ex vivo expansion) comprise at least 10%, at least 20%, at least 30%, at least 40%, at least 50%, at least 60%, at least 70%, at least 80%, at least 90% or more CD3−/CD56+ cells.

In specific embodiments, the population of cells comprising NK cells at the initiation of culture comprise at least 40%, at least 50%, at least 60%, at least 70%, at least 80%, at least 90% or more CD3−/CD56+ cells.

In some embodiments, the population of cells comprising NK cells at the initiation of culture comprise between 10%-30% CD3−/CD56+ cells, 10%-50% CD3−/CD56+ cells, 20%-40% CD3−/CD56+ cells, 20%-60% CD3−/CD56+ cells, 30%-50% CD3−/CD56+ cells, 30%-70% CD3−/CD56+ cells, 40%-60% CD3−/CD56+ cells, 40%-80% CD3−/CD56+ cells, 50%-70% CD3−/CD56+ cells, 50%-90% CD3−/CD56+ cells, 60%-80% CD3−/CD56+ cells, 60%-100% CD3−/CD56+ cells, 70%-90% CD3−/CD56+ cells, or 80%-100% CD3−/CD56+ cells.

It will be appreciated that at the initiation of culture the population of cells comprising NK cells may comprise residual monocytes, B cells, T cells, dendritic cells and the like, however, these are ablated through the course of ex vivo culture.

In some embodiments, the NK cell population is devoid of erythrocytes. Thus, in some embodiments, prior to or following CD3+/CD14+/CD19+ cell depletion or CD56+ cell selection, the NK cell fraction undergoes red blood cell (RBC) lysis before culturing. In specific embodiments, red blood cell lysis is accomplished using ammonium chloride potassium (ACK) buffer (Gibco, Thermo Fischer Scientific).

According to some embodiments, NK cells can be cultured from fresh cell populations, while other embodiments culture NK cells from stored cell populations (such as cryopreserved and thawed cells) or previously cultured cell populations.

According to one embodiment, the method comprises expanding the population of NK cells.

The term “expanded” when relating to a population of NK cells refers to increased numbers of NK cells through ex vivo or in vitro expansion (proliferation) without negatively affecting the viability or functionality of the cells.

According to one embodiment, fold expansion of the NK cells of some embodiments of the invention is between 2 to 12, e.g. between 3 to 11, e.g. between 4 to 10 (i.e. from day 0 to day 14-16 of culture).

Expansion of NK cells is typically affected in an ex vivo cell culture.

Previous studies have demonstrated that NK cells cultured with growth factors and nicotinamide and/or other nicotinamide moiety, for as little as 7 days, or as many as 3 weeks resulted in enhanced, preferential proliferation and/or functionality as compared to cells cultured with cytokines but with less than 0.1 mM nicotinamide and/or other nicotinamide moiety (see PCT Publication WO2011/080740). In preparing a clinically suitable NK cells for immunotherapy, it is desirable to provide significant ex vivo NK cell expansion while retaining therapeutically advantageous functionality of the expanded NK cells, without requiring lengthy culture duration.

According to one embodiment, expansion of NK cells is affected for a period of 7-30 days, 7-25 days, 7-21 days, 7-14 days, 10-24 days, 10-21 days, 10-18 days, 10-15 days, 10-12 days, 12-21 days, 12-18 days, 12-15 days, 14-21 days, 14-18 days, 14-16 days, 14-15 days, 16-21 days, 16-18 days, or 18-21 days. In particular embodiments, expansion of NK cells is affected for a period of 12-16 days. In particular embodiments, expansion of NK cells is affected for a period of 12-14 days.

According to a specific embodiment, expansion of NK cells is affected for a period of 12-18 days.

According to a specific embodiment, expansion of NK cells is affected for a period of 14-16 days.

Ex vivo culturing of NK cells can be effected, according to this aspect of the present invention, by providing NK cells ex vivo with conditions for cell proliferation and ex vivo culturing the NK cells with a nicotinamide moiety, thereby ex vivo expanding the population of NK cells.

As used herein “culturing” includes providing the chemical and physical conditions (e.g., temperature, gas) which are required for NK cell maintenance, as well as nutrients and growth factors. In one embodiment, culturing the NK cells includes providing the NK cells with conditions for NK cell proliferation. Examples of chemical conditions which may support NK cell proliferation include but are not limited to buffers, nutrients, serum, vitamins and antibiotics as well as cytokines and other growth factors which are typically provided in the growth (i.e., culture) medium. In a particular embodiment, conditions for cell proliferation comprise nutrients, serum and cytokine(s). According to a specific embodiment, the growth factors comprise, for example, IL-15, IL-2, IL-7, IL-12, IL-21, SCF and FLT3.

According to one embodiment, conditions allowing for cell proliferation enable the NK cells to double every 1 day, 1.25 day, 1.5 day, 1.75 day, or 2.0 days.

In one embodiment, the NK culture medium includes a minimal essential medium (MEM), such as MEMα (BI, Bet HaEmek, Israel) and serum. In some embodiments, the serum is provided at 2-20%, 5-15% or 5-10% of the culture medium. In specific embodiments, the serum is human serum, provided at 10% of the culture medium. In a particular embodiment, the culture medium is MEMα comprising 10% Human AB Serum (Sigma-Aldrich, St. Louis, MO). Other media suitable for use with the invention include, but are not limited to Glascow's medium (Gibco Carlsbad CA), RPMI medium (Sigma-Aldrich, St Louis MO) or DMEM (Sigma-Aldrich, St Louis MO). It will be noted that many of the culture media contain nicotinamide as a vitamin supplement for example, MEMα (8.19 mM nicotinamide), RPMI (8.19 pM nicotinamide), DMEM (32.78 pM nicotinamide) and Glascow's medium (16.39 pM nicotinamide), however, the methods of the present invention relate to exogenously added nicotinamide supplementing any nicotinamide and/or nicotinamide moiety included the medium's formula, or that resulting from overall adjustment of medium component concentrations.

According to one embodiment, culturing the NK cells under conditions allowing for cell proliferation comprises providing the cells with nutrients, serum and cytokines. In some embodiments the at least one growth factor includes cytokines and/or chemokines (e.g. IL-15, IL-2, IL-7, IL-12, IL-21, SCF and FLT3). Cytokines and other growth factors are typically provided in concentrations ranging from 0.5-100 ng/ml, or 1.0-80 ng/ml, more typically 5-750 ng/ml, yet more typically 5.0-50 ng/ml (up to 10× such concentrations may be contemplated), and are available commercially, for example, from Perpo Tech, Inc., Rocky Hill, NJ, USA. In one embodiment, conditions allowing for cell proliferation includes providing the cytokine interleukin 15 (IL-15). In specific embodiments, the population of NK cells are cultured with 20 ng/ml IL-15.

Further, it will be appreciated in this respect that novel cytokines are continuously discovered, some of which may find uses in the methods of NK cell proliferation of the present invention.

The culture medium typically also comprises antibiotics, such as but not limited to, gentamicin, penicillin or streptomycin.

For applications, in which cells are introduced (or reintroduced) into a human subject, it is often preferable to use serum-free formulations, such as AIM v® serum free medium for lymphocyte culture or MARROWMAX® bone marrow medium. Such medium formulations and supplements are available from commercial sources such as Invitrogen (GIBCO) (Carlsbad, CA, USA). The cultures can be supplemented with amino acids, antibiotics, and/or with cytokines to promote optimal viability, proliferation, functionality and/or and survival.

According to one embodiment, the population of NK cells is cultured with nutrients, serum, a cytokine (e.g. IL-15) and nicotinamide and/or a nicotinamide moiety. As used herein, the term “nicotinamide moiety” refers to nicotinamide as well as to products that are derived from nicotinamide, derivatives, analogs and metabolites thereof, such as, for example, NAD, NADH and NADPH, which are capable of effectively and preferentially enhancing NK cell proliferation and/or activation. Nicotinamide derivatives, analogs and metabolites can be screened and evaluated for their effect on ex vivo NK proliferation in culture by addition to NK cultures maintained as described herein below, addition to functional assays such as killing and motility assays, or in automated screening protocols designed for high-throughput assays well known in the art, and further discussed below.

As used herein, the phrase “nicotinamide analog” refers to any molecule that is known to act similarly to nicotinamide in the abovementioned or similar assays. Representative examples of nicotinamide analogs can include, without limitation, benzamide, nicotinethioamide (the thiol analog of nicotinamide), nicotinic acid and a-amino-3-indolepropionic acid.

The phrase “nicotinamide derivative” further refers to any structural derivative of nicotinamide itself or of an analog of nicotinamide. Examples of such derivatives include, without limitation, substituted benzamides, substituted nicotinamides and nicotinethioamides and N-substituted nicotinamides and nicotinthioamides, 3-acetylpiridine and sodium nicotinate. In one particular embodiment of the invention the nicotinamide moiety is nicotinamide.

Nicotinamide or nicotinamide moiety concentrations suitable for use in some embodiments of the present invention are typically in the range of about 0.5 mM to about 50 mM, about 1.0 mM to about 25 mM, about 1.0 mM to about 15 mM, about 1.0 mM to about 10 mM, about 2.5 mM to about 20 mM, about 2.5 mM to about 10 mM, about 5.0 mM to about 10 mM. Exemplary effective concentrations of nicotinamide can be of about 0.5 mM to about 15 mM, 1.0 mM to about 10.0 mM, typically 2.5, 5.0 or 7.0 mM, based on the effect of these concentrations of nicotinamide on proliferation and NK cell function.

According to specific embodiments of the invention, nicotinamide is provided at a concentration (mM) of about 0.5, about 0.75, about 1.0, about 1.25, about 1.5, about 1.75, about 2.0, about 2.25, about 2.5, about 2.75, about 3.0, about 3.25, about 3.5, about 3.75, about 4.0, about 4.25, about 4.5, about 4.75, about 5.0, about 5.25, about 5.5, about 5.75, about 6.0, about 6.25, about 6.5, about 6.75, about 7.0, about 7.25, about 7.5, about 7.75, about 8.0, about 8.25, about 8.5, about 8.75, about 9.0, about 9.25, about 9.5, about 9.75, about 10.0, about 11.0, about 12.0, about 13.0, about 14.0, about 15.0, about 16.0, about 17.0, about 18.0 or about 20.0 mM. All effective intermediate concentrations are contemplated. In specific embodiments, conditions allowing proliferation comprise between 1.0 to 10.0 mM nicotinamide. In specific embodiments, conditions allowing proliferation comprise 5.0 mM nicotinamide. In other specific embodiments, conditions allowing proliferation comprise 7.0 mM nicotinamide.

Suitable concentrations of the nicotinamide and/or nicotinamide moiety can be determined according to any assay of NK proliferation and/or activity, for example, cell culture or function. Suitable concentration of nicotinamide is a concentration which use thereof in culture “enhances”, or results in a net increase of proliferation and/or function of NK cells in culture, compared to “control” cultures having less than 0.1 mM of the nicotinamide and tested from the same NK cell source (e.g. cord blood, bone marrow or peripheral blood preparation), in the same assay and under similar culture conditions (duration of exposure to nicotinamide, time of exposure to nicotinamide).

In some studies, ex vivo expansion of purified NK cells by culture with nutrients, serum, cytokines and nicotinamide does not require replenishing the medium or manipulation over the culture period, while other studies have advocated culture medium replenishment (“refeeding”) at different intervals during the NK cell culture. In certain embodiments of the present invention, the population of NK cells is “re-fed” during the culture period. Thus, in specific embodiments, expanding NK cells comprises supplementing the population of NK cells with fresh nutrients, serum, IL-15 and nicotinamide 8-10 days following initiation of the ex vivo culture. In some embodiments, supplementing is provided between 4-12 days following initiation of the ex vivo culture, between 5-10 days following initiation of the ex vivo culture, or between 6-9 days following initiation of culturing of the NK cells.

In some embodiments, supplementing (or “refeeding”) the NK cells in a culture does not comprise removing medium from the NK cell culture. In some embodiments, supplementing (or “refeeding”) comprises removing about 30-80%, about 40-70% or about 45-55% of the medium of the NK cell culture, and replacing that with a similar (e.g. equivalent) volume of fresh medium having the same composition and level of nutrients, serum, cytokines (e.g. IL-15) and nicotinamide as the removed medium. In other embodiments, culture volume following refeeding reaches approximately twice the original culture volume at initiation of the NK cell culture (“seeding”).

NK cell populations can be cultured using a variety of methods and devices. Selection of culture apparatus is usually based on the scale and purpose of the culture. Scaling up of cell culture preferably involves the use of dedicated devices. Apparatus for large scale, clinical grade NK cell production is detailed, for example, in Spanholtz et al. (PLoS ONE (2010) 5:e9221) and Sutlu et al. (Cytotherapy (2010), Early Online 1-12). In some embodiments, culturing the NK cells is effected in flasks, at a cell density of 100-4000×10⁶ cells per flask. In specific embodiments, culturing the NK cells (e.g. initiation of the ex vivo culture and/or “re-feeding”) is effected in flasks, at a cell density of 200-300×10⁶ cells per flask. In certain embodiments, the flasks are flasks comprising a gas-permeable membrane, such as the G-Rex culture device (G-Rex 100M or closed system G-Rex MCS, WolfWilson, St Paul MN).

Seeding the population of NK cells in culture flasks, such as the G-Rex culture device, can be affected at various densities depending on the size and volume of the culture device. A person of skill in the art is capable of making such a determination. According to one embodiment, the population of NK cells are seeded at a density of 0.01×10⁶ cells/ml to 10×10⁶ cells/ml, 0.01×10⁶ cells/ml to 7.5×10⁶ cells/ml, 0.01×10⁶ cells/ml to 5×10⁶ cells/ml, 0.1×10⁶ cells/ml to 10×10⁶ cells/ml, 0.1×10⁶ cells/ml to 7.5×10⁶ cells/ml, 0.1×10⁶ cells/ml to 5×10⁶ cells/ml, 0.1×10⁶ cells/ml to 2.5×10⁶ cells/ml, 0.1×10⁶ cells/ml to 1×10⁶ cells/ml, 0.25×10⁶ cells/ml to 10×10⁶ cells/ml, 0.25×10⁶ cells/ml to 7.5×10⁶ cells/ml, 0.25×10⁶ cells/ml to 5×10⁶ cells/ml, 0.25×10⁶ cells/ml to 2.5×10⁶ cells/ml, or 0.25×10⁶ cells/ml to 1×10⁶ cells/ml. According to a specific embodiment, the population of NK cells are seeded at a density of 0.25×10⁶ cells/ml to 0.5×10⁶ cells/ml, e.g. 0.35×10⁶ cells/ml to 0.4×10⁶ cells/ml.

It will be appreciated that the density of cells in the culture flask increases with proliferation of the cells over the duration of the culture. Thus, in some embodiments, over the course of expansion in culture, the NK cells of the population of NK cells are cultured at a cell density of 10-4000×10⁶ cells per flask, 25-4000×10⁶ cells per flask, 50-4000×10⁶ cells per flask, 100-4000×10⁶ cells per flask, 20-3000×10⁶ cells per flask, 100-3000×10⁶ cells per flask, 200-3000×10⁶ cells per flask, 30-2000×10⁶ cells per flask, 100-2000×10⁶ cells per flask, 300-2000×10⁶ cells per flask, 40-1000×10⁶ cells per flask, 100-1000×10⁶ cells per flask, 400-1000×10⁶ cells per flask, 100-800×10⁶ cells per flask, 250-800×10⁶ cells per flask, 100-600×10⁶ cells per flask or 150-500×10⁶ cells per flask. In specific embodiments, over the duration of culture in the flasks, the NK cells of the population of NK cells are cultured at a cell density of 100-3000×10⁶ cells per flask.

Culturing the NK cells can be effected with or without feeder cells or a feeder cell layer. According to one embodiment, feeder cells comprise T cells or peripheral blood mononuclear cells (PBMCs). According to a specific embodiment, feeder cells comprise irradiated cells (i.e. non-proliferating cells), e.g. irradiated T cells or irradiated peripheral blood mononuclear cells. Irradiation can be affected, for example, at 20-50 Gy (e.g. 20 Gy, 30 Gy, 40 Gy, 50 Gy), 130 KV, 5 mA. According to one embodiment, when feeder cells are used, the ratio of NK cells to feeder cells in the culture may be 1:1, 1:2, 1:3, 2:1 or 3:1. According to a specific embodiment, the ratio of NK cells to feeder cells in the culture is 1:1.

According to a specific embodiment, when T cells or PBMCs (e.g. irradiated T cells or irradiated PBMCs) are used as feeder cells, the culture is further supplemented with a CD3 agonist to stimulate the T-cells in the feeder cell layer to secrete growth factors beneficial for NK cell expansion. CD3 agonists suitable for use with the method of some embodiments of the invention include, but are not limited to, anti-CD3 monoclonal-CD3 agonist antibodies such as OKT-3, mAb 145-2C11, MGA031 and ChAglyCD3.

According to one embodiment, the method comprises upregulating expression of CAR or a tg-TCR capable of binding HER2 in the ex vivo expanded population of NK cells.

The terms “HER2”, “ERBB2” or “HER-2/neu” as used herein interchangeably refer to the gene product of the erb-b2 receptor tyrosine kinase 2 (ERBB2) gene having the gene symbol “ERBB2”, or for example, GeneBank Accession nos. NP_001005862.1, NP_001276865.1 and NP_001276866.1 (protein) and NM_001005862.3, NM_001289936.2 and NM_001289937.2 (mRNA), or homologs thereof.

HER2 polypeptide is a member of the epidermal growth factor receptor (EGFR) family of receptor tyrosine kinases. HER2 polypeptide typically binds to other EGFR family members to form a heterodimer, stabilizing ligand binding and enhancing kinase-mediated activation of downstream signaling pathways, such as those involving mitogen-activated protein kinase and phosphatidylinositol-3 kinase.

As used herein the phrase “upregulating expression” refers to increasing the expression of a CAR or a tg-TCR on NK cells. The CAR or tg-TCR of some embodiments of the invention is not naturally expressed by the NK cells (i.e. is an exogenous protein).

For the same culture conditions the expression is generally expressed in comparison to the expression in a cell of the same species but not modified to increase the level of mRNA and/or protein of a CAR or a tg-TCR, or contacted with a vehicle control, also referred to as “control”.

According to one embodiments upregulating the expression of CAR or a tg-TCR refers to increasing the level of mRNA and/or protein, as detected by RT-PCR or Western blot, respectively. The increase may be by at least a 10%, at least 20%, at least 30%, at least 40%, at least 50%, at least 60%, at least 70%, at least 80%, at least 90%, at least 95% or by at least 99% or more.

Upregulation the expression of a CAR or a tg-TCR is typically affected at the transcript level or at the protein level. According to a specific embodiment, upregulation of the expression of a CAR or a tg-TCR on NK cells is affected by introducing exogenous nucleic acids (e.g. mRNA) encoding the CAR or tg-TCR into NK cells. Thus, the NK cells of some embodiments of the invention are modified to express the CAR or tg-TCR.

Upregulation of expression may be either transient or permanent. According to a specific embodiment, the expression of a CAR or a tg-TCR is transient (i.e. the cells are not genetically modified in their genome for expression of the CAR or tg-TCR).

As used herein, the term “transgenic T cell receptor” or “tg-TCR” refers to a recombinant molecule comprising the specificity of a T cell receptor (TCR), i.e. recognition of antigenic peptides (i.e. antigens) presented by major histocompatability complex (MHC) proteins. Typically, the TCR recognizes antigens, i.e. peptides of foreign (e.g. viral) or cellular (e.g. tumor) origins which have been processed by the cell, loaded onto the MHC complex and trafficked to the cell membrane as a peptide-MHC complex.

The tg-TCR of the invention typically comprises two chains (i.e., polypeptide chains), such as, an alpha chain of a T cell receptor (TCR), a beta chain of a TCR, a gamma chain of a TCR, a delta chain of a TCR, or a combination thereof (e.g. αβ chains or γδ chains). The polypeptides of the tg-TCR can comprise any amino acid sequence, provided that the tg-TCR has antigenic specificity and T cell effector functions as described hereinabove. It will be appreciated that antigen specificity is determined by the TCR heterodimer (i.e. by the αβ or γδ chains).

It will be appreciated that each of the two chains is typically composed of two extracellular domains, i.e. the variable (V) region and the constant (C) region.

According to one embodiment, the tg-TCR comprises the variable regions of a TCR. According to a specific embodiment, the tg-TCR comprises the variable regions of α- and β-chains of a TCR. According to another specific embodiment, the tg-TCR comprises the variable regions of γ- and δ-chains of a TCR.

According to some embodiments of the invention, the variable region of the tg-TCR comprises complementarity determining regions (CDRs) which are capable of specifically binding the antigen. The CDRs may be selected from any of CDR1, CDR2, CDR3 and/or CDR4. According to a specific embodiment, the CDRs are present on a single chain, preferably the CDRs are present on both chains of the tg-TCR.

According to one embodiment, the tg-TCR comprises the constant regions of a TCR. According to a specific embodiment, the tg-TCR comprises the constant regions of α- and β-chains of a TCR. According to another specific embodiment, the tg-TCR comprises the constant regions of γ- and δ-chains of a TCR.

The choice of tg-TCR depends upon the type and number of antigens that define the MHC-peptide complex of a target cell. For example, the tg-TCR may be chosen to recognize an MHC-peptide complex on a target cell associated with a particular disease state. Thus, for example, markers that may act as antigens for recognition by the tg-TCR may include those associated with viral, bacterial and parasitic infections and cancer cells. Examples are provided below.

To generate a successful tg-TCR, an appropriate target sequence needs to first be identified. Accordingly, a TCR may be isolated from an antigen reactive T cell (e.g. tumor reactive T cell) or, where this is not possible, alternative technologies can be employed. According to an exemplary embodiment, a transgenic animal (e.g. rabbit or mouse, preferably a human-HLA transgenic mouse) is immunized with human antigen peptides (e.g. tumor or viral antigens) to generate T cells expressing TCRs against the human antigens [as described e.g. in Stanislawski et al., Nat Immunol. (2001) 2(10):962-70]. According to another exemplary embodiment, antigen-specific T cells (e.g. tumor specific T cells) are isolated from a patient experiencing disease (e.g. tumor) remission and the reactive TCR sequences are isolated therefrom [as described e.g. in de Witte et al., Blood (2006) 108(3):870].

According to another exemplary embodiment, in vitro technologies are employed to alter the sequence of an existing TCR to enhance the avidity of a weakly reactive antigen-specific TCR with a target antigen (such methods are described below).

According to one embodiment, the signaling module of the tg-TCR may comprise a single subunit or a plurality of signaling units. Accordingly, the tg-TCR of the invention may use co-receptors that act in concert with a TCR to initiate signal transduction following antigen receptor engagement, as well as any derivative or variant of thereof having the same functional capability.

According to one embodiment, the TCR signaling module comprises the CD3 complex (e.g. CD3 chains, e.g. CD3δ/ε, CD3γ/ε and/or zeta chains, e.g. ζ/ζ or ζ/η).

Additionally or alternatively, the TCR signaling module may comprise co-stimulatory domains to provide additional signals to the T cell. These are discussed in detail for CAR molecules herein below.

According to one embodiment, the tg-TCR may comprise a transmembrane domain as described in detail for CAR molecules herein below.

As used herein the phrase “chimeric antigen receptor (CAR)” refers to a recombinant molecule which combines specificity for a desired antigen (i.e. HER2) with a T cell receptor-activating intracellular domain (i.e. T cell receptor signaling module) to generate a chimeric protein that exhibits cellular immune activity to the specific antigen. Typically, a CAR recognizes an antigen (e.g. protein or non-protein) expressed on the cell surface (rather than internal antigens) independently of the major histocompatibility complex (MHC).

Thus, the CAR of the invention generally comprises an extracellular domain comprising an antigen binding moiety, a transmembrane domain and an intracellular domain (i.e. the cytoplasmic domain also referred to as endo-domain) that is required for an efficient response of the T cell to the antigen.

### Antigen Binding Moiety

In one embodiment, the CAR of the invention comprises a target-specific binding element otherwise referred to as an antigen binding moiety. The choice of moiety depends upon the type and number of ligands (i.e. antigens) that define the surface of a target cell. For example, the antigen binding domain may be chosen to recognize a ligand (i.e. antigen) that acts as a cell surface marker on target cells associated with a particular disease state. Thus examples of cell surface markers that may act as ligands for the antigen moiety domain in the CAR of the invention include those associated with viral, bacterial and parasitic infections and cancer cells.

According to some embodiments of the invention, the antigen binding moiety comprises complementarity determining regions (CDRs) which are capable of specifically binding the antigen. Such CDRs can be obtained from an antibody.

The term “antibody” as used in this invention includes intact molecules as well as functional fragments thereof, such as Fab, Fab′, F(ab′)2, Fv, linear antibodies, scFv antibodies, and multispecific antibodies formed from antibody fragments that are capable of binding to the antigen. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; (6) CDR peptide is a peptide coding for a single complementarity-determining region (CDR); and (7) Single domain antibodies (also called nanobodies), a genetically engineered single monomeric variable antibody domain which selectively binds to a specific antigen. Nanobodies have a molecular weight of only 12-15 kDa, which is much smaller than a common antibody (150-160 kDa).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa- and lambda-light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Once the CDRs of an antibody are identified, using conventional genetic engineering techniques, expressible polynucleotides encoding any of the forms or fragments of antibodies described herein can be synthesized and modified in one of many ways in order to produce a spectrum of related-products.

According to some embodiments of the invention, the CDRs are derived from αβ T cell receptor (TCR) which specifically binds to the antigen.

According to some embodiments of the invention, the CDRs are derived from γδ T cell receptor (TCR) which specifically binds to the antigen.

According to some embodiments of the invention, the CDRs are derived from an engineered affinity-enhanced αβ T cell receptor or γδ T cell receptor (TCR) which specifically binds to the antigen (as discussed in detail herein above).

According to some embodiments of the invention, the CDRs are derived from an engineered αβ T cell receptor or γδ T cell receptor (TCR) with improved stability or any other biophysical property.

According to some embodiments of the invention, the CDRs are derived from a T cell receptor-like (TCRLs) antibody which specifically binds to the antigen. Examples of TCRLs and methods of generating same are described in WO03/068201, WO2008/120203, WO2012/007950, WO2009125395, WO2009/125394, each of which is fully incorporated herein by their entirety.

According to some embodiments of the invention, the antigen binding domain comprises one or more single chain Fv (scFv) molecule.

### Cytoplasmic Domain

The cytoplasmic domain (also referred to as “intracellular signaling domain” or “T cell receptor signaling module”) of the CAR molecule of the invention is responsible for activation of at least one of the normal effector functions of the cell in which the CAR has been placed in.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the CAR molecule of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

Thus, NK cell activation can be mediated by two distinct classes of cytoplasmic signaling sequence: those that initiate antigen-dependent primary activation (primary cytoplasmic signaling sequences) and those that act in an antigen-independent manner to provide a secondary or co-stimulatory signal (secondary cytoplasmic signaling sequences).

Primary cytoplasmic signaling sequences that act in a stimulatory manner may contain signaling motifs which are known as immunoreceptor tyrosine-based activation motifs (ITAMs). Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the CAR of the invention comprises a cytoplasmic signaling sequence derived from CD3 zeta.

The co-stimulatory signaling region typically refers to a portion of the CAR molecule comprising the intracellular domain of a co-stimulatory molecule. Co-stimulatory molecules are cell surface molecules other than antigen receptors or their ligands that are required for an efficient response of lymphocytes to antigen. Co-stimulatory molecules include but are not limited to an MHC class I molecule, BTLA and a Toll ligand receptor. A co-stimulatory ligand can include, but is not limited to, CD7, B7-1 (CD80), B7-2 (CD86), PD-L1, PD-L2, 4-1BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

According to one embodiment, the cytoplasmic domain of the CAR can be designed to comprise the CD3-zeta signaling domain by itself or combined with any other desired cytoplasmic domain(s) useful in the context of the CAR of the invention. For example, the cytoplasmic domain of the CAR can comprise a CD3 zeta chain portion and a co-stimulatory signaling region. The co-stimulatory signaling region refers to a portion of the CAR comprising the intracellular domain of a co-stimulatory molecule. A co-stimulatory molecule is a cell surface molecule other than an antigen receptor or their ligands that is required for an efficient response of lymphocytes to an antigen. Examples of such molecules include CD27, CD28, 4-1BB (CD137), OX40 (CD134), CD30, CD40, PD-1, DAP10, 2B4, Lsk, ICOS, lymphocyte function-associated antigen-1 (LFA-1), CD2, CD7, LIGHT, NKG2C, B7-H3, and a ligand that specifically binds with CD83, and the like.

According to some embodiments of the invention, the intracellular domain comprises the CD3ζ-chain [CD247 molecule, also known as “CD3-ZETA” and “CD3z”; GenBank Accession NOs. NP_000725.1 and NP_932170.1], which is the primary transmitter of signals from endogenous TCRs.

According to some embodiments of the invention, the intracellular domain comprises various co-stimulatory protein receptors to the cytoplasmic tail of the CAR to provide additional signals to the T cell (“second generation” CAR). Examples include, but are not limited to, CD28 [e.g., GenBank Accession Nos. NP_001230006.1, NP_001230007.1, NP_006130.1], 4-1BB [tumor necrosis factor receptor superfamily, member 9 (TNFRSF9), also known as “CD137”, e.g., GenBank Accession No. NP_001552.2], ICOS [inducible T-cell co-stimulator, e.g., GenBank Accession No. NP_036224.1], DAP10 [hematopoietic cell signal transducer, e.g., GenBank Accession Nos. NP_001007470, NP_055081.1], 2B4 [CD244 molecule, e.g. GenBank Accession Nos. NP_001160135.1, NP_001160136.1, NP_057466.1] and Lsk [LCK proto-oncogene, Src family tyrosine kinase, e.g., GenBank Accession Nos. NP_001036236.1, NP_005347.3]. Preclinical studies have indicated that the “second generation of CAR designs improves the antitumor activity of T cells.

According to some embodiments of the invention, the intracellular domain comprises at least one, at least two, at least three or more of the polypeptides selected from the group consisting of: CD3ζ (CD247, CD3z), CD27, CD28, 4-1BB/CD137, 2B4, ICOS, OX40/CD134, DAP10, tumor necrosis factor receptor (TNFr) and Lsk.

According to some embodiments of the invention, the intracellular domain comprises multiple signaling domains, such as CD3z-CD28-4-1BB or CD3z-CD28-OX40, to further augment potency. The term “OX40” refers to the tumor necrosis factor receptor superfamily, member 4 (TNFRSF4), e.g., GenBank Accession No. NP_003318.1 (“third-generation” CARs).

According to some embodiments of the invention, the intracellular domain comprises CD28-CD3z, CD3z, CD28-CD137-CD3z. The term “CD137” refers to tumor necrosis factor receptor superfamily, member 9 (TNFRSF9), e.g., GenBank Accession No. NP_001552.2.

According to a specific embodiment, the intracellular domain comprises CD3z and CD28.

According to a specific embodiment, the intracellular domain comprises CD3z and 4-1BB.

According to a specific embodiment, the intracellular domain comprises CD3z and 2B4.

### Transmembrane Domain

The transmembrane domain of the CAR may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154 or NKG2D. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain.

According to a specific embodiment, the transmembrane domain comprises CD8.

According to a specific embodiment, the transmembrane domain comprises CD28.

According to a specific embodiment, the transmembrane domain comprises NKG2D.

According to some embodiments of the invention, the transmembrane domain comprised in the CAR molecule of some embodiments of the invention is a transmembrane domain that is naturally associated with one of the domains in the CAR. According to some embodiments of the invention, the transmembrane domain can be selected or modified by amino acid substitution to avoid binding of such domains to the transmembrane domains of the same or different surface membrane proteins to minimize interactions with other members of the receptor complex.

According to some embodiments, between the extracellular domain and the transmembrane domain of the CAR molecule, or between the cytoplasmic domain and the transmembrane domain of the CAR molecule, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

Optionally, a short oligo- or polypeptide linker, preferably between 2 and 10 amino acids in length may form the linkage between the transmembrane domain and the cytoplasmic signaling domain of the CAR (also referred to as “hinge”). A glycine-serine doublet provides a particularly suitable linker.

According to a specific embodiment, a hinge region of CD8 is used in construction of the CAR molecule.

According to a specific embodiment, a hinge region of CD28 is used in construction of the CAR molecule.

According to a specific embodiment, the CAR of some embodiments of the invention is GDA-501.A, as set forth in SEQ ID Nos: 23-24 (nucleic acid and amino acid sequences, respectively).

According to a specific embodiment, the CAR of some embodiments of the invention is GDA-501.B, as set forth in SEQ ID Nos: 25-26 (nucleic acid and amino acid sequences, respectively).

According to a specific embodiment, the CAR of some embodiments of the invention is GDA-501.C, as set forth in SEQ ID Nos: 27-28 (nucleic acid and amino acid sequences, respectively).

According to a specific embodiment, the CAR of some embodiments of the invention is GDA-501.D, as set forth in SEQ ID Nos: 29-30 (nucleic acid and amino acid sequences, respectively).

As mentioned, the CAR or tg-TCR has antigenic specificity for the tumor antigen HER2.

As used herein the phrase “tumor antigen” refers to an antigen that is common to specific hyperproliferative disorders such as cancer. Tumor antigens are proteins that are produced by tumor cells that elicit an immune response, particularly T-cell mediated immune responses. The selection of the antigen binding moiety of the invention will depend on the particular type of cancer to be treated.

The type of tumor antigen referred to in the invention includes a tumor-specific antigen (TSA) or a tumor-associated antigen (TAA). A “TSA” refers to a protein or polypeptide antigen unique to tumor cells and which does not occur on other cells in the body. A “TAA” refers to a protein or polypeptide antigen that is expressed by a tumor cell. For example, a TAA may be one or more surface proteins or polypeptides, nuclear proteins or glycoproteins, or fragments thereof, of a tumor cell.

According to one embodiment, HER2 is associated with a solid tumor.

Various methods can be used to introduce nucleic acids of some embodiments of the invention into NK cells (e.g. nucleic acids encoding a CAR or a tg-TCR). Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

According to one example, nucleic acids of some embodiments of the invention are introduced into NK cells as naked DNA or in a suitable vector. Methods of stably transfecting cells by electroporation using naked DNA are known in the art. See, e.g., U.S. Pat. No. 6,410,319. Naked DNA generally refers to the DNA encoding a CAR or a tg-TCR contained in a plasmid expression vector in proper orientation for expression.

Alternatively, a viral vector (e.g., a retroviral vector, adenoviral vector, adeno-associated viral vector, or lentiviral vector) can be used to introduce the nucleic acids of some embodiments of the invention into NK cells (e.g. nucleic acids encoding a CAR or a tg-TCR). Suitable vectors for use in accordance with the method of the present disclosure are non-replicating in the NK cells. A large number of vectors are known that are based on viruses, where the copy number of the virus maintained in the cell is low enough to maintain the viability of the cell, such as, for example, vectors based on HIV, SV40, EBV, HSV, or BPV.

According to one example, nucleic acids of some embodiments of the invention are introduced into NK cells by non-viral gene transfer.

According to one example, nucleic acids of some embodiments of the invention are introduced into NK cells as mRNA.

According to a specific embodiment, upregulating the expression of a CAR or a tg-TCR is affected by electroporation of nucleic acids (e.g. mRNA) into the NK cells. Electroporation may be affected using any electroporation device, such as but not limited to, a Nucleofector or BTX-Gemini Twin Wave Electroporator.

According to one embodiment, upregulating the expression of a CAR or a tg-TCR is affected 8-20 days, 8-18 days, 10-18 days, 12-18 days, 12-16 days, 12-14 days from initiation of the cell culture.

According to a specific embodiment, upregulating the expression of a CAR or a tg-TCR is affected 12-16 days from initiation of the cell culture.

According to a specific embodiment, upregulating the expression of a CAR or a tg-TCR is affected 12-14 days from initiation of the cell culture.

In certain embodiments, after the NK cells have been modified to express CAR or a tg-TCR, the cells may be harvested from the culture.

According to a specific embodiment, the cells are modified to express a CAR or a tg-TCR 1-4 days, 1-3 days, 1-2 days or 0.5-1 day prior to harvesting of the cells.

Harvesting of the cells can be performed manually, by releasing attached cells (e.g. “scraping” culture vessel surfaces) or by a cell harvesting device, which is designed to efficiently wash cells out of their culture vessels and collect the cells automatically. In specific embodiments, the expanded NK cells are harvested from the culture vessels by a cell harvesting device (e.g. the harvesting device of the G-Rex MCS, WolfWilson, St Paul MN). In specific embodiments, the expanded CD3-depleted NK cell fraction is harvested from the culture vessels by a cell harvesting device (e.g. the LOVO Cell Processing device by Fresenius Kabi (Hamburg, Germany)).

In some embodiments, harvesting of expanded NK cells from culture removes most, or nearly all of the cells from the culture vessel. In other embodiments, harvesting can be performed in two or more steps, allowing the unharvested cells to remain in culture until harvested at a later time. In certain embodiments, the expanded NK cells are harvested in two steps, comprising harvesting a first portion of the expanded NK cells, and then harvesting a second portion of the expanded NK cells. Harvesting the two portions can be performed with an interval of hours, days or more between harvesting of the first and second portion. The two portions harvested can comprise approximately equal portions of the culture (e.g. equal amounts of the cultured NK cells), or one of the portions may be comprise a larger fraction of the cultured NK cells than the other). According to one embodiment, harvesting comprises harvesting the expanded modified NK cells about 12-18 days, e.g. 14-16 days, following initiation of culture. According to one embodiment, harvesting comprises harvesting the expanded modified NK cells about 1-4 days, e.g. 1-2 days, after modifying the cells to express a CAR or a tg-TCR (e.g. anti-HER2 CAR, anti-HER2 tg-TCR).

In order to prepare the expanded population of NK cells for use, the harvested cells need to be washed of culture medium, critical parameters evaluated and volume adjusted to a concentration suitable for infusion over a clinically reasonable period of time.

Following harvesting, the expanded modified NK cells can be washed free of culture medium manually or, preferably for clinical applications, using an automated device employing a closed system. Washed cells can be reconstituted with an infusion solution (for example, one exemplary infusion solution comprises 8% w/v HSA and 6.8% w/v Dextran-40). In some embodiments, the reconstitution is performed in a closed system. In some embodiments, the infusion solution is screened for suitability for use with the methods and compositions of the present invention. Exemplary criteria for selection of suitable infusion solution include safety tests indicating no bacterial, yeast or mold growth, endotoxin content of less than 0.5 Eu/ml and a clear, foreign particle-free appearance.

Once the expanded modified NK cells are obtained, the cells are examined for the number of cells (i.e. proliferation), for cell signature (e.g. CD3−CD56+ cells), for the expression of the CAR or the tg-TCR (e.g. anti-HER2 CAR, anti-HER2 tg-TCR) and for NK cell functionality.

Assays for cell proliferation are well known in the art, and include without being limited to, clonogenic assays, in which cells are seeded and grown in low densities, and colonies counted, mechanical assays [flow cytometry (e.g., FACS™), propidium iodide], which mechanically measure the number of cells, metabolic assays (such as incorporation of tetrazolium salts e.g., XTT, MTT, etc.), which measure numbers of viable cells, direct proliferation assays (such as bromodeoxyuridine, thymidine incorporation, and the like), which measure DNA synthesis of growing populations.

Assays for cell signature and for expression of proteins on a cell membrane are well known in the art, and include without being limited to, FACS analysis and immunohistological staining techniques.

As used herein, the term “NK cell functionality” refers to any biological function ascribed to NK cells. A non-limiting list of NK cell functions includes, for example, cytotoxicity, induction of apoptosis, cell motility, directed migration, cytokine and other cell signal response, cytokine/chemokine production and secretion, expression of activating and inhibitory cell surface molecules in-vitro, cell homing and engraftment (in vivo retention) in a transplanted host, and alteration of disease or disease processes in vivo. In some embodiments, NK cell functions enhanced by expansion in the presence of nicotinamide and/or other nicotinamide moiety include at least one of elevated expression of CD62L surface marker, elevated migration response, and greater cytotoxic activity of the NK cells, as well as elevated homing and in vivo retention of infused NK cells.

Assays for adhesion and migration molecules such as CD62L, CXCR-4, CD49e and the like, important for homing/engraftment and retention of cells in transplantation, are well known in the art. CD62L expression in a cell can be assayed, for example, by flow cytometry, immunodetection, quantitative cDNA amplification, hybridization and the like.

Assays for cells migration are well known in the art. Migration of cells can be assayed, for example, by transmigration assays or gap closure assays. In one embodiment, migration potential of different populations of NK cells is determined by the “Transwell”™ transmigration assay.

Assays for cytotoxicity (“cell killing”) are well known in the art. Examples of suitable target cells for use in redirected killing assays are cancer cell line, primary cancer cells solid tumor cells, leukemic cells, or virally infected cells. Particularly, K562, BL-2, colo250 and primary leukemic cells can be used, but any of a number of other cell types can be used and are well known in the art (see, e.g., Sivori et al. (1997) J. Exp. Med. 186: 1129-1136; Vitale et al. (1998) J. Exp. Med. 187: 2065-2072; Pessino et al. (1998) J. Exp. Med. 188: 953-960; Neri et al. (2001) Clin. Diag. Lab. Immun. 8: 1131-1135). For example, cell killing may be assessed by cell viability assays (e.g., dye exclusion, chromium release, CFSE), metabolic assays (e.g., tetrazolium salts), and direct observation.

The washed and concentrated expanded modified NK cell fraction generated by some embodiments of the invention is characterized by comprising about 60% to about 99% CD56+/CD3− cells, about 70% to about 99% CD56+/CD3− cells, about 80% to about 99% CD56+/CD3− cells or about 90-99% CD56+/CD3− cells. In one embodiment, the washed and concentrated expanded NK cell fraction generated by some embodiments of the invention is characterized by comprising at least about 60%, at least 70%, at least 80%, at least 90%, or at least 95% CD56+/CD3− cells.

The washed and concentrated expanded modified NK cell fraction generated by some embodiments of the invention is characterized by comprising about 60% to about 99% CAR or tg-TCR positive cells, about 70% to about 99% CAR or tg-TCR positive cells, about 80% to about 99% CAR or tg-TCR positive cells or about 90-99% CAR or tg-TCR positive cells (e.g. anti-HER2 CAR, anti-HER2 tg-TCR). In one embodiment, the washed and concentrated expanded NK cell fraction generated by some embodiments of the invention is characterized by comprising at least about 60%, at least 70%, at least 80%, at least 90%, or at least 95% CAR or tg-TCR positive cells (e.g. anti-HER2 CAR, anti-HER2 tg-TCR).

The modified NK cells of some embodiments of the invention may be used as fresh cells. Alternatively, the cells may be cryopreserved for future use, or “off the shelf” use.

According to an aspect of some embodiments of the invention there is provided an isolated population of NK cells obtainable according to the methods of some embodiments of the invention.

According to one embodiment, the isolated population of NK cells (i.e. following ex vivo expansion, e.g. at the end of culture) comprise at least about 50%, 60%, 70%, 75%, 80%, 85%, 90% or 95% or more NK cells.

According to one embodiment, at least about 50%, 60%, 70%, 75%, 80%, 85%, 90% or 95% or more of the isolated population of NK cells (i.e. following ex vivo expansion, e.g. at the end of culture) express CAR or tg-TCR.

According to one embodiment, the isolated population of NK cells (i.e. following ex vivo expansion, e.g. at the end of culture) comprise at least about 50%, 60%, 70%, 75%, 80%, 85%, 90% or 95% or more anti-HER2 NK CAR cells.

According to one embodiment, the isolated population of NK cells (i.e. following ex vivo expansion, e.g. at the end of culture) comprise at least about 50%, 60%, 70%, 75%, 80%, 85%, 90% or 95% or more anti-HER2 NK tg-TCR cells.

The isolated population of NK cells of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the isolated population of NK cells accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences”, Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method. Alternately, one may administer the pharmaceutical composition in a local rather than systemic manner, for example, via injection of the pharmaceutical composition directly into a tissue region of a patient.

According to one embodiment, the route of administration includes, for example, an injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the pharmaceutical composition of the present invention is administered to a patient by intradermal or subcutaneous injection. In another embodiment, the pharmaceutical composition of the present invention is preferably administered by i.v. injection. The pharmaceutical composition may be injected directly into a tumor, lymph node, or site of infection.

Pharmaceutical compositions of some embodiments of the invention may be manufactured by processes well known in the art, e.g., by means of conventional mixing, dissolving, granulating, dragee-making, levitating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion.

Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acids esters such as ethyl oleate, triglycerides or liposomes.

Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water-based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (isolated population of NK cells) effective to prevent, alleviate or ameliorate symptoms of a disorder (e.g., malignant or non-malignant disease) or prolong the survival of the subject being treated.

Determination of a therapeutically effective amount is well within the capability of those skilled in the art, especially in light of the detailed disclosure provided herein.

When “therapeutic amount” is indicated, the precise amount of the compositions of the present invention to be administered can be determined by a physician with consideration of individual differences in age, weight, disease state, e.g. tumor size, extent of infection or metastasis, and the condition of the patient (subject). It can generally be stated that a pharmaceutical composition comprising the cells described herein may be administered at a dosage of 25-500×10⁶ cells per kg body weight, e.g. 25-400×10⁶ cells per kg body weight, 50-300×10⁶ cells per kg body weight, e.g. 50-250×10⁶ cells per kg body weight, including all integer values within those ranges. According to one embodiment, the cells described herein may be administered at a dosage of about 25×10⁶ cells per kg body weight, about 50×10⁶ cells per kg body weight, about 75×10⁶ cells per kg body weight, about 100×10⁶ cells per kg body weight, about 150×10⁶ cells per kg body weight, about 200×10⁶ cells per kg body weight, about 250×10⁶ cells per kg body weight, or about 300×10⁶ cells per kg body weight.

The NK cell compositions of some embodiments of the invention may also be administered multiple times at these dosages. The NK cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

For example, the effect of the active ingredients (e.g., the isolated population of NK cells of some embodiments of the invention) on the pathology can be evaluated by monitoring the level of cellular markers, hormones, glucose, peptides, carbohydrates, cytokines, etc. in a biological sample of the treated subject using well known methods (e.g. ELISA, FACS, etc) or by monitoring the tumor size using well known methods (e.g. ultrasound, CT, MRI, etc).

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved. According to one embodiment, the dosing can be one, two, three or more administrations per day. The dosing can be on subsequent days, or within days or weeks apart. Such determinations can readily be determined by one skilled in the art of medicine.

The amount of a composition to be administered will, of course, be dependent on the subject being treated, the severity of the affliction, the manner of administration, the judgment of the prescribing physician, etc.

According to some embodiments of the invention, the therapeutic agent of the invention can be provided to the subject in conjunction with other drug(s) designed for treating the pathology [i.e. combination therapy, e.g., before, concomitantly with, or following administration of the isolated population of NK cells].

According to one embodiment of the invention, the isolated population of NK cells of some embodiments of the invention may be used in combination with chemotherapy, radiation therapy, immunosuppressive agents (e.g. cyclosporin, azathioprine, methotrexate, mycophenolate, and FK506), antibodies, or other agents known in the art.

In certain embodiments, the isolated population of NK cells of some embodiments of the invention are administered to a patient in conjunction with any number of relevant treatment modalities, including but not limited to treatment with agents such as antiviral agents (e.g. Ganciclovir, Valaciclovir, Acyclovir, Valganciclovir, Foscarnet, Cidofovir, Maribavir, Leflunomide), chemotherapeutic agents (e.g. antineoplastic agents, such as but not limited to, Alkylating agents including e.g. Cyclophosphamide, Busulfan, Mechlorethamine or mustine (HN2), Uramustine or uracil mustard, Melphalan, Chlorambucil, Ifosfamide, Bendamustine, Nitrosoureas Carmustine, Lomustine, Streptozocin, Thiotepa, Cisplatin, Carboplatin, Nedaplatin, Oxaliplatin, Satraplatin, Triplatin tetranitrate, Procarbazine, Altretamine, Triazenes (dacarbazine, mitozolomide, temozolomide), Dacarbazine, Temozolomide, Myleran, Busulfex, Fludarabine, Dimethyl mileran or Cytarabine) or therapeutic monoclonal antibodies (Exemplary antibodies are provided in table 1, below).

TABLE 1
Anti-Cancer Therapeutic Monoclonal Antibodies

| Antibody | Cancer Indication | Target |
|---|---|---|
| Trastuzumab (Herceptin ®) | B/C, gastric, gastrointestinal junction | HER2 |
| | adenocarcinoma | |
| Pertuzumab (Perjeta ®) | HER2+ B/C | HER2 |
| Certuximab (Erbitux ®) | Metastatic CRC, HNSCC | EGFR |
| Panitumumab (Vectibix ®) | Metastatic CRC | EGFR |
| Necitumumab (Portrazza ®) | Metastatic squamous NSCLC | EFGR |
| Dinutuximab (Unituxin ®) | Pediatric neuroblastoma | GD2 |
| Bevacizumab (Avastin ®) | CRC, NSCLC, B/C, Renal Cell Carcinoma | VEGF-A |
| | (RCC), cervical cancer, Glioblastoma, Ovarian, | |
| | Fallopian, primary peritoneal cancer | |
| Ramucirumab (Cyramza ®) | Metastatic gastric, gastroesophageal junction | VEGFR-2 |
| | adenocarcinoma | |
| Olaratumab (Lartruvo ®) | Soft tissue sarcoma | PDGFR-alpha |
| Ipilimumab (Yervoy ®) | Metastatic or cutaneous melanoma, | CTLA-4 |
| Nivolumab (Opdivo ®) | Metastatic melanoma, squamous NSCLC, | PD-1 |
| | NSCLC, RCC, HNSCC | |
| Pembrolizumab | Metastatic melanoma, NSCLC, HNSCC | PD-1 |
| (Keytruda ®) | | |
| Atezolizumab (Tecentriq ®) | Urothelial carcinoma | PD-L1 |
| Ado-trastuzumab emtansine | HER2+ B/C | HER2 |
| (Kadcycla ®) fusion | | |
| Denosumab (Xgeva ®) | Bone metastases | RANKL |
| Alemtuzumab (Campath ®) | CLL | CD52 |
| Avelumab (Bavencio ®) | Merkel cell carcinoma | PD-L1 |
| Blinatumomab (Blincyto ®) | ALL | CD19 |
| Brentuximab vedotin | Hodgkins lymphoma | CD30 |
| (Adcetris ®) | | |
| Capromab pendetide | Prostate | PSMA |
| (ProstaScint ®) | | |
| Daratumumab (Darzalex ®) | Multiple myeloma | CD38 |
| Durvalumab (Imfinzi ®) | Urothelial carcinoma | PD-L1 |
| Elotuzumab (Empliciti ®) | Multiple myeloma | SLAMF7 |
| Ibritumomab tiuxetan | Non-Hodgkins lymphoma | CD20 |
| (Zevalin ®) | | |
| Obinutuzumab (Gazyva ®) | CLL | CD20 |
| Ofatumumab (Arzerra ®) | CLL | CD20 |
| Pertuzumab (Perjeta ®) | Metastatic B/C | HER2 |
| Rituximab (Rituxan ®) | B cell Non-Hodgkins lymphoma | CD20 |
| Rituximab-hyaluronidase | CLL, B-cell lymphoma | CD20 |
| (Rituxan Hycela ®) | | |
| Inotuzumab ozogamicin | ALL | CD22 |
| (Besponsa ®) | | |
| Bevacizumab-awwb | CRC, NSCLC, Glioblastoma, RCC, Cervical | VEGF |
| (Mvasi ®) | cancer | |
| Trastuzumab dkst | B/C, gastric, gastroesophageal | HER2 |
| (Ogivri ®) | | |
| Tositumomab (Bexxar ®) | NHL | |

RCC: Renal Cell Carcinoma; ALL: Acute Lymphoblastic Leukemia; CLL: Chronic Lymphocytic Leukemia; NSCLC: Non-Small Cell Lung Cancer; HNSCC: Head and neck squamous cell carcinoma; B/C: Breast Cancer; CRC: Colorectal cancer, NHL: Non-Hodgkin's Lymphoma.

In a specific embodiment, the isolated population of NK cells of some embodiments of the invention are administered to a patient in conjunction with Trastuzumab (Herceptin®, Herceptin Hylecta®), Pertuzumab (Perjeta®), Margetuximab (Margenza®), Ado-trastuzumab emtansine (Kadcyla® or TDM-1®), Fam-trastuzumab deruxtecan (Enhertu®), Lapatinib (Tykerb®), Neratinib (Nerlynx®), Tucatinib (Tukysa®) or a combination thereof.

In a specific embodiment, the isolated population of NK cells of some embodiments of the invention are administered to a patient in conjunction with Trastuzumab (e.g. Herceptin®, Herceptin Hylecta®).

In a specific embodiment, the isolated population of NK cells of some embodiments of the invention are administered to a patient in conjunction with Pertuzumab (Perjeta®).

In a specific embodiment, the isolated population of NK cells of some embodiments of the invention are administered to a patient in conjunction with Ado-trastuzumab emtansine (Kadcyla® or TDM-1®).

In a specific embodiment, the isolated population of NK cells of some embodiments of the invention are administered to a patient in conjunction with Neratinib (Nerlynx®), In a specific embodiment, the isolated population of NK cells of some embodiments of the invention are administered to a patient in conjunction with Trastuzumab dkst (Ogivri®).

In a specific embodiment, the isolated population of NK cells of some embodiments of the invention are administered to a patient in conjunction with Rituximab®.

It will be appreciated that the isolated population of NK cells of some embodiments of the invention may be administered to a patient in conjunction with a chemotherapeutic agent, radiation therapy, antibody therapy, surgery, phototherapy, etc. The combination therapy may increase the therapeutic effect of the agent of the invention in the treated subject.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The kit may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to an aspect of some embodiments of the invention, there is provided a method of treating a disease associated with expression of HER2 in a subject in need thereof, the method comprising administering to the subject a therapeutically effective amount of the isolated population NK cells of some embodiments of the invention, thereby treating the subject.

According to an aspect of some embodiments of the invention, there is provided a therapeutically effective amount of the isolated population of NK cells of some embodiments of the invention for use in treating a disease associated with expression of HER2 in a subject in need thereof.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” or “subject in need thereof” refers to a mammal, preferably a human being, male or female at any age or gender that suffers from a disease which may be treated with the NK cells.

Thus, the method of the present invention may be applied to treat any disease associated with expression of HER2 such as, but not limited to, a malignant disease (e.g. cancer, e.g. HER2+ cancer cells).

According to one embodiment, the subject has a malignant disease.

### Cancerous Diseases

Malignant diseases (also termed cancers) which can be treated by the method of some embodiments of the invention can be any solid or non-solid tumor and/or tumor metastasis.

Examples of cancer include, but are not limited to, carcinoma, lymphoma, blastoma, sarcoma, and leukemia. More particular examples of such cancers include squamous cell cancer, soft-tissue sarcoma, Kaposi's sarcoma, melanoma, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, rectal cancer, endometrial or uterus cancer e.g. uterine carcinoma, carcinoid carcinoma, salivary gland carcinoma, kidney or renal cancer, liver cancer, prostate cancer, vulval cancer, thyroid cancer, hepatic carcinoma, mesothelioma, a myeloma e.g. multiple myeloma, post-transplant lymphoproliferative disorder (PTLD), neuroblastoma, esophageal cancer, synovial cell cancer, glioma and various types of head and neck cancer (e.g. brain tumor). The cancerous conditions amenable for treatment of the invention include metastatic cancers.

According to one embodiment, the malignant disease is a hematological malignancy. Exemplary hematological malignancies include, but are not limited to, leukemia [e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia, T-cell acute lymphocytic leukemia (T-ALL) and B-cell chronic lymphocytic leukemia (B-CLL)] and lymphoma [e.g., Hodgkin's disease, non-Hodgkin's lymphoma, Burkitt, cutaneous T cell, histiocytic, lymphoblastic, T cell, thymic, B cell, including low grade/follicular; small lymphocytic (SL) NHL; intermediate grade/follicular NHL; intermediate grade diffuse NHL; high grade immunoblastic NHL; high grade lymphoblastic NHL; high-grade small non-cleaved cell NHL; bulky disease NHL; mantle cell lymphoma; AIDS-related lymphoma; and Waldenstrom's Macroglobulinemia].

According to some embodiments of the invention, the pathology is a solid tumor.

According to some embodiments of the invention, the pathology is a tumor metastasis.

According to a specific embodiment, the malignant disease is a breast cancer, a gastric cancer, a gastroesophageal cancer, an oesophageal cancer, an ovarian cancer, an endometrial cancer, a lung cancer, an urothelial cancer or a bladder cancer.

According to some embodiments of the invention, the pathology is a hematological malignancy.

According to a specific embodiment, the malignant disease is leukemia or a lymphoma.

According to a specific embodiment, the malignant disease is a multiple myeloma.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

Ex Vivo Cultures with T Cells as Feeder Cells

On day 0, blood cells were collected by apheresis from a healthy donor. Red blood cells (RBC) were lysed by washing with ACK buffer (Gibco, Dublin, Ireland). CD3+ cells were depleted using CliniMACS and CD3 reagent (Miltenyi Biotec, Germany) according to the manufacturer's instructions.

CD3-depleted cells were washed by CliniMACS buffer with 20% HSA and resuspended in complete MEMα media.

Cells were seeded in MEMα medium containing 0.05 mg/ml Gentamicin (Braun), 2 mM L-glutamine (HyClone), and further supplemented with 10% human AB serum (Gemini), 7 mM nicotinamide (Vertillus) and 20 ng/ml IL-15 (Miltenyi).

0.35×10⁶ cells/ml of CD3-depleted cells were seeded in a GREX100MCS cell culture flask (Wilson Wolf) containing 400 mL MEMα medium and further comprising irradiated CD3+ cells as feeder cells (i.e. irradiated at 40 Gy, 130 KV, 5 mA) at a ratio of 1:1, and 10 ng/ml OKT-3 (Miltenyi). Cells were incubated at 5% CO₂ and 37° C., humidified incubator.

On day 6-9, 400 mL of MEMα medium was added to each G-REX100MCS culture flasks to double the volume.

On day 12-14, cells were counted and prepared for mRNA electroporation for transient expression a chimeric antigen receptor (CAR) targeting HER2, as described below. After electroporation, cells were transferred to a 24-well plate with human-serum-enriched MEMα medium as described above. Cells were recovered for 24-48 hours and then analyzed.

Ex Vivo Cultures with PBMCs as Feeder Cells

On day 0, blood cells were collected by apheresis from a healthy donor. Red blood cells (RBC) were lysed by washing with ACK buffer (Gibco, Dublin, Ireland). CD56+ cells were positively selected using CD56 MicroBeads and LS Column, according manufacturer's instructions (Miltenyi Biotec; Cat. No. 130-050-401 and Cat. No. 130-042-401, respectively). Alternatively, CD56+ cells are selected by negative selection using a mix of MicroBeads (Miltenyi)

CD56+ cells were washed by CliniMACS buffer with 20% HSA resuspended in medium supplemented with 10% human serum and 50 ng/mL IL-2, and seeded in flasks in a concentration of 2×10⁶ cells/ml.

Cells were seeded in MEMα medium containing 0.05 mg/ml Gentamicin (Braun), 2 mM L-glutamine (HyClone), and further supplemented with 10% human AB serum (Gemini), 7 mM nicotinamide (Vertillus) and 20 ng/ml IL-15 (Miltenyi).

4×10⁶ cells/ml of CD56+ cells were seeded in a 6-well Grex culture flask (Wilson Wolf) containing 16 mL MEMα medium and further comprising irradiated peripheral blood mononuclear cells (PBMCs) (fresh or thawed) as feeder cells (i.e. irradiated at 40 Gy, 130 KV, 5 mA) at a ratio of 1:1, and 10 ng/ml OKT-3 (Miltenyi). Cells were incubated at 5% CO₂ and 37° C., humidified incubator.

On day 6-9, 16 mL of MEMα medium was added to each 6-well Grex culture flasks to double the volume.

On day 12-14, cells were counted and prepared for mRNA electroporation for transient expression a chimeric antigen receptor (CAR) targeting HER2, as described below. After electroporation, cells were transferred to a 24-well plate with human-serum-enriched MEMα medium as described above. Cells were recovered for 24-48 hours and then analyzed.

mRNA Electroporation for Transient Protein Expression

At days 12-14 of culture, cells were counted, washed with PBSx1, and then washed again with cold Opti-MEM™ (Gibco™).

For mRNA electroporation, 2×10⁶-4×10⁶ cells, and 10-30 μg mRNA at a final volume of 100 μl, were used. The electroporation was performed in 2 mm cold cuvette in a maximum volume of 400 μl (scaling up per the amounts above), using BTX-Gemini Twin Wave Electroporator at a calibrated program (at voltage 300, duration 1 msc, 1 pulse of square wave).

TABLE 2
list of sequences for mRNA expression

| CAR type | SEQ ID NO: |
|---|---|
| anti-Her2 CAR | 23-24 |
| GDA-501.A | |
| anti-Her2 CAR | 25-26 |
| GDA-501.B | |
| anti-Her2 CAR | 27-28 |
| GDA-501.C | |
| anti-Her2 CAR | 29-30 |
| GDA-501.D | |
| Anti-Her CAR | 31-32 |
| GDA 501.1g | |
| Anti-Her CAR | 33-34 |
| GDA 501.2g | |
| Anti-Her CAR | 35-36 |
| GDA 501.3g | |
| Anti-Her CAR | 37-38 |
| GDA 501.4g | |

Following the electroporation, cells were transferred to 12-well plate with human-serum-enriched MEMα medium as described above. Cells were recovered for 24 hours and then analyzed.

### FACS Analysis

For FACS analysis cells were stained with the following fluorescent antibodies:

TABLE 3
List of antibodies for FACS

| Antibody | Catalog No. |
|---|---|
| CD56 VioBright B515 (FITC) | Miltenyi 130-114-552 |
| CD340 (eRBb-2) (APC) | Miltenyi 130-124-467 |
| CD38 (APC) | Miltenyi 130-113-429 |
| Viability- Helix NP Blue | Biolegend 425305 |

### Sandwich Flow Cytometry Technique

This method was used to determined CAR expression on NK cells. The term ‘sandwich’ was used at the method follows the following order: At the bottom—the CAR-expressing NK cells; in the middle—the protein bound by the CAR; and on the top—The AB conjugate to the epitope on the protein.

For example, to detect anti-Her2 CAR expression on NK cell surface, the NK cells were cultured with 1 μg of Her2 soluble protein for 30 minutes, washed, and then an anti-Her2 APC antibody was added and cultured with the cells for 15 minutes.

### Potency Assay (Intracellular Staining of Proinflammatory Cytokines and CD107α Degranulation Marker)

Potency assay analyzes the expression of various activation markers both intracellular and surface expressed. Selected markers were both indicators of direct cellular cytotoxicity and secretion of pro-inflammatory cytokines capable of promoting the anti-tumoral activity of NK cells.

One of the mechanisms in which the NK cells kill its target is through the release of cytotoxic molecules from lytic granules. This process involves the fusion of the granule membrane with the cytoplasmic membrane of the NK cell, resulting in surface exposure of lysosomal-associated proteins that are typically present on the lipid bilayer surrounding lytic granules, such as CD107a. Therefore, membrane expression of CD107a constitutes a marker of immune cell activation and cytotoxic degranulation. Another killing mechanism the NK cells possess is through the death receptor-induced target cell apoptosis. Activated NK cells secrete a wide variety of cytokines such as IFN-γ and TNFα, GM-CSF and more. IFN-γ is one of the most potent effector cytokines secreted by NK cells and plays a crucial role in antitumor activity. IFN-γ has been shown to modulate caspase, FasL, and TRAIL expression and activates antitumor immunity. As such the potency of the NK cells was evaluated based on the expression of CD107a, TNFα and IFN-γ.

1×10⁶ NK cells were co-cultured with 0.5×10⁶ target cells (K562, RAJI)^{+/-} RTX (0.5 μg/ml) and 2 μl of CD107a antibody was added in a total volume of 1 ml NK medium (MEMα+10% AB serum) in a FACS tube. The controls were prepared as follows; positive control: NK cells+5 μl PMA (50 ng/ml)+1 μl Ionomycin (1 μg/ml), negative control: NK cellss (No target) and the size control: NK, K562, RAJI, NK+K562, NK+RAJI. The cells were centrifuged for 30 sec at 300 rpm and incubated at 37° C. for 30 minutes. After the incubation, BFA and Monensin/GolgiStop (5 μg/ml final conc' BFA, 4 μl GS) were added to each tube. The cells were centrifuged for 30 sec at 300 rpm and incubated at 37° C. for 3.5 hours after which the Zombie viability dye was added and washed.

Cells were stained first for cell surface markers as follows: 1.5 μl of the outer membrane antibody (CD56, CD16) was added and incubated for 10 minutes in the dark in 2-8° C. and washed. The Inside Stain Kit (Miltenyi, CAT #130-090-4777) was used and added for intracellular staining at this point. Cells were fixed and permeabilized, following centrifugation intracellular mAbs were added (IFN-γ and TNF-α) and the cells were incubated for 15 min at room temperature in the dark. The cells were then washed and analyzed.

TABLE 4
Exemplary list of antibodies for potency assay and degranulation

| | | Vendor and |
|---|---|---|
| Antibody | Fluorochrome | Catalog No. |
| Zombie Violet | B.V 421 | Bio Legend, |
| Viability Dye | | Cat. No. 4253 |
| CD107a | VioGreen | Miltenyi |
| | | Cat. No. 130-111-629 |
| CD56 | FITC | Miltenyi |
| | | Cat. No. 130-114-549 |
| TNF-α | PE | Miltenyi |
| | | Cat. No. 130-118-974 |
| IFN-γ | PE-Vio770 | Miltenyi |
| | | Cat. No. 130-113-499 |
| GM-CSF | APC | Miltenyi |
| | | Cat. No. 130-123-420 |
| CD16 | APC- Vio770 | Miltenyi |
| | | Cat. No. 130-113-390 |

### Cytotoxicity Assay/Killing Assay (IncuCyte)

Cytotoxic killing assay was performed via the live-cell imaging system IncuCyte S3, allowing collection of real-time data regarding NK activity. Tumor target cells were labeled with CFSE dye (Life Technologies) and co-cultured with NK cells for 20 hours in a presence of PI (propidium iodide, Sigma) in the media. Viable cells remained unstained whereas dead cells were detected by overlap of the CFSE fluorescence staining and PI.

### EXEMPLARY EMBODIMENTS

Embodiment 1. A method of ex vivo producing natural killer (NK) cells expressing a chimeric antigen receptor (CAR) or a transgenic T cell receptor (tg-TCR) capable of binding HER2, the method comprising:
(a) expanding a population of NK cells by a method comprising:
   (i) culturing said population of NK cells under conditions allowing for cell proliferation, wherein said conditions comprise providing an effective amount of nutrients, serum, IL-15 and nicotinamide; and
   (ii) supplementing said population of NK cells with an effective amount of fresh nutrients, serum, IL-15 and nicotinamide 5-10 days following step (i) to produce expanded NK cells;
   so as to obtain an ex vivo expanded population of NK cells, and
(b) upregulating expression of a CAR or a tg-TCR capable of binding HER2 in said ex vivo expanded population of NK cells, thereby producing the NK cells expressing the CAR or the tg-TCR capable of binding the HER2.

Embodiment 2. The method of embodiment 1, wherein said population of NK cells is derived from cord blood, peripheral blood, bone marrow, CD34+ cells or iPSCs.

Embodiment 3. The method of any one of embodiments 1-2, wherein said population of NK cells is deprived of CD3⁺ cells.

Embodiment 4. The method of any one of embodiments 1-3, wherein said population of NK cells comprises CD3⁻CD56⁺ cells.

Embodiment 5. The method of any one of embodiments 1-4, wherein said effective amount of said nicotinamide comprises an amount between 1.0 mM to 10 mM.

Embodiment 6. The method of any one of embodiments 1-5, wherein said expanding said population of NK cells is affected in the presence of feeder cells or a feeder layer.

Embodiment 7. The method of embodiment 6, wherein said feeder cells comprise irradiated cells.

Embodiment 8. The method of embodiment 6 or 7, wherein said feeder cells comprise T cells or PBMCs.

Embodiment 9. The method of embodiment 8, further comprising a CD3 agonist.

Embodiment 10. The method of any one of embodiments 1-9, wherein said expanding said population of NK cells is affected for 14-16 days.

Embodiment 11. The method of any one of embodiments 1-10, wherein said upregulating expression of said CAR or said tg-TCR is affected on day 12-14 from initiation of culture.

Embodiment 12. The method of any one of embodiments 1-11, wherein said upregulating expression of said CAR or said tg-TCR is affected by mRNA electroporation.

Embodiment 13. The method of any one of embodiments 1-12, wherein said CAR or said tg-TCR is transiently expressed.

Embodiments 14. The method of any one of embodiments 1-13, wherein said CAR comprises at least one co-stimulatory domain.

Embodiment 15. The method of embodiment 14, wherein said at least one co-stimulatory domain is selected from the group consisting of CD28, 2B4, CD137/4-1BB, CD134/OX40, Lsk, ICOS and DAP10.

Embodiment 16. The method of any one of embodiments 1-15, wherein said CAR comprises at least one activating domain.

Embodiment 17. The method of embodiment 16, wherein said activating domain comprises a CD3ζ or FcR-γ.

Embodiment 18. The method of any one of embodiments 1-17, wherein said CAR comprises at least one of a transmembrane domain and a hinge domain.

Embodiment 19. The method of embodiment 18, wherein said transmembrane domain is selected from a CD8, a CD28 and a NKG2D.

Embodiment 20. The method of embodiment 18 or 19, wherein said hinge domain is selected from a CD8 and a CD28.

Embodiment 21. The method of any one of embodiments 1-20, wherein said CAR comprises an antigen binding domain being an antibody or an antigen-binding fragment.

Embodiment 22. The method of embodiment 21, wherein the antigen-binding fragment is a Fab or a scFv.

Embodiment 23. An isolated population of NK cells obtainable according to the method of any one of embodiments 1-22.

Embodiment 24. A pharmaceutical composition comprising the isolated population of NK cells of embodiment 23 and a pharmaceutically active carrier.

Embodiment 25. A method of treating a disease associated with expression of HER2 in a subject in need thereof, the method comprising administering to the subject a therapeutically effective amount of the isolated population of NK cells of embodiment 23, thereby treating the subject.

Embodiment 26. A therapeutically effective amount of the isolated population of NK cells of embodiment 23 for use in treating a disease associated with expression of HER2 in a subject in need thereof.

Embodiment 27. The method of embodiment 25, or the isolated population of NK cells for use of embodiment 26, wherein the disease is a malignant disease.

Embodiment 28. The method or the isolated population of NK cells for use of embodiment 27, wherein said malignant disease is a solid tumor or tumor metastasis.

Embodiment 29. The method or the isolated population of NK cells for use of embodiment 28, wherein said malignant disease is selected from the group consisting of a breast cancer, a gastric cancer, a gastroesophageal cancer, an oesophageal cancer, an ovarian cancer, an endometrial cancer, a lung cancer, an urothelial cancer and a bladder cancer.

Embodiment 30. The method of any one of embodiment 25 or 27-29, or the isolated population of NK cells for use of embodiment 26-29, wherein the subject is a human subject.

### Example 1—NAM Ex Vivo Expanded Cells Expressing Anti-Her2 CAR

In an attempt to treat solid tumor cancer patients with ERBB2 overexpressing tumors, NK chimeric antigen receptor (CAR) cells were developed based on single-chain variable fragment (scFv) of the widely used humanized monoclonal antibody (mAb) Trastuzumab (Herceptin), as previously discussed by Rosenberg et al. (Mol Ther. (2010) 18(4): 843-851).

Different fragments of the signaling moieties that attached the Her2 scFv were used. These were expressed on NK cell membranes using mRNA electroporation.

### Anti-HER2 CAR Construction

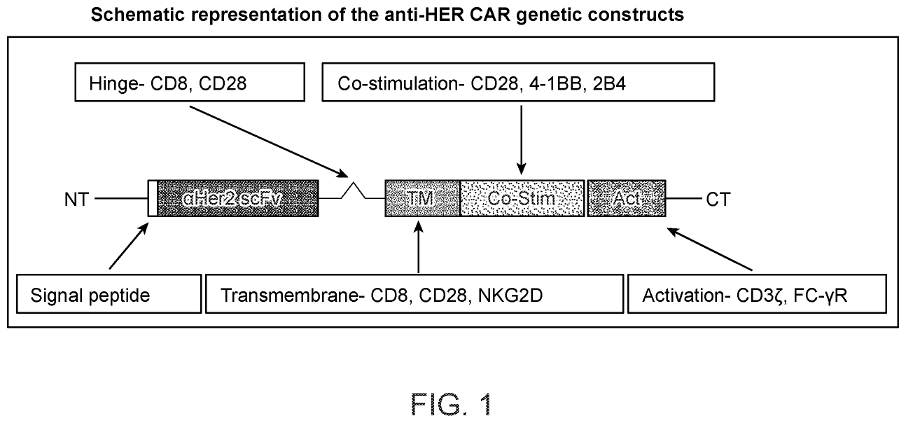

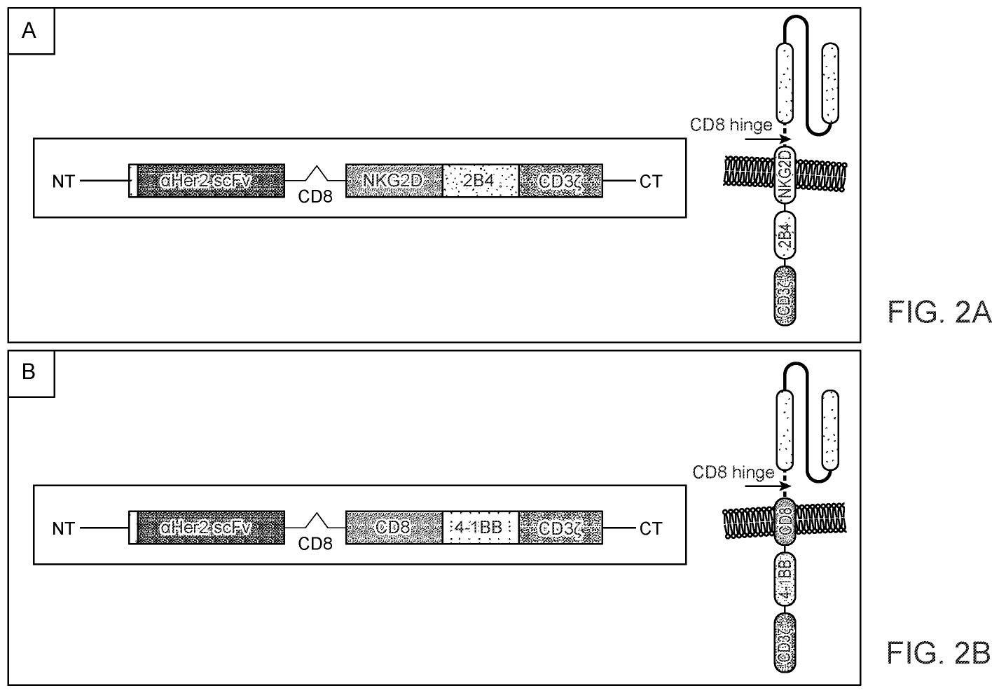

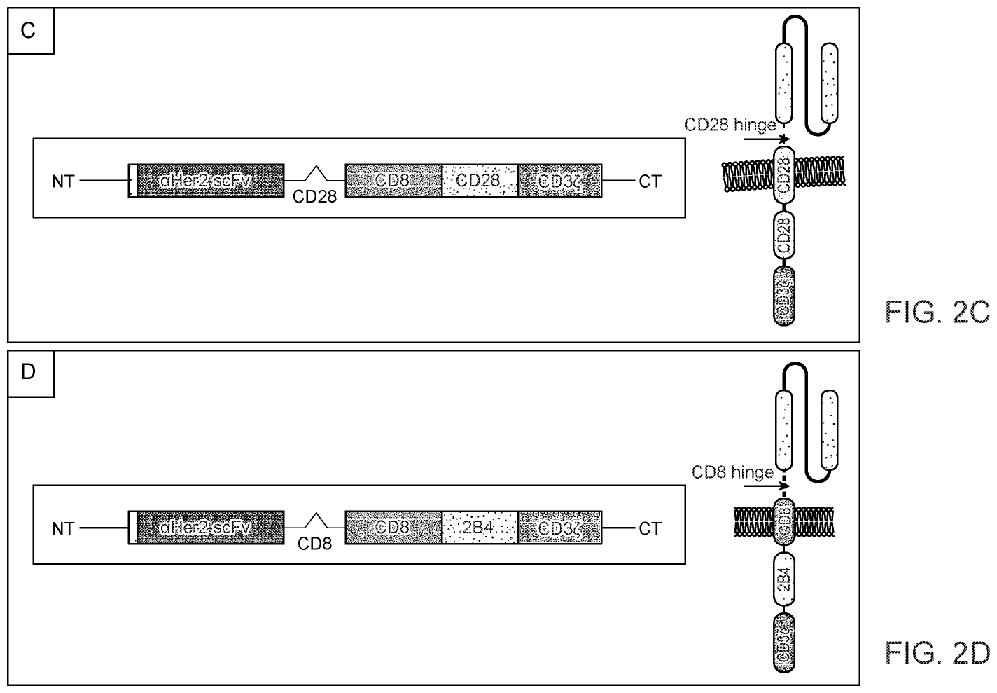

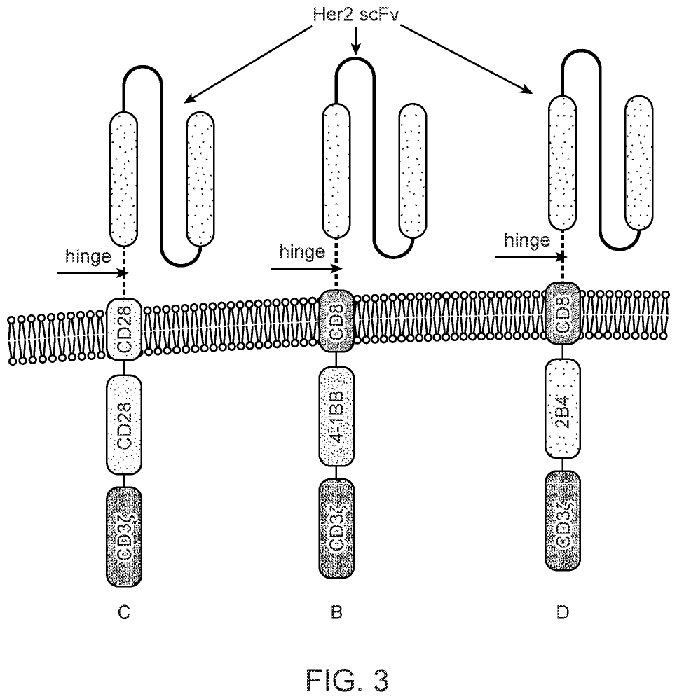

Different constructs were designed for anti-HER2 CAR in a modular way in which the hinge, transmembrane, cytoplasmic domains were modified but the anti-Her2 scFv remained untouched, as depicted in FIGS. 1, 2A-D,3, and 5 and as follows:

### Hinge

The length of the hinge region is important for the formation of the immune synapse. Depending on the antigen distance from the cell surface, the hinge length needs to be adjusted to allow for an optimal distance between the effector and target cell. Amino acid sequences from CD28 or CD8 were used in construction of the anti-HER2 CAR (as specified in SEQ ID Nos: 1-4).

### Transmembrane

The transmembrane (TM) domain consists of a hydrophobic alpha helix that spans the cell membrane and anchors the CAR construct. The choice of TM domain has been shown to affect the functionality of the CAR construct mediated through the degree of cell activation. Amino acid sequences from CD28 or CD8 are most commonly used to date and were used in construction of the anti-HER2 CAR along with the amino acid sequence of NKG2D (as specified in SEQ ID Nos: 5-10).

### Endo-Domains

The evolution of the CAR construct has primarily focused on optimizing the intracellular signaling domains, with the first three generations of CAR constructs referring to the number of activating and co-stimulatory molecules making up the endo-domain. The choice of co-stimulatory domains allows for fine-tuning of the desired NK cell response, whereby CD28-based CARs exhibit an increased cytolytic capacity and shorter persistence compared to 4-1BB-based CARs.

The construction of the anti-HER2 CAR included co-stimulatory domains CD28, 4-1BB and 2B4 with CD3ζ, or FC-γ receptor activating domain (as specified in SEQ ID Nos: 11-18). The full constructs encoding anti-HER2 CAR designated A-D (also called 501.1-501.3 are provided in SEQ ID Nos: 23, 25, 27 and 29, respectively. The full constructs encoding anti-HER2 CAR designated 501.1g-501.4g are provided in SEQ ID Nos: 31, 33, 35 and 37, respectively.

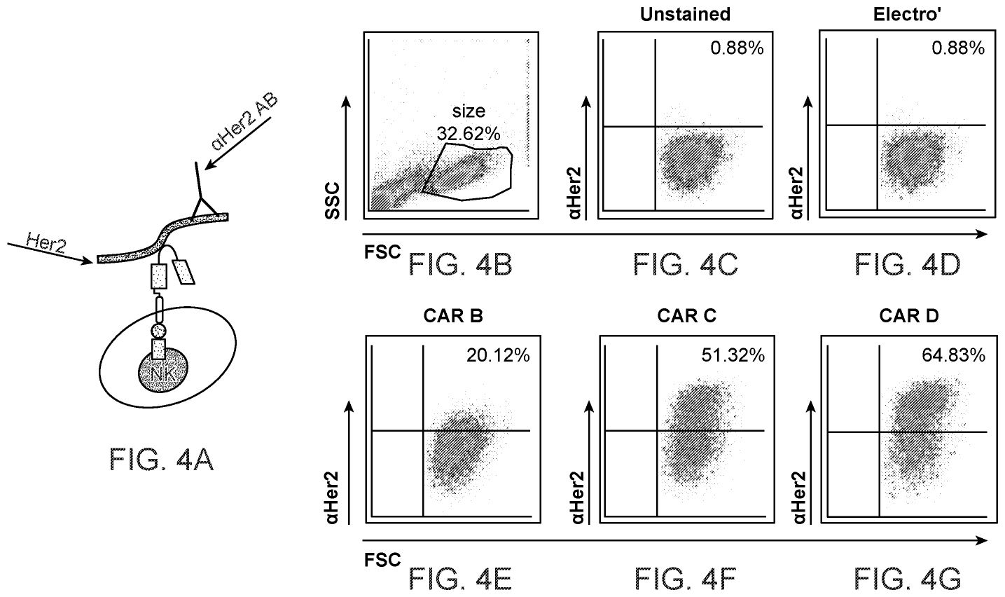

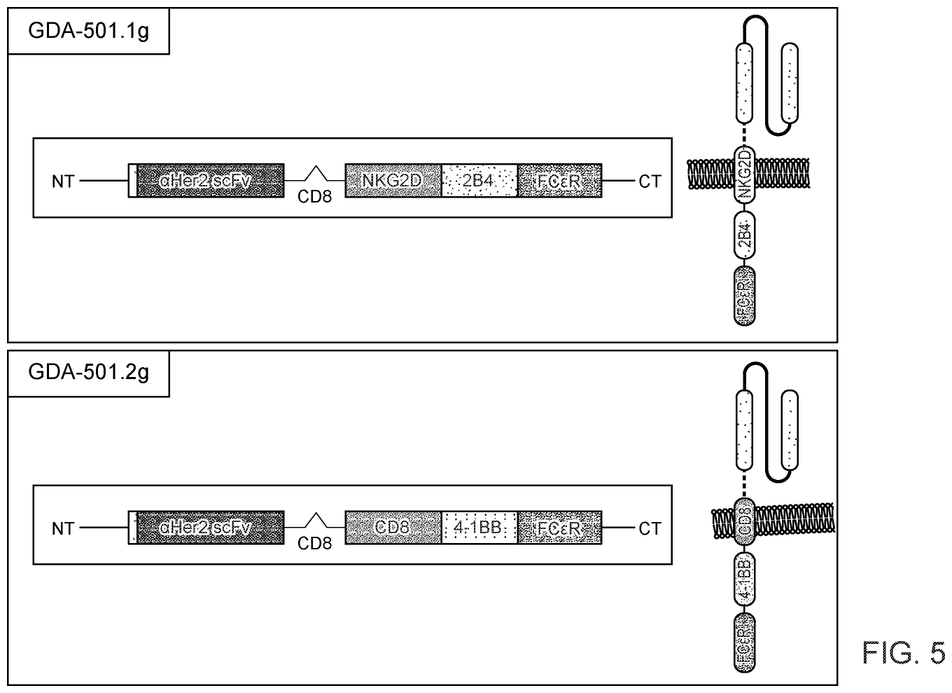

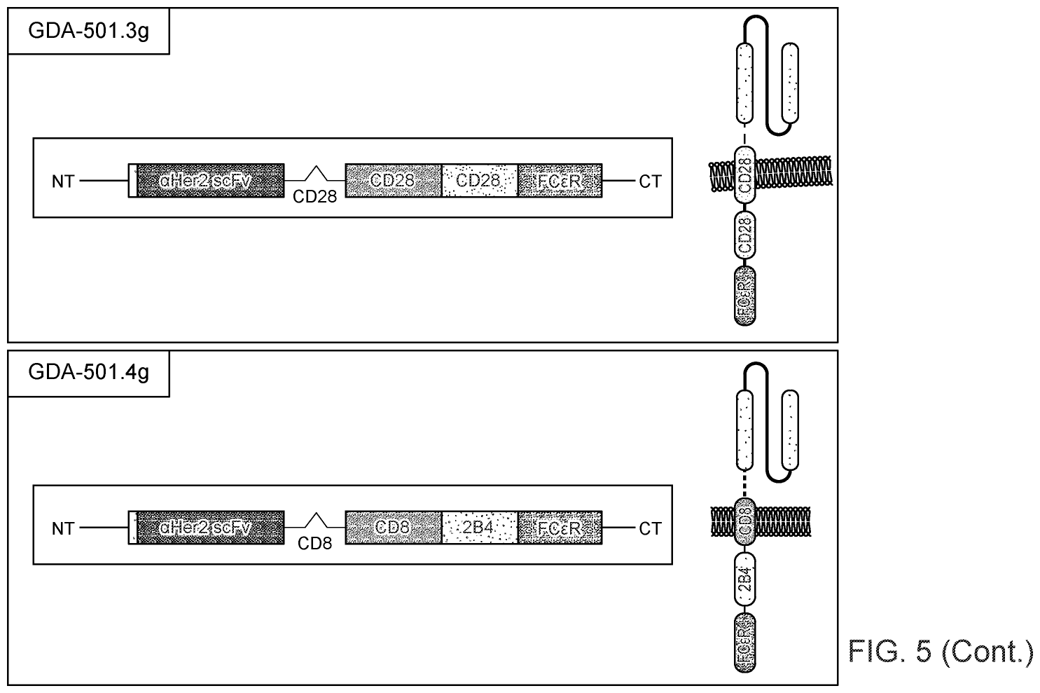

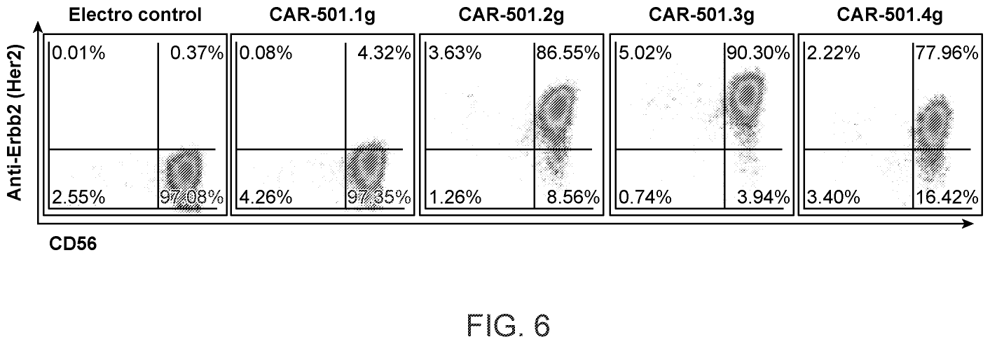

The three CAR constructs designated C, B and D all expressed the anti-HER CAR as evident by the recognition of the HER2 protein (FIGS. 4B-G). Additionally, 501.1g, 501.2g, 501.3g, and 501.4g all expressed the anti-HER CAR (FIG. 6). Using a sandwich flow cytometry technique, the CAR construct expression was identified on NK cells by pre-incubation of NKs with Erbb2 protein followed by anti-Her2 staining.

### Example 2—Potency of 501.1, 501.2, 501.3, 501.4 Anti-HER2 CAR Natural Killer Cells

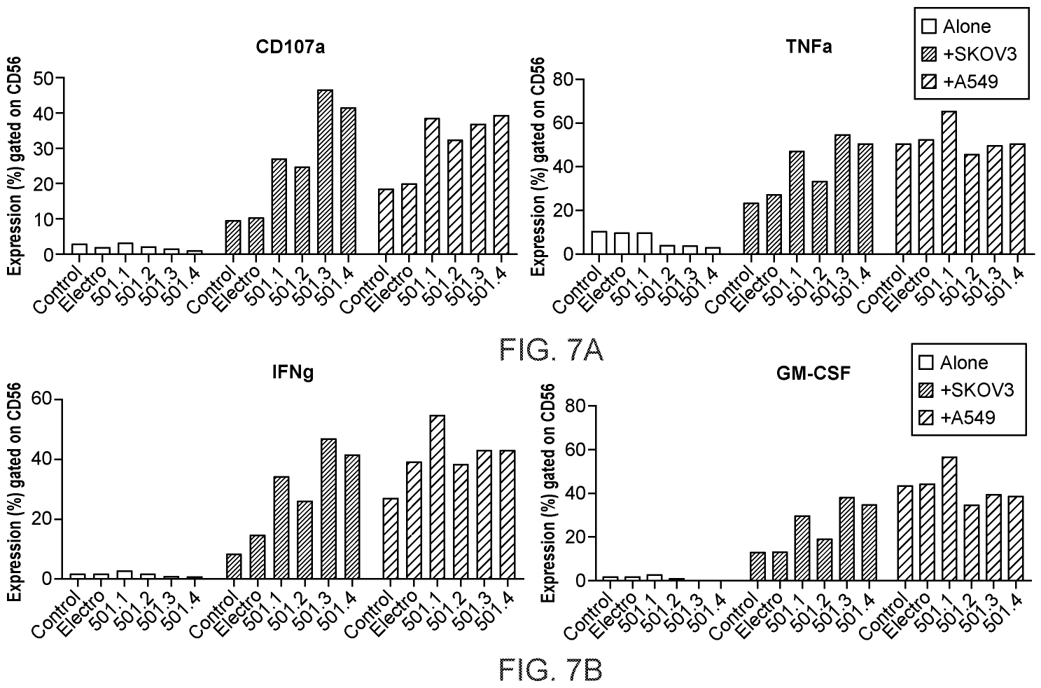

Natural killer cells were electroporated with 30 micrograms of Her-2 CAR expressing mRNA (501.1, 501.2, 501.3, 501.4), respectively. Following the confirmation for CAR expression, cells were co-culture with two Her2 positive cell lines; SKOV3 and A549 for 6 hours. As shown in FIG. 7A-B, the potency analyses showed an increased expression of CD107a, IFNγ, TNFa, and GM-CSF following the expression of each construct.

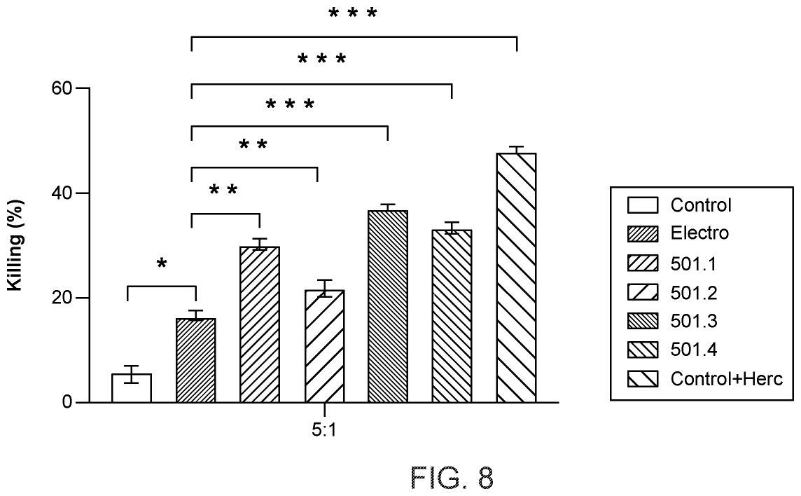

As shown in FIG. 8, significant killing was observed across all Her-2 CAR NKs following a co-culture (in 5:1 effector:target ratio) for 6 hours against SKOV3 cells.

### Example 3—Increased Potency of 501.1g, 501.2g, 501.3g, 501.4g Anti-HER2 CAR Natural Killer Cells

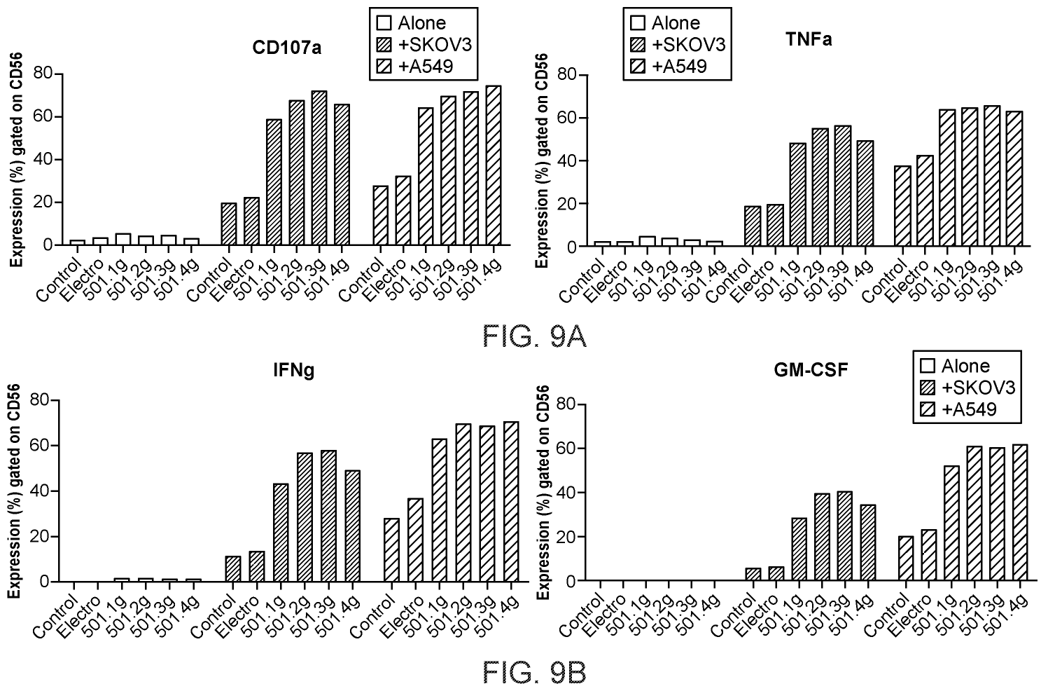

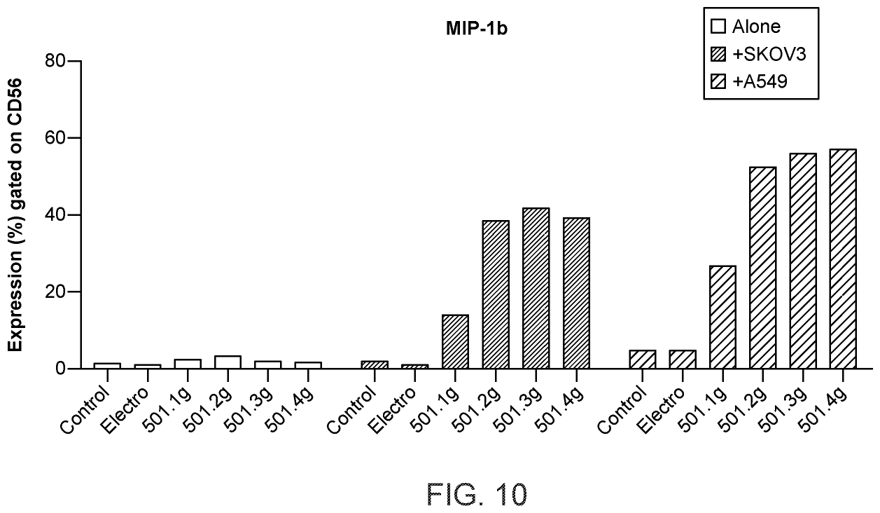

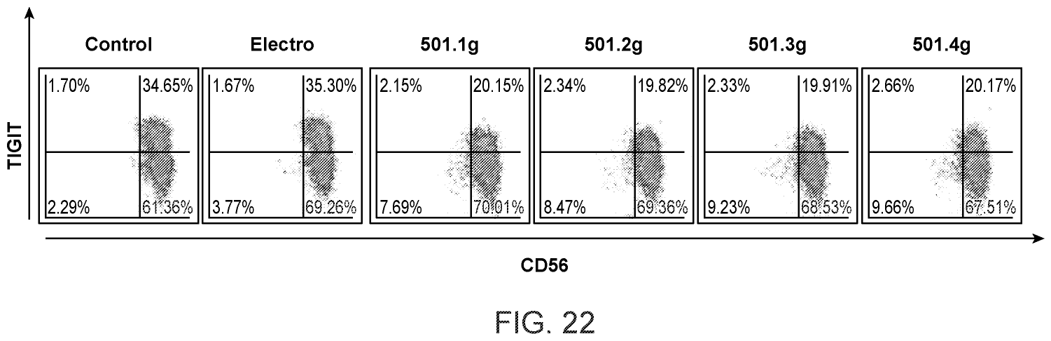

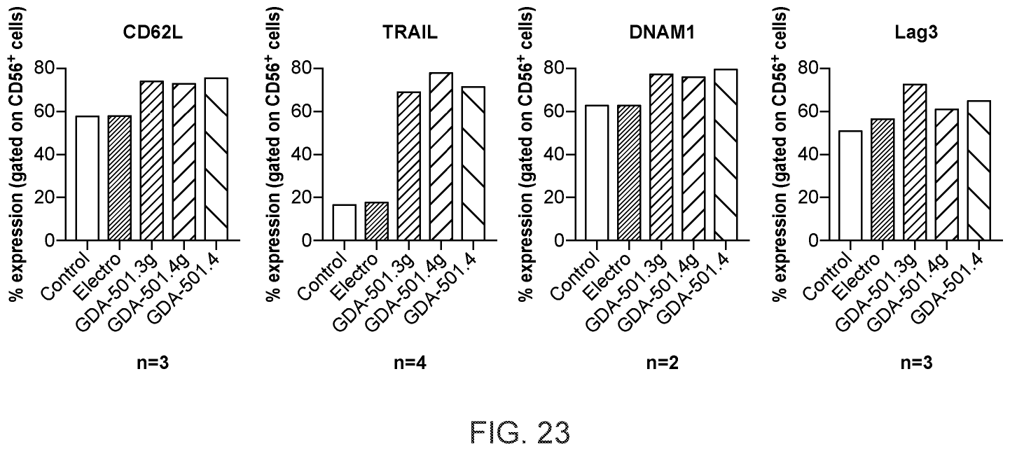

Natural killer cells were electroporated with 20 micrograms of Her-2 CAR expressing mRNA (501.1g, 501.2g, 501.3g, 501.4g), respectively. Following the confirmation of CAR expression, cells were co-culture with two Her2 positive cell lines; SKOV3 and A549 for 6 hours. As shown in FIG. 9A-B and FIG. 10, the potency analyses showed an increased expression of CD107a, IFNγ, TNFa, GM-CSF, and MIP-1b following the expression of each construct. The checkpoint molecule TIGIT demonstrated a slight expression decrease across all CAR-NKs, as compared to control or electroporation alone controls (FIG. 22). Surface marker expression of elevated CD62L, elevated TRAIL, elevated DNAM1, and elevated LAG3 was confirmed in 501.3g, 501.4g, and 501.4, as compared to controls (FIG. 23).

### Example 4—Enhanced Potency and Killing of Her2 Positive Target Cells with Her-2 Natural Killer CARs Expressing 501.1g, 501.2g, 501.3g, 501.4g

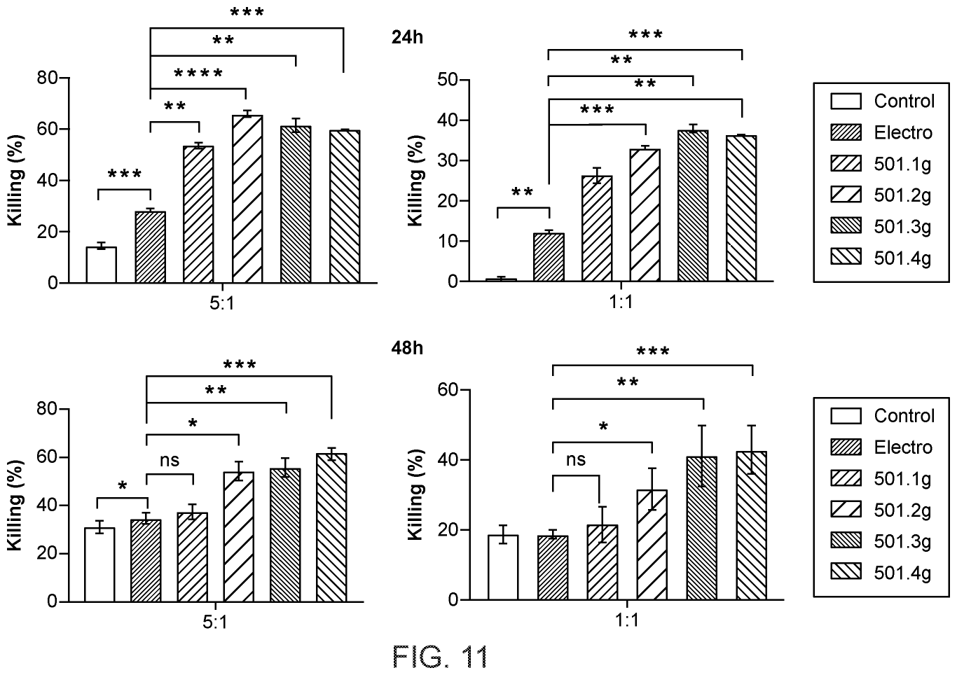

Her-2 natural killer CARs expressing one of 501.1g, 501.2g, 501.3g, 501.4g CAR were generated and co-cultured with Her-2 positive SKOV3 cells either 24 hours or 48 hours post-electroporation of the CAR mRNA to the NK cells. FIG. 11 demonstrates specific-lysis percentages following a 6 hour co-culture (in a NK CAR:SKOV3 ratio of 5:1 or 1:1).

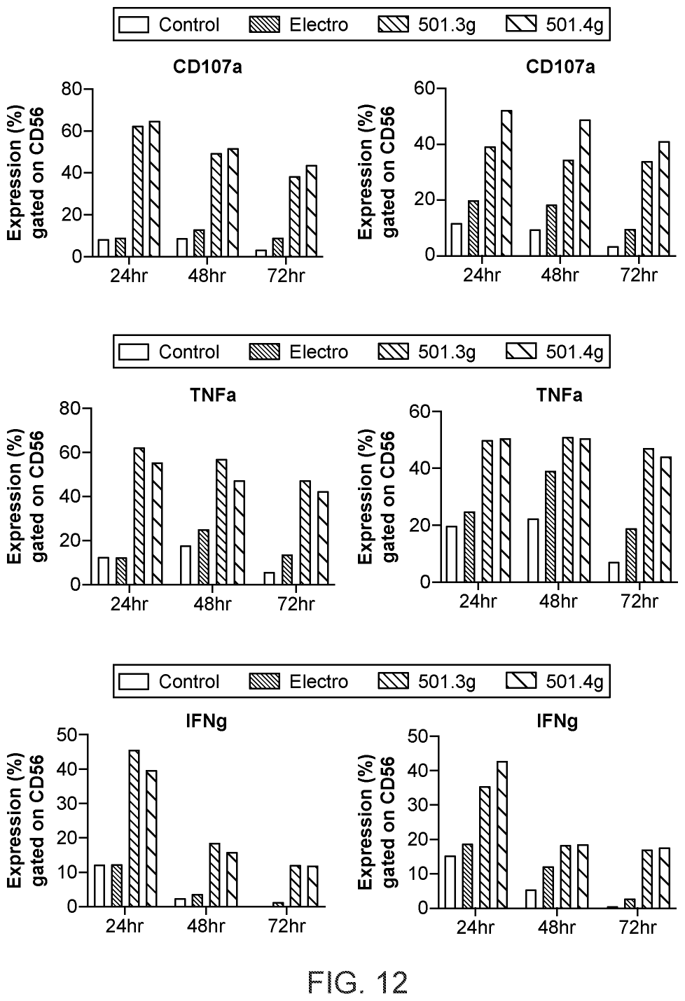

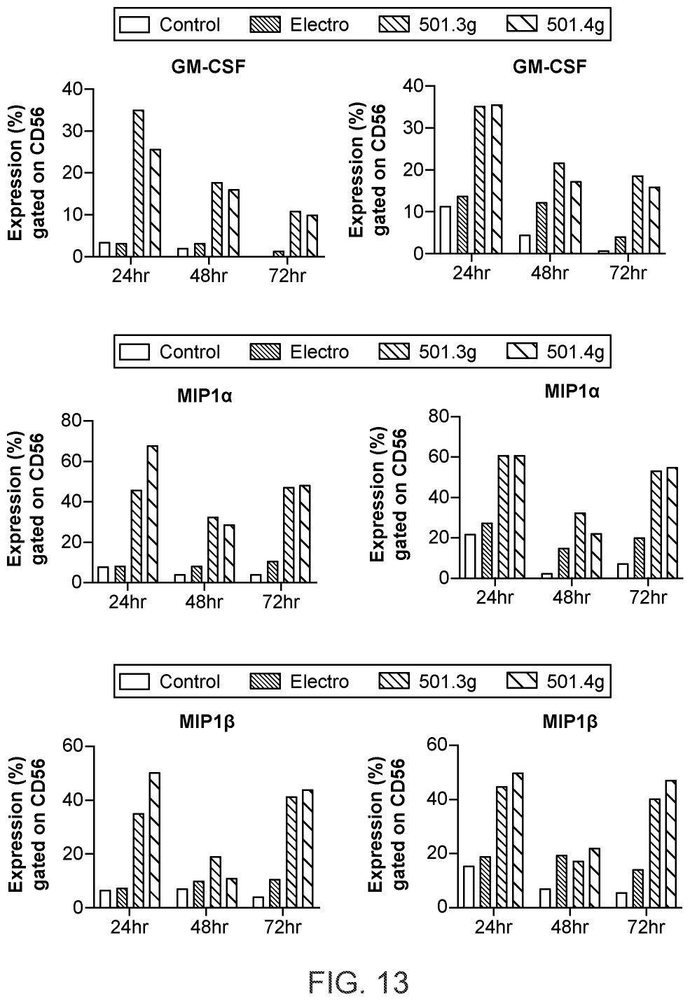

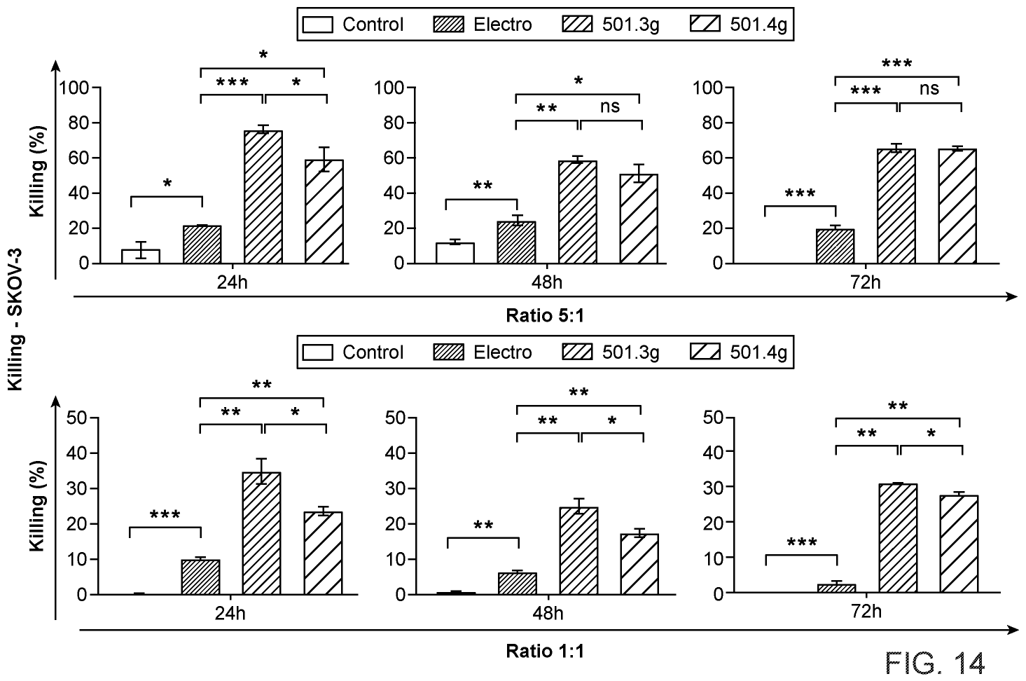

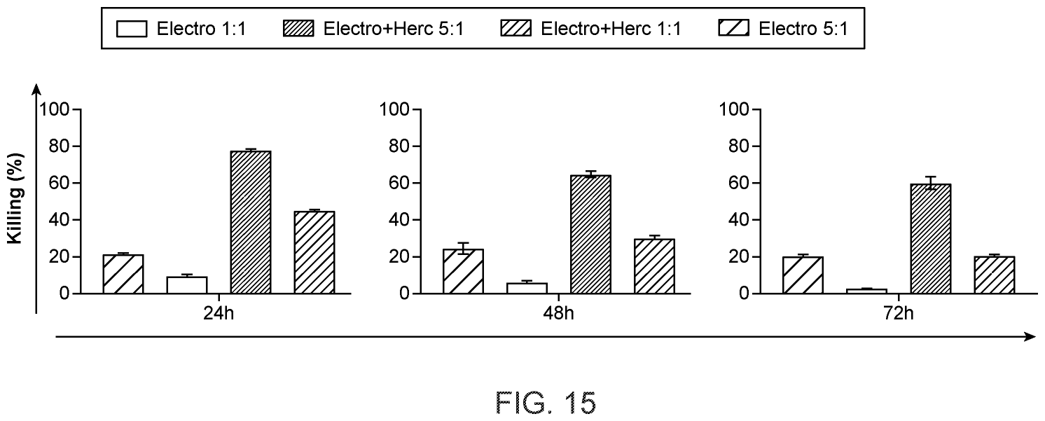

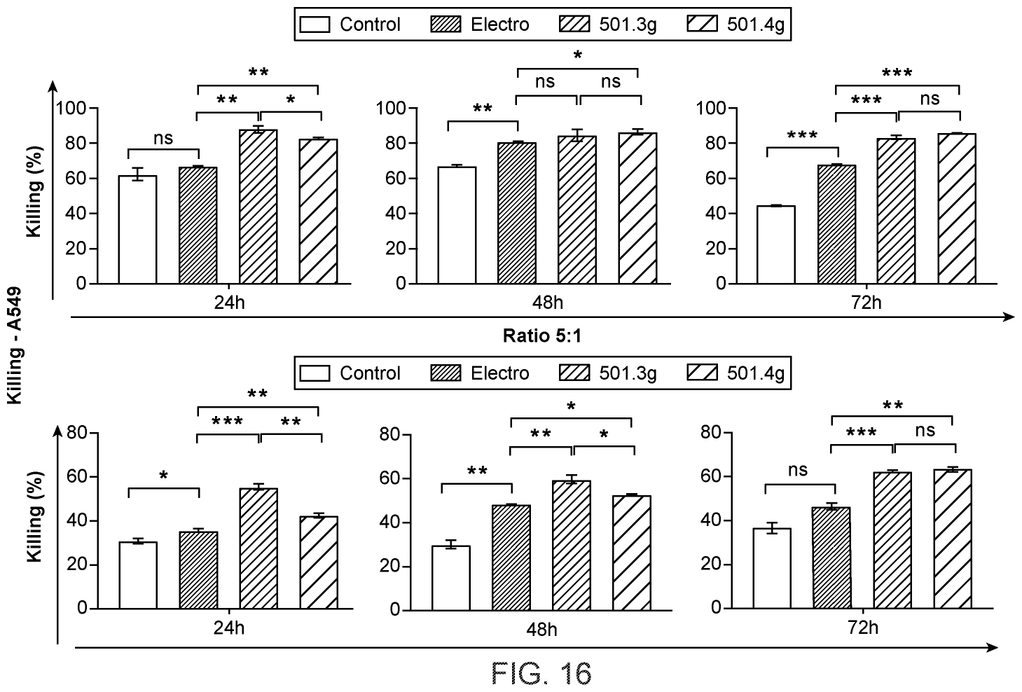

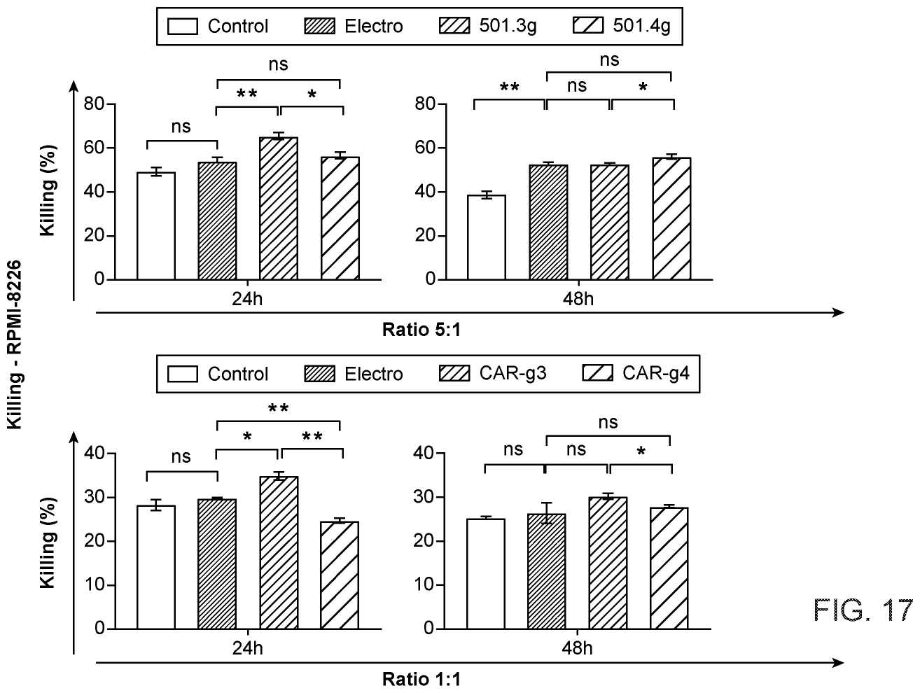

As shown in FIG. 12-13, cytotoxic potency of Her-2 natural killer CARs expressing 501.3g or 501.4g CAR against SKVO3 (FIG. 12-13, left) and A549 (FIG. 12-13, right) is retained after 24-, 48-, and 72-hour post-CAR mRNA electroporation. As shown in FIG. 14, Her-2 natural killer CARs expressing 501.3g or 501.4g CAR demonstrate enhanced killing against on-target SKOV3 cells. The enhanced killing is seen for cells that were co-cultured 24-, 48-, and 72-hour post-CAR (501.3g or 501.4g) mRNA electroporation. As shown in FIG. 15, the killing activity with 501.3g or 501.4g Her2-CAR NK cells was similar as measured with the addition of Herceptin to control (non-CAR expressing) NK cells. FIG. 16 shows that the 501.3g or 501.4g Her2-CAR NK cells also demonstrate enhanced killing, compared to control, of A549 cells across all post-electroporation timepoints tested.

### Example 5—Her-2 Natural Killer CARs Expressing 501.1g, 501.2g, 501.3g, 501.4g Non-Target Activity

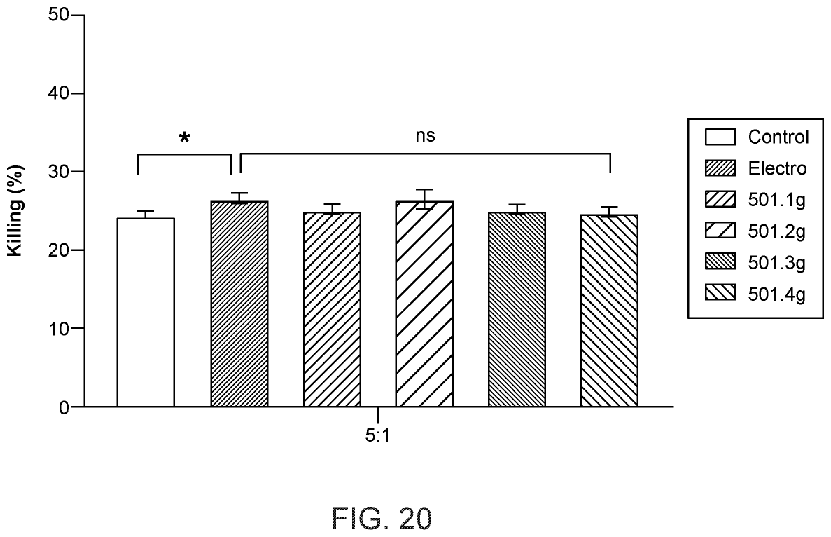

Her-2 natural killer CARs expressing one of 501.1g, 501.2g, 501.3g, 501.4g CAR demonstrated similar killing activity to control NK cells when co-cultured with RPMI-8226 cells, a non-Her-2 expressing cell line. As shown in FIG. 20, insignificant NK CAR killing activity was also observed in non-target Raji cells.

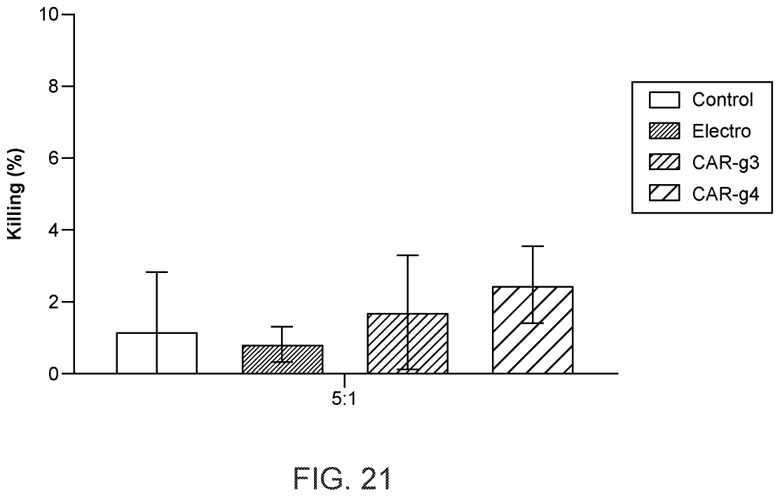

Additionally, as shown in FIG. 20, Her-2 natural killer CARs expressing one of 501.1g, 501.2g, 501.3g, 501.4g CAR demonstrated insignificant killing activity against allogeneic natural killer cells. As shown in FIG. 21, Her-2 natural killer CARs expressing one of 501.1g, 501.2g, 501.3g, 501.4g CAR demonstrated similar killing activity against non-target allogeneic PBMCs.

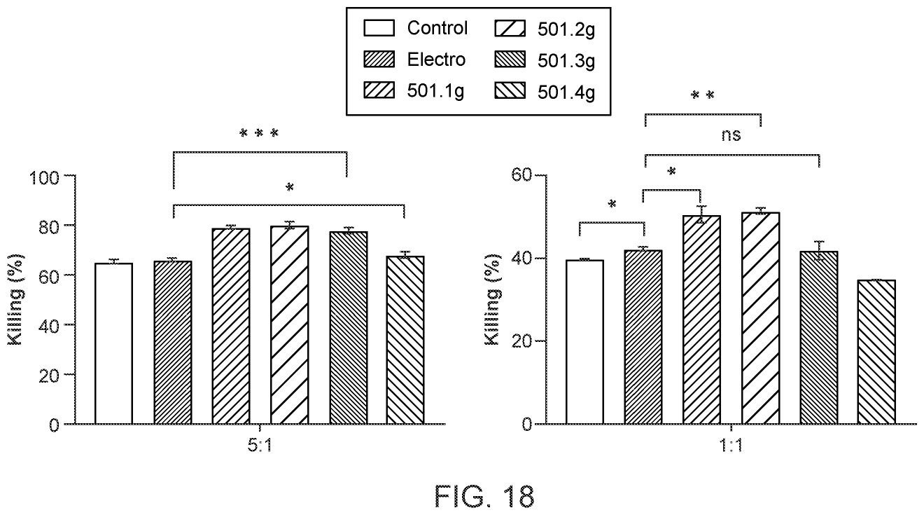

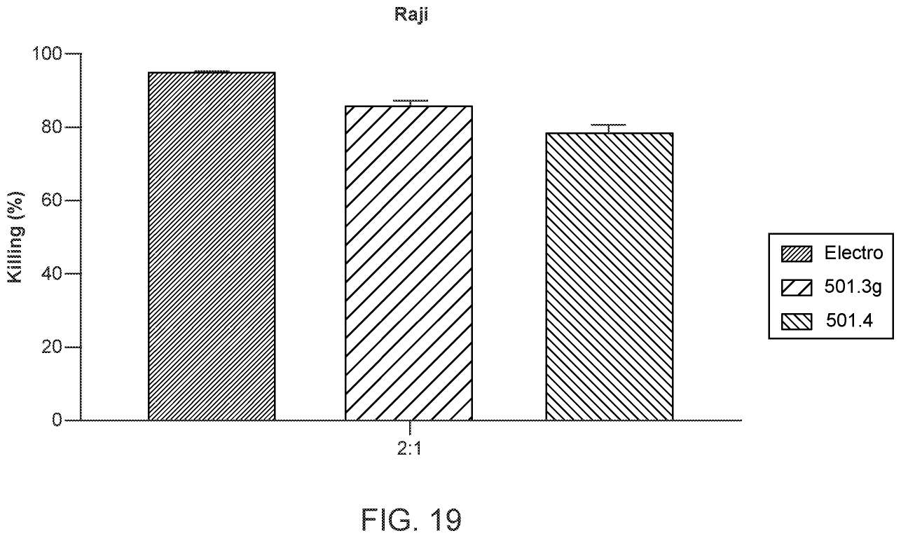

As shown in FIG. 18, 501.3g Her2-CAR NK cells demonstrated insignificant non-target killing of RPMI-8226 cells, across all effector:target ratios tested and post-mRNA CAR electroporation co-culture times.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

SEQUENCE LISTING

| SEQ | | |
|---|---|---|
| ID | | |
| NO | DESCRIPTION | SEQUENCE (5′ to 3′ direction) |
| 1 | CD28 hinge | ATTGAAGTTATGTATCCTCCTCCTTACCTAGACAATGAGAAG |
| | seq: NCBI | AGCAATGGAACCATTATCCATGTGAAAGGGAAACACCTTTGT |
| | Reference | CCAAGTCCCCTATTTCCCGGACCTTCTAAGCCC |
| | Sequence- | |
| | NM_006139.4 | |
| 2 | CD28 hinge aa | IEVMYPPPYLDNEKSNGTIIHVKGKHLCPSPLFPGPSKP |
| | seq | |
| 3 | CD8 hinge | ACCACGACGCCAGCGCCGCGACCACCAACACCGGCGCCCAC |
| | seq: NCBI | CATCGCGTCGCAGCCCCTGTCCCTGCGCCCAGAGGCGTGCC |
| | Reference | GGCCAGCGGGGGGGGCGCAGTGCACACGAGGGGGCTGG |
| | Sequence: | ACTTCGCCTGTGAT |
| | NM_001768.7 | |
| 4 | CD8 hinge aa | TTTPAPRPPTPAPTIASQPLSLRPEACRPAAGGAVHTRGLDFAC |
| | seq | D |
| 5 | CD28 | TTTTGGGTGCTGGTGGTGGTTGGGGGAGTCCTGGCTTGCTA |
| | transmembrane | TAGCTTGCTAGTAACAGTG |
| | seq: NCBI | GCCTTTATTATTTTCTGGGTG |
| | Reference | |
| | Sequence- | |
| | NM_006139.4 | |
| 6 | CD28 | FWVLVVVGGVLACYSLLVTVAFIIFWV |
| | transmembrane | |
| | aa seq | |
| 7 | CD8 | ATCTACATCTGGGCGCCCTTGGCCGGGACTTGTGGGGTCCTT |
| | transmembrane | CTCCTGTCACTGGTTATCACC |
| | seq: NCBI | |
| | Reference | |
| | Sequence: | |
| | NM_001768.7 | |
| 8 | CD8 | IYIWAPLAGTCGVLLLSLVIT |
| | transmembrane | |
| | aa seq | |
| 9 | NKG2D | CCATTTTTTTTCTGCTGCTTCATCGCTGTAGCCATGGGAATCC |
| | transmembrane | GTTTCATTATTATGGTAGCA |
| | seq: NCBI | |
| | Reference | |
| | Sequence: | |
| | NM_007360.4 | |
| 10 | NKG2D | PFFFCCFIAVAMGIRFIIMVA |
| | transmembrane | |
| | aa seq | |
| 11 | CD28 endo | AGGAGTAAGAGGAGCAGGCTCCTGCACAGTGACTACATGAA |
| | domain seq: | CATGACTCCCCGCCGCCCCGGGCCCACCCGCAAGCATTACCA |
| | NCBI | GCCCTATGCCCCACCACGCGACTTCGCAGCCTATCGCTCG |
| | Reference | |
| | Sequence- | |
| | NM_006139.4 | |
| 12 | CD28 endo | RSKRSRLLHSDYMNMTPRRPGPTRKHYQPYAPPRDFAAYRS |
| | domain aa | |
| | seq | |
| 13 | 4-1BB endo | AAACGGGGCAGAAAGAAACTCCTGTATATATTCAAACAACC |
| | domain seq: | ATTTATGAGACCAGTACAAACTACTCAAGAGGAAGATGGCT |
| | NCBI | GTAGCTGCCGATTTCCAGAAGAAGAAGAAGGAGGATGTGA |
| | Reference | ACTG |
| | Sequence: | |
| | NM_001561.6 | |
| 14 | 4-1BB endo | KRGRKKLLYIFKQPFMRPVQTTQEEDGCSCRFPEEEEGGCEL |
| | domain aa | |
| | seq | |
| 15 | 2B4 endo | TGGAGGAGAAAGAGGAAGGAGAAGCAGTCAGAGACCAGTC |
| | domain seq: | CCAAGGAATTTTTGACAATTTACGAAGATGTCAAGGATCTGA |
| | NCBI | AAACCAGGAGAAATCACGAGCAGGAGCAGACTTTTCCTGGA |
| | Reference | GGGGGGAGCACCATCTACTCTATGATCCAGTCCCAGTCTTCT |
| | Sequence: | GCTCCCACGTCACAAGAACCTGCATATACATTATATTCATTAA |
| | NM_016382.4 | TTCAGCCTTCCAGGAAGTCTGGATCCAGGAAGAGGAACCAC |
| | | AGCCCTTCCTTCAATAGCACTATCTATGAAGTGATTGGAAAG |
| | | AGTCAACCTAAAGCCCAGAACCCTGCTCGATTGAGCCGCAAA |
| | | GAGCTGGAGAACTTTGATGTTTATTCC |
| 16 | 2B4 endo | WRRKRKEKQSETSPKEFLTIYEDVKDLKTRRNHEQEQTFPGGGS |
| | domain aa | TIYSMIQSQSSAPTSQEPAYTLYSLIQPSRKSGSRKRNHSPSFNST |
| | seq | IYEVIGKSQPKAQNPARLSRKELENFDVYS |
| 17 | CD3zetta | AGAGTGAAGTTCAGCAGGAGCGCAGACGCCCCCGCGTACCA |
| | endo domain | GCAGGGCCAGAACCAGCTCTATAACGAGCTCAATCTAGGAC |
| | seq: NCBI | GAAGAGAGGAGTACGATGTTTTGGACAAGAGACGTGGCCG |
| | Reference | GGACCCTGAGATGGGGGGAAAGCCGCAGAGAAGGAAGAAC |
| | Sequence: | CCTCAGGAAGGCCTGTACAATGAACTGCAGAAAGATAAGAT |
| | NM_198053.3 | GGCGGAGGCCTACAGTGAGATTGGGATGAAAGGCGAGCGC |
| | | CGGAGGGGCAAGGGGCACGATGGCCTTTACCAGGGTCTCA |
| | | GTACAGCCACCAAGGACACCTACGACGCCCTTCACATGCAG |
| | | GCCCTGCCCCCTCGC |
| 18 | CD3zetta | RVKFSRSADAPAYQQGQNQLYNELNLGRREEYDVLDKRRGRD |
| | endo domain | PEMGGKPQRRKNPQEGLYNELQKDKMAEAYSEIGMKGERRR |
| | aa seq | GKGHDGLYQGLSTATKDTYDALHMQALPPR |
| 19 | Fc fragment | CAAGTGCGAAAGGCAGCTATAACCAGCTATGAGAAATCAGA |
| | of IgE | TGGTGTTTACACGGGCCTGAGCACCAGGAACCAGGAGACTT |
| | receptor Ig | ACGAGACTCTGAAGCATGAGAAACCACCACAG |
| | (FCER1G): | |
| | NCBI | |
| | Reference | |
| | Sequence: | |
| | NM_004106.2 | |
| 20 | Fc fragment | QVRKAAITSYEKSDGVYTGLSTRNQETYETLKHEKPPQ |
| | of IgE | |
| | receptor Ig | |
| | aa seq | |
| 21 | Leader | ATGGATTTTCAGGTGCAGATTTTCAGCTTCCTGCTAATCAGT |
| | Peptide seq: | GCCTCAGTCATAATGTCC |
| | GenBank: | AGAGGA |
| | KF419288.1 | |
| 22 | Leader | MDFQVQIFSFLLISASVIMSRG |
| | Peptide aa | |
| | seq | |
| 23 | GDA-501.A: | ATG GAT TTT CAG GTA CAA ATT TTC TCA TTC CTT CTC ATT |
| | anti-Her2 | TCA GCA TCC GTA ATA ATG TCC AGA GGC GAC ATC CAA |
| | scFv- | ATG ACT CAA TCA CCA AGT TCC TTG TCC GCG TCC GTT |
| | CD8hinge- | GGG GAT AGA GTA ACC ATA ACC TGT AGG GCA TCA CAA |
| | NKG2D TM- | GAT GTG AAT ACG GCC GTA GCG TGG TAT CAA CAG AAA |
| | 2B4-CD3zetta | CCA GGA AAG GCT CCA AAA CTC CTT ATT TAT TCC GCG |
| | Full Seq | AGT TTT CTT TAC AGC GGA GTC CCG AGT AGA TTC TCT |
| | codon | GGT TCT CGC TCT GGC ACT GAT TTT ACT CTG ACC ATA |
| | optimization | AGT TCA CTG CAA CCT GAA GAC TTT GCG ACC TAT TAC |
| | | TGT CAA CAG CAC TAC ACA ACG CCT CCC ACT TTT GGA |
| | | CAA GGT ACT AAA GTA GAG ATT AAA CGG ACC GGA TCC |
| | | ACA AGT GGG AGC GGG AAG CCT GGT TCT GGC GAA GGA |
| | | TCC GAA GTT CAG CTG GTT GAA TCC GGC GGT GGG TTG |
| | | GTG CAA CCC GGG GGG AGC CTG CGC CTC AGC TGT GCC |
| | | GCG AGT GGG TTT AAC ATA AAA GAT ACC TAC ATT CAC |
| | | TGG GTG CGC CAA GCT CCG GGC AAA GGA CTT GAA TGG |
| | | GTC GCG AGG ATC TAC CCG ACC AAC GGT TAC ACA AGA |
| | | TAT GCG GAC TCC GTA AAA GGG CGA TTC ACG ATA TCC |
| | | GCT GAC ACA TCC AAG AAC ACG GCG TAC TTG CAA ATG |
| | | AAT TCT CTT AGG GCC GAG GAC ACC GCA GTT TAT TAC |
| | | TGT AGT CGC TGG GGA GGT GAT GGA TTT TAT GCG ATG |
| | | GAC GTT TGG GGG CAA GGG ACG CTG GTC ACG GTT TCC |
| | | AGT GCT GCA ACC ACA ACG CCA GCA CCA AGA CCT CCC |
| | | ACA CCC GCT CCT ACC ATC GCT TCA CAA CCC CTT TCT |
| | | CTG CGA CCA GAG GCG TGT AGA CCC GCC GCT GGG GGC |
| | | GCC GTT CAC ACG AGG GGC CTG GAC TTC GCG TGC GAC |
| | | CCC TTT TTC TTC TGC TGC TTT ATA GCT GTG GCG ATG |
| | | GGA ATT CGA TTT ATA ATT ATG GTG GCA TGG AGA CGG |
| | | AAG CGG AAG GAG AAA CAG TCC GAG ACT AGC CCG AAG |
| | | GAG TTC TTG ACC ATT TAT GAA GAC GTA AAA GAT TTG |
| | | AAG ACC CGG CGC AAT CAC GAA CAA GAA CAA ACG TTT |
| | | CCA GGA GGC GGT AGT ACT ATA TAC TCA ATG ATT CAA |
| | | AGT CAA TCT TCA GCA CCG ACT TCT CAA GAA CCC GCA |
| | | TAT ACT CTC TAT AGC CTG ATT CAA CCC TCA CGG AAG |
| | | TCA GGG AGC AGG AAA AGG AAC CAT TCA CCG AGT TTT |
| | | AAT TCC ACG ATT TAC GAG GTG ATA GGC AAG AGC CAG |
| | | CCT AAG GCC CAG AAC CCG GCA AGA TTG TCC CGA AAG |
| | | GAA CTC GAA AAC TTT GAT GTG TAC TCT AGG GTG AAA |
| | | TTC AGT CGG TCA GCA GAT GCC CCT GCA TAT CAA CAA |
| | | GGA CAA AAC CAA CTG TAT AAC GAG CTC AAT CTT GGT |
| | | CGA CGC GAG GAA TAC GAC GTA CTT GAC AAG AGA AGA |
| | | GGA AGA GAC CCC GAG ATG GGG GGC AAG CCG CAG |
| | | CGA AGA AAG AAT CCA CAA GAA GGG CTC TAC AAT GAA |
| | | CTG CAG AAG GAC AAA ATG GCC GAA GCC TAT TCC GAG |
| | | ATC GGA ATG AAG GGT GAG CGA AGG CGA GGA AAG |
| | | GGG CAC GAC GGC CTT TAC CAG GGT CTG TCA ACC GCT |
| | | ACG AAA GAC ACG TAC GAC GCC CTG CAC ATG CAA GCG |
| | | CTC CCA CCA CGA TAG |
| 24 | GDA-501.A: | MDFQVQIFSFLLISASVIMSRGDIQMTQSPSSLSASVGDRVTITC |
| | anti-Her2 | RASQDVNTAVAWYQQKPGKAPKLLIYSASFLYSGVPSRFSGSRS |
| | scFv- | GTDFTLTISSLQPEDFATYYCQQHYTTPPTFGQGTKVEIKRTGST |
| | CD8hinge- | SGSGKPGSGEGSEVQLVESGGGLVQPGGSLRLSCAASGFNIKD |
| | NKG2D TM- | TYIHWVRQAPGKGLEWVARIYPTNGYTRYADSVKGRFTISADT |
| | 2B4-CD3zetta | SKNTAYLQMNSLRAEDTAVYYCSRWGGDGFYAMDVWGQGT |
| | Full AA Seq | LVTVSSAATTTPAPRPPTPAPTIASQPLSLRPEACRPAAGGAVHT |
| | | RGLDFACDPFFFCCFIAVAMGIRFIIMVAWRRKRKEKQSETSPK |
| | | EFLTIYEDVKDLKTRRNHEQEQTFPGGGSTIYSMIQSQSSAPTS |
| | | QEPAYTLYSLIQPSRKSGSRKRNHSPSFNSTIYEVIGKSQPKAQN |
| | | PARLSRKELENFDVYSRVKFSRSADAPAYQQGQNQLYNELNLG |
| | | RREEYDVLDKRRGRDPEMGGKPQRRKNPQEGLYNELQKDKM |
| | | AEAYSEIGMKGERRRGKGHDGLYQGLSTATKDTYDALHMQAL |
| | | PPR |
| 25 | GDA-501.B: | ATG GAC TTC CAA GTG CAG ATA TTC TCC TTT CTT CTT |
| | anti-Her2 | ATT TCC GCA TCC GTT ATA ATG AGC AGG GGG GAT ATA |
| | scFv- | CAA ATG ACT CAG TCT CCG TCC TCT CTG AGT GCA TCC |
| | CD8hinge+TM- | GTG GGC GAC CGA GTA ACT ATC ACG TGC CGG GCA TCC |
| | 4-1BB- | CAA GAT GTA AAC ACA GCC GTA GCT TGG TAC CAA CAA |
| | CD3zetta Full | AAA CCA GGT AAA GCT CCT AAA TTG CTT ATT TAC TCT |
| | Seq codon | GCA AGT TTC TTG TAC AGT GGG GTG CCG TCC CGG TTT |
| | optimization | AGT GGC TCC AGA AGC GGC ACT GAT TTC ACT CTG ACA |
| | | ATA TCT TCT CTT CAA CCC GAG GAT TTT GCT ACA TAT |
| | | TAT TGC CAA CAA CAT TAT ACG ACT CCC CCT ACT TTC |
| | | GGC CAA GGG ACT AAA GTC GAG ATC AAA CGA ACC GGT |
| | | TCC ACG TCC GGG TCA GGT AAA CCT GGT TCA GGA GAA |
| | | GGA TCA GAG GTG CAA TTG GTC GAG AGT GGC GGG GGA |
| | | TTG GTA CAA CCA GGT GGC AGT CTT AGG CTG TCT TGT |
| | | GCA GCC TCA GGC TTT AAC ATT AAG GAC ACG TAC ATA |
| | | CAC TGG GTC CGA CAA GCA CCG GGC AAG GGT CTG GAA |
| | | TGG GTG GCA CGG ATT TAT CCT ACA AAC GGA TAT ACT |
| | | CGA TAC GCG GAT TCA GTC AAG GGG CGC TTT ACC ATA |
| | | AGT GCC GAC ACT AGC AAG AAC ACT GCC TAT CTT CAG |
| | | ATG AAT TCA TTG AGG GCG GAA GAC ACT GCG GTA TAC |
| | | TAT TGC TCT AGA TGG GGA GGA GAC GGC TTT TAT GCA |
| | | ATG GAT GTG TGG GGT CAG GGA ACC CTC GTT ACC GTA |
| | | TCT TCT GCT GCA ACC ACC ACC CCC GCC CCC CGA CCA |
| | | CCG ACA CCA GCA CCA ACA ATC GCA TCC CAG CCT TTG |
| | | TCA TTG AGA CCA GAG GCG TGT AGA CCC GCA GCT GGA |
| | | GGC GCA GTC CAT ACG CGG GGG CTG GAT TTT GCC TGC |
| | | GAC ATA TAT ATA TGG GCT CCT CTC GCG GGT ACG TGC |
| | | GGT GTT TTG CTC CTG TCA CTC GTG ATA ACA AAG CGA |
| | | GGC CGG AAA AAA TTG CTC TAT ATC TTC AAA CAG CCG |
| | | TTC ATG CGA CCG GTG CAG ACA ACA CAA GAA GAA GAC |
| | | GGC TGC AGC TGC AGA TTC CCT GAG GAG GAA GAA GGT |
| | | GGG TGT GAA TTG AGG GTT AAA TTC TCC CGA AGC GCG |
| | | GAT GCA CCG GCG TAT CAG CAG GGC CAA AAT CAA CTC |
| | | TAC AAC GAG CTC AAC CTG GGG CGG CGG GAA GAG TAC |
| | | GAC GTA CTG GAC AAG CGC CGA GGC AGA GAC CCT GAG |
| | | ATG GGG GGC AAG CCT CAA AGG CGA AAG AAC CCT CAG |
| | | GAG GGG CTT TAC AAC GAG CTC CAA AAG GAC AAG ATG |
| | | GCG GAG GCC TAT TCA GAA ATC GGC ATG AAA GGC GAA |
| | | CGA AGG AGA GGT AAA GGA CAC GAC GGG CTG TAT CAG |
| | | GGT TTG AGT ACT GCA ACA AAG GAT ACC TAC GAC GCT |
| | | CTC CAC ATG CAA GCG CTG CCC CCA AGG TAG |
| 26 | GDA-501.B: | MDFQVQIFSFLLISASVIMSRGDIQMTQSPSSLSASVGDRVTITC |
| | anti-Her2 | RASQDVNTAVAWYQQKPGKAPKLLIYSASFLYSGVPSRFSGSRS |
| | scFv- | GTDFTLTISSLQPEDFATYYCQQHYTTPPTFGQGTKVEIKRTGST |
| | CD8hinge+TM- | SGSGKPGSGEGSEVQLVESGGGLVQPGGSLRLSCAASGFNIKD |
| | 4-1BB- | TYIHWVRQAPGKGLEWVARIYPTNGYTRYADSVKGRFTISADT |
| | CD3zetta Full | SKNTAYLQMNSLRAEDTAVYYCSRWGGDGFYAMDVWGQGT |
| | AA Seq | LVTVSSAATTTPAPRPPTPAPTIASQPLSLRPEACRPAAGGAVHT |
| | | RGLDFACDIYIWAPLAGTCGVLLLSLVITKRGRKKLLYIFKQPFM |
| | | RPVQTTQEEDGCSCRFPEEEEGGCELRVKFSRSADAPAYQQGQ |
| | | NQLYNELNLGRREEYDVLDKRRGRDPEMGGKPQRRKNPQEGL |
| | | YNELQKDKMAEAYSEIGMKGERRRGKGHDGLYQGLSTATKDT |
| | | YDALHMQALPPR |
| 27 | GDA-501.C: | ATG GAT TTT CAG GTA CAG ATT TTC TCT TTT CTT CTT ATA |
| | anti-Her2 | AGC GCG TCT GTG ATC ATG AGT CGG GGC GAT ATA CAG |
| | scFv- | ATG ACC CAA AGC CCT TCC TCA CTG TCA GCG AGC GTA |
| | CD28hinge- | GGA GAT AGG GTG ACC ATC ACA TGC AGG GCG AGC CAA |
| | TM-Cy- | GAT GTG AAC ACA GCC GTA GCT TGG TAT CAG CAA AAG |
| | CD3zetta Full | CCA GGC AAG GCT CCC AAA CTG CTG ATA TAT TCT GCT |
| | Seq codon | AGC TTT CTG TAT TCC GGT GTA CCC AGC AGG TTC AGC |
| | optimization | GGC TCC AGA AGT GGA ACC GAC TTC ACT CTG ACA ATC |
| | | AGT AGC CTT CAA CCA GAA GAT TTC GCA ACG TAC TAC |
| | | TGT CAA CAA CAT TAC ACG ACG CCC CCA ACC TTC GGG |
| | | CAA GGA ACG AAA GTT GAG ATA AAA AGG ACC GGC AGC |
| | | ACC TCC GGA AGC GGG AAG CCA GGA TCC GGG GAG GGT |
| | | TCC GAG GTG CAA CTC GTC GAG TCA GGT GGC GGT CTC |
| | | GTG CAA CCA GGA GGC TCC CTG CGG CTC TCT TGT GCG |
| | | GCT AGT GGA TTT AAT ATA AAA GAT ACC TAT ATT CAC |
| | | TGG GTG CGC CAA GCA CCT GGA AAA GGG CTG GAG TGG |
| | | GTC GCC AGG ATA TAT CCG ACA AAT GGA TAC ACA CGG |
| | | TAT GCG GAC AGT GTT AAA GGC AGG TTC ACA ATT AGC |
| | | GCA GAC ACG AGC AAG AAT ACA GCC TAT CTT CAG ATG |
| | | AAT TCT CTC AGG GCT GAA GAT ACT GCA GTC TAC TAT |
| | | TGC TCT AGG TGG GGT GGT GAC GGC TTT TAC GCT ATG |
| | | GAT GTC TGG GGG CAG GGC ACT CTG GTT ACT GTC AGC |
| | | TCT GCG ATA GAA GTC ATG TAC CCT CCG CCG TAT CTT |
| | | GAC AAT GAG AAG TCT AAT GGG ACA ATC ATA CAC GTG |
| | | AAA GGC AAG CAC TTG TGC CCC TCT CCC CTG TTC CCC |
| | | GGC CCT AGT AAA CCG TTC TGG GTG CTC GTA GTG GTC |
| | | GGT GGA GTT CTT GCC TGT TAT AGT TTG TTG GTA ACC |
| | | GTC GCG TTT ATA ATA TTC TGG GTC CGG TCC AAG AGA |
| | | AGC CGC CTC CTG CAT TCC GAT TAC ATG AAC ATG ACC |
| | | CCA CGG AGG CCC GGC CCT ACA CGG AAG CAT TAC CAG |
| | | CCA TAC GCT CCG CCT CGA GAT TTT GCT GCT TAT AGG |
| | | TCA CGA GTA AAG TTT AGT AGA TCC GCT GAC GCC CCT |
| | | GCC TAC CAG CAG GGT CAG AAT CAG CTC TAC AAT GAA |
| | | CTG AAC TTG GGC AGG CGA GAA GAG TAT GAC GTT CTC |
| | | GAC AAG CGA AGG GGG AGA GAT CCC GAA ATG GGT |
| | | GGT AAA CCA CAA AGA CGC AAG AAT CCT CAG GAA GGA |
| | | TTG TAC AAC GAG CTC CAA AAG GAT AAG ATG GCG GAA |
| | | GCA TAT TCC GAA ATT GGG ATG AAG GGC GAG CGA AGG |
| | | CGG GGC AAA GGA CAC GAC GGC CTT TAT CAA GGA CTG |
| | | TCT ACG GCT ACT AAA GAC ACT TAT GAT GCG CTG CAC |
| | | ATG CAA GCA TTG CCG CCG AGA TAG |
| 28 | GDA-501.C: | MDFQVQIFSFLLISASVIMSRGDIQMTQSPSSLSASVGDRVTITC |
| | anti-Her2 | RASQDVNTAVAWYQQKPGKAPKLLIYSASFLYSGVPSRFSGSRS |
| | scFv- | GTDFTLTISSLQPEDFATYYCQQHYTTPPTFGQGTKVEIKRTGST |
| | CD28hinge- | SGSGKPGSGEGSEVQLVESGGGLVQPGGSLRLSCAASGFNIKD |
| | TM-Cy- | TYIHWVRQAPGKGLEWVARIYPTNGYTRYADSVKGRFTISADT |
| | CD3zetta Full | SKNTAYLQMNSLRAEDTAVYYCSRWGGDGFYAMDVWGQGT |
| | Seq | LVTVSSAIEVMYPPPYLDNEKSNGTIIHVKGKHLCPSPLFPGPSK |
| | | PFWVLVVVGGVLACYSLLVTVAFIIFWVRSKRSRLLHSDYMNM |
| | | TPRRPGPTRKHYQPYAPPRDFAAYRSRVKFSRSADAPAYQQGQ |
| | | NQLYNELNLGRREEYDVLDKRRGRDPEMGGKPQRRKNPQEGL |
| | | YNELQKDKMAEAYSEIGMKGERRRGKGHDGLYQGLSTATKDT |
| | | YDALHMQALPPR |
| 29 | GDA-501.D: | ATG GAT TTC CAA GTC CAA ATC TTT AGT TTT TTG CTT ATT |
| | anti-Her2 | TCA GCC TCC GTC ATT ATG AGT AGA GGG GAT ATA CAA |
| | scFv- | ATG ACC CAG TCC CCT AGT AGC CTG AGC GCA AGC GTT |
| | CD8hinge+TM- | GGA GAC CGG GTA ACC ATA ACA TGT AGG GCT AGC CAG |
| | 2B4- | GAC GTT AAT ACA GCC GTT GCG TGG TAT CAG CAA AAA |
| | CD3zetta Full | CCG GGC AAA GCA CCT AAA CTC CTG ATC TAT AGC GCC |
| | Seq codon | TCA TTC CTG TAT AGC GGC GTC CCT AGT CGA TTC TCA |
| | optimization | GGA AGT AGA TCT GGT ACT GAC TTC ACG TTG ACC ATA |
| | | TCC TCA CTC CAA CCC GAG GAC TTC GCA ACA TAT TAT |
| | | TGC CAG CAG CAC TAT ACC ACG CCG CCG ACG TTC GGG |
| | | CAA GGG ACC AAA GTC GAA ATT AAG AGA ACA GGG TCT |
| | | ACG AGT GGC AGT GGT AAA CCC GGT TCC GGC GAG GGG |
| | | TCC GAA GTG CAA TTG GTG GAA TCT GGA GGG GGT CTC |
| | | GTA CAA CCG GGG GGC TCC CTT AGA CTC AGT TGT GCC |
| | | GCG AGC GGC TTC AAT ATT AAG GAC ACT TAT ATC CAT |
| | | TGG GTT CGG CAA GCT CCC GGC AAG GGT CTT GAA TGG |
| | | GTA GCA CGC ATA TAC CCG ACA AAT GGA TAC ACG CGG |
| | | TAC GCG GAT AGT GTC AAG GGT AGG TTT ACC ATA TCT |
| | | GCG GAC ACG TCC AAA AAC ACC GCC TAC CTT CAG ATG |
| | | AAT TCC CTC CGC GCT GAA GAC ACT GCG GTG TAT TAC |
| | | TGC AGC CGC TGG GGC GGG GAT GGG TTT TAC GCC ATG |
| | | GAT GTG TGG GGT CAG GGT ACA CTC GTA ACT GTG AGC |
| | | AGC GCC ACC ACG ACG CCC GCA CCG AGG CCA CCG ACT |
| | | CCA GCA CCC ACT ATC GCT TCT CAA CCT CTG TCA CTG |
| | | CGC CCT GAA GCC TGT CGG CCT GCG GCC GGG GGA GCG |
| | | GTT CAT ACG CGG GGG CTG GAT TTC GCT TGC GAC ATT |
| | | TAT ATA TGG GCA CCC CTC GCG GGT ACA TGT GGC GTC |
| | | CTC TTG CTG AGT CTC GTA ATT ACG TGG AGA CGA AAG |
| | | AGG AAA GAG AAG CAG TCT GAA ACT AGC CCT AAG GAG |
| | | TTC CTT ACT ATA TAT GAG GAT GTT AAA GAT CTG AAA |
| | | ACC CGA AGA AAC CAT GAA CAG GAA CAA ACT TTC CCT |
| | | GGC GGG GGA AGT ACG ATT TAC AGC ATG ATC CAA TCT |
| | | CAG TCA AGT GCG CCA ACC AGT CAA GAA CCT GCG TAT |
| | | ACA TTG TAT TCC CTC ATT CAG CCA TCT AGG AAA AGC |
| | | GGT TCA CGC AAA AGG AAC CAT AGC CCT TCT TTC AAT |
| | | TCA ACC ATC TAT GAA GTT ATT GGT AAA AGT CAA CCT |
| | | AAA GCG CAA AAT CCG GCG CGA CTT AGC CGC AAA GAA |
| | | CTC GAA AAC TTC GAT GTA TAC AGT CGG GTA AAA TTC |
| | | TCT CGC AGT GCC GAT GCG CCT GCC TAC CAG CAG GGA |
| | | CAG AAT CAG TTG TAC AAC GAA CTG AAC CTC GGC CGA |
| | | AGA GAG GAG TAT GAT GTT CTT GAT AAG CGG AGG GGC |
| | | AGA GAT CCC GAG ATG GGT GGG AAG CCA CAA AGA CGA |
| | | AAA AAT CCT CAG GAG GGA CTG TAC AAT GAA CTT CAA |
| | | AAA GAC AAG ATG GCT GAA GCC TAC TCT GAG ATT GGG |
| | | ATG AAA GGT GAG CGC CGA AGG GGA AAG GGC CAC |
| | | GAC GGC TTG TAT CAA GGA CTG TCC ACC GCC ACT AAA |
| | | GAT ACG TAC GAT GCC TTG CAT ATG CAA GCC CTT CCT |
| | | CCA CGC TAG |
| 30 | GDA-501.D: | MDFQVQIFSFLLISASVIMSRGDIQMTQSPSSLSASVGDRVTITC |
| | anti-Her2 | RASQDVNTAVAWYQQKPGKAPKLLIYSASFLYSGVPSRFSGSRS |
| | scFv- | GTDFTLTISSLQPEDFATYYCQQHYTTPPTFGQGTKVEIKRTGST |
| | CD8hinge+TM- | SGSGKPGSGEGSEVQLVESGGGLVQPGGSLRLSCAASGFNIKD |
| | 2B4- | TYIHWVRQAPGKGLEWVARIYPTNGYTRYADSVKGRFTISADT |
| | CD3zetta Full | SKNTAYLQMNSLRAEDTAVYYCSRWGGDGFYAMDVWGQGT |
| | AA Seq | LVTVSSATTTPAPRPPTPAPTIASQPLSLRPEACRPAAGGAVHTR |
| | | GLDFACDIYIWAPLAGTCGVLLLSLVITWRRKRKEKQSETSPKEF |
| | | LTIYEDVKDLKTRRNHEQEQTFPGGGSTIYSMIQSQSSAPTSQE |
| | | PAYTLYSLIQPSRKSGSRKRNHSPSFNSTIYEVIGKSQPKAQNPA |
| | | RLSRKELENFDVYSRVKFSRSADAPAYQQGQNQLYNELNLGRR |
| | | EEYDVLDKRRGRDPEMGGKPQRRKNPQEGLYNELQKDKMAE |
| | | AYSEIGMKGERRRGKGHDGLYQGLSTATKDTYDALHMQALPPR |
| 31 | GDA-501.1g: | ATG GAC TTT CAA GTC CAG ATT TTC TCC TTC CTT CTC ATT |
| | anti-Her2 | AGC GCC AGC GTA ATA ATG AGT CGA GGT GAC ATT CAA |
| | scFv- | ATG ACA CAG AGT CCC AGT TCC CTC AGT GCT TCC GTA G |
| | CD8hinge- | GC GAC AGA GTT ACT ATT ACG TGT AGG GCT TCA CAA GA |
| | NKG2D TM- | T GTT AAT ACG GCC GTG GCT TGG TAC CAG CAA AAA CCT |
| | 2B4- | GGA AAG GCT CCA AAA TTG CTT ATA TAC TCA GCT TCA T |
| | FCepsilonR | TC CTC TAC TCC GGG GTC CCG TCA AGG TTC TCT GGT TCC |
| | Full Seq | AGG TCA GGC ACA GAT TTC ACA CTC ACA ATC TCC TCT C |
| | codon | TG CAG CCG GAA GAC TTT GCG ACA TAC TAT TGT CAG CA |
| | optimization | A CAT TAT ACG ACC CCG CCC ACT TTC GGA CAA GGT ACT |
| | | AAG GTT GAA ATC AAG CGA ACG GGT TCA ACA AGC GGC |
| | | TCA GGA AAA CCA GGG TCC GGA GAA GGT TCT GAG GTC |
| | | CAG CTG GTC GAG TCA GGC GGT GGT CTG GTC CAA CCG |
| | | GGC GGC TCA CTT AGA CTG AGC TGC GCA GCG TCT GGG |
| | | TTT AAT ATA AAG GAC ACC TAT ATA CAT TGG GTA AGG C |
| | | AA GCG CCC GGG AAA GGA CTG GAG TGG GTT GCA AGG |
| | | ATT TAC CCA ACG AAT GGT TAT ACA CGC TAT GCT GAT A |
| | | GC GTA AAA GGT CGG TTT ACA ATA TCT GCT GAC ACC AG |
| | | C AAG AAT ACA GCG TAC CTT CAA ATG AAC TCT TTG CGG |
| | | GCC GAG GAT ACT GCT GTT TAT TAC TGC TCT CGC TGG G |
| | | GC GGT GAC GGA TTT TAT GCC ATG GAC GTA TGG GGA C |
| | | AG GGG ACT CTT GTC ACA GTT TCC AGT GCC GCT ACC AC |
| | | T ACC CCC GCG CCA CGA CCA CCA ACT CCA GCA CCC ACT |
| | | ATA GCA TCT CAG CCA CTG TCC CTG AGG CCC GAA GCG T |
| | | GT CGA CCC GCG GCA GGT GGC GCA GTG CAT ACT CGC G |
| | | GA TTG GAT TTC GCT TGT GAT CCC TTT TTC TTT TGT TGC |
| | | TTT ATC GCC GTG GCG ATG GGT ATT CGA TTT ATA ATT AT |
| | | G GTG GCG TGG CGG CGG AAG AGA AAA GAG AAG CAA A |
| | | GC GAA ACT TCT CCA AAG GAG TTC TTG ACT ATA TAT GA |
| | | A GAC GTG AAG GAT CTC AAG ACG CGC CGC AAT CAT GA |
| | | G CAG GAG CAG ACT TTC CCT GGG GGC GGG AGC ACC AT |
| | | A TAT TCA ATG ATC CAA AGT CAG TCT AGC GCA CCC ACC |
| | | TCA CAA GAG CCG GCC TAT ACC CTG TAC TCC CTC ATC CA |
| | | G CCT AGC AGG AAA TCT GGA AGC CGA AAG CGA AAT CA |
| | | T AGT CCG AGT TTC AAC AGT ACG ATA TAT GAA GTA ATA |
| | | GGT AAA TCA CAA CCC AAA GCC CAG AAT CCA GCT CGC C |
| | | TT TCT CGG AAG GAG CTG GAG AAT TTT GAC GTC TAT TC |
| | | C CAA GTG CGG AAA GCC GCA ATT ACC TCC TAC GAA AAA |
| | | AGC GAT GGT GTT TAC ACG GGC TTG AGC ACA CGG AAC |
| | | CAG GAA ACT TAT GAG ACC TTG AAA CAT GAA AAG CCA |
| | | CCG CAA TAG |
| 32 | GDA-501.1g: | MDFQVQIFSFLLISASVIMSRGDIQMTQSPSSLSASVGDRVTITC |
| | anti-Her2 | RASQDVNTAVAWYQQKPGKAPKLLIYSASFLYSGVPSRFSGSRS |
| | scFv- | GTDFTLTISSLQPEDFATYYCQQHYTTPPTFGQGTKVEIKRTGST |
| | CD8hinge- | SGSGKPGSGEGSEVQLVESGGGLVQPGGSLRLSCAASGFNIKD |
| | NKG2D TM- | TYIHWVRQAPGKGLEWVARIYPTNGYTRYADSVKGRFTISADT |
| | 2B4- | SKNTAYLQMNSLRAEDTAVYYCSRWGGDGFYAMDVWGQGT |
| | FCepsilonR | LVTVSSAATTTPAPRPPTPAPTIASQPLSLRPEACRPAAGGAVHT |
| | Full AA Seq | RGLDFACDPFFFCCFIAVAMGIRFIIMVAWRRKRKEKQSETSPK |
| | | EFLTIYEDVKDLKTRRNHEQEQTFPGGGSTIYSMIQSQSSAPTS |
| | | QEPAYTLYSLIQPSRKSGSRKRNHSPSFNSTIYEVIGKSQPKAQN |
| | | PARLSRKELENFDVYSQVRKAAITSYEKSDGVYTGLSTRNQETYE |
| | | TLKHEKPPQ*X |
| 33 | GDA-501.2g: | ATG GAT TTC CAG GTT CAG ATT TTC TCC TTC CTG TTG AT |
| | anti-Her2 | C AGT GCG AGT GTG ATT ATG TCT CGA GGT GAT ATA CAG |
| | scFv- | ATG ACG CAA AGT CCA TCC TCA CTG AGT GCA AGC GTG |
| | CD8hinge+TM- | GGT GAC AGG GTT ACT ATC ACT TGT AGG GCA AGC CAG |
| | 4-1BB- | GAC GTG AAT ACT GCA GTC GCT TGG TAC CAG CAG AAG |
| | FCepsilonR | CCT GGA AAA GCG CCA AAG CTG CTG ATT TAT AGC GCT A |
| | Full Seq | GT TTT CTG TAT AGC GGT GTA CCG TCA CGG TTC TCT GG |
| | codon | G AGC CGG TCT GGA ACG GAT TTT ACC CTT ACC ATT TCT |
| | optimization | TCA CTC CAA CCA GAA GAT TTT GCC ACT TAT TAT TGC CA |
| | | A CAG CAT TAC ACC ACT CCC CCG ACA TTC GGA CAA GGG |
| | | ACG AAA GTA GAG ATC AAA CGC ACG GGC TCC ACA AGT |
| | | GGA AGT GGG AAA CCT GGC TCC GGT GAG GGA TCA GAG |
| | | GTT CAG CTG GTG GAA TCC GGG GGC GGT CTT GTA CAG |
| | | CCG GGT GGA TCA CTT AGG TTG AGC TGC GCT GCA TCA |
| | | GGA TTC AAT ATC AAG GAC ACA TAC ATC CAC TGG GTG C |
| | | GC CAA GCT CCG GGG AAG GGT TTG GAA TGG GTT GCA C |
| | | GC ATT TAT CCC ACG AAT GGC TAC ACT CGG TAT GCT GA |
| | | C TCT GTG AAA GGA CGG TTT ACT ATC TCC GCC GAC ACG |
| | | AGT AAG AAT ACA GCG TAT CTT CAG ATG AAC TCT CTC A |
| | | GG GCA GAG GAC ACA GCA GTG TAC TAT TGC AGC AGG T |
| | | GG GGA GGG GAT GGC TTC TAC GCT ATG GAC GTA TGG G |
| | | GC CAA GGG ACG TTG GTA ACC GTG TCT TCC GCC GCA AC |
| | | T ACC ACG CCA GCG CCT CGA CCA CCC ACT CCC GCA CCC |
| | | ACA ATT GCG AGT CAA CCG CTG TCA CTG AGA CCA GAA |
| | | GCT TGC AGA CCC GCC GCA GGT GGC GCG GTA CAT ACT |
| | | CGC GGG CTG GAC TTC GCC TGT GAT ATC TAT ATC TGG G |
| | | CT CCT TTG GCT GGC ACG TGC GGC GTG CTT CTC CTT TCA |
| | | CTC GTG ATC ACA AAA AGG GGA CGG AAG AAG CTC TTG |
| | | TAT ATT TTC AAA CAG CCG TTT ATG CGC CCG GTA CAG A |
| | | CT ACG CAA GAA GAA GAC GGT TGC TCC TGC AGA TTT CC |
| | | T GAG GAA GAG GAA GGC GGC TGC GAG TTG CAG GTT A |
| | | GG AAA GCC GCG ATA ACT AGC TAC GAG AAG AGT GAT G |
| | | GG GTA TAT ACG GGC CTC TCC ACA AGG AAC CAG GAA A |
| | | CA TAT GAG ACT CTT AAA CAT GAA AAA CCT CCC CAG TA |
| | | G |
| 34 | GDA-501.2g: | MDFQVQIFSFLLISASVIMSRGDIQMTQSPSSLSASVGDRVTITC |
| | anti-Her2 | RASQDVNTAVAWYQQKPGKAPKLLIYSASFLYSGVPSRFSGSRS |
| | scFv- | GTDFTLTISSLQPEDFATYYCQQHYTTPPTFGQGTKVEIKRTGST |
| | CD8hinge+TM- | SGSGKPGSGEGSEVQLVESGGGLVQPGGSLRLSCAASGFNIKD |
| | 4-1BB- | TYIHWVRQAPGKGLEWVARIYPTNGYTRYADSVKGRFTISADT |
| | FCepsilonR | SKNTAYLQMNSLRAEDTAVYYCSRWGGDGFYAMDVWGQGT |
| | Full AA Seq | LVTVSSAATTTPAPRPPTPAPTIASQPLSLRPEACRPAAGGAVHT |
| | | RGLDFACDIYIWAPLAGTCGVLLLSLVITKRGRKKLLYIFKQPFM |
| | | RPVQTTQEEDGCSCRFPEEEEGGCELQVRKAAITSYEKSDGVYT |
| | | GLSTRNQETYETLKHEKPPQ |
| 35 | GDA-501.3g: | ATG GAT TTC CAG GTT CAG ATA TTC AGT TTT TTG CTC |
| | anti-Her2 | ATT TCC GCC AGT GTC ATC ATG AGC AGG GGC GAT ATT |
| | scFv- | CAG ATG ACC CAG TCT CCA AGT TCT CTT TCA GCG TCA |
| | CD28hinge- | GTA GGA GAT CGA GTA ACT ATA ACT TGC AGA GCT TCA |
| | TM-Cy- | CAG GAT GTC AAC ACG GCC GTA GCC TGG TAT CAA CAA |
| | FCepsilonR | AAA CCA GGG AAG GCG CCG AAA CTT CTC ATT TAC AGT |
| | Full Seq | GCA TCC TTC CTT TAC AGC GGA GTG CCT TCC CGC TTT |
| | codon | AGC GGG TCA CGC AGT GGT ACT GAC TTC ACG CTT ACA |
| | optimization | ATA TCT AGT CTC CAG CCT GAA GAT TTT GCA ACG TAC |
| | | TAC TGC CAA CAA CAC TAT ACT ACT CCG CCC ACA TTC |
| | | GGC CAG GGC ACA AAA GTA GAG ATA AAG CGC ACG GGT |
| | | AGC ACG TCA GGT TCA GGC AAA CCA GGT TCC GGA GAA |
| | | GGC AGT GAA GTT CAG CTG GTC GAA AGC GGG GGC GGA |
| | | CTC GTG CAA CCC GGC GGC AGT CTC AGA CTT TCA TGC |
| | | GCC GCG AGT GGT TTC AAT ATC AAA GAT ACC TAC ATA |
| | | CAC TGG GTT AGG CAG GCC CCA GGA AAA GGC CTC GAA |
| | | TGG GTT GCT CGA ATC TAC CCA ACC AAC GGA TAC ACA |
| | | AGG TAC GCA GAC TCT GTG AAA GGC AGA TTT ACG ATC |
| | | TCT GCA GAC ACG TCC AAG AAC ACC GCT TAC CTT CAA |
| | | ATG AAC TCA CTC CGG GCC GAA GAT ACA GCC GTA TAT |
| | | TAT TGC AGC CGA TGG GGA GGG GAC GGG TTC TAC GCA |
| | | ATG GAT GTA TGG GGT CAG GGC ACC TTG GTA ACA GTG |
| | | TCC TCT GCA ATC GAG GTC ATG TAC CCG CCC CCT TAC |
| | | CTG GAT AAT GAG AAA TCA AAT GGT ACC ATC ATT CAT |
| | | GTG AAG GGG AAG CAC CTT TGC CCA AGT CCG CTC TTT |
| | | CCA GGG CCC TCC AAG CCG TTT TGG GTC CTT GTT GTC |
| | | GTT GGT GGA GTG CTG GCT TGT TAC AGC TTG CTT GTA |
| | | ACG GTA GCT TTT ATA ATA TTC TGG GTG AGA TCT AAG |
| | | CGG TCT CGC CTC CTC CAC TCT GAC TAC ATG AAT ATG |
| | | ACT CCC CGC CGC CCG GGC CCA ACG CGC AAG CAT TAC |
| | | CAG CCG TAC GCA CCA CCT AGG GAC TTT GCC GCT TAT |
| | | CGG TCA CAA GTA AGA AAA GCA GCA ATT ACA TCC TAT |
| | | GAG AAG TCC GAT GGG GTT TAT ACA GGT CTC AGT ACA |
| | | AGG AAT CAA GAA ACG TAC GAG ACT TTG AAG CAC GAG |
| | | AAG CCA CCG CAG TAG |
| 36 | GDA-501.3g: | MDFQVQIFSFLLISASVIMSRGDIQMTQSPSSLSASVGDRVTITC |
| | anti-Her2 | RASQDVNTAVAWYQQKPGKAPKLLIYSASFLYSGVPSRFSGSRS |
| | scFv- | GTDFTLTISSLQPEDFATYYCQQHYTTPPTFGQGTKVEIKRTGST |
| | CD28hinge- | SGSGKPGSGEGSEVQLVESGGGLVQPGGSLRLSCAASGFNIKD |
| | TM-Cy- | TYIHWVRQAPGKGLEWVARIYPTNGYTRYADSVKGRFTISADT |
| | FCepsilonR AA | SKNTAYLQMNSLRAEDTAVYYCSRWGGDGFYAMDVWGQGT |
| | Full Seq | LVTVSSAIEVMYPPPYLDNEKSNGTIIHVKGKHLCPSPLFPGPSK |
| | | PFWVLVVVGGVLACYSLLVTVAFIIFWVRSKRSRLLHSDYMNM |
| | | TPRRPGPTRKHYQPYAPPRDFAAYRSQVRKAAITSYEKSDGVYT |
| | | GLSTRNQETYETLKHEKPPQ*X |
| 37 | GDA-501.4g: | ATG GAC TTT CAA GTA CAA ATT TTT AGC TTC CTC TTG |
| | anti-Her2 | ATC TCA GCA AGT GTA ATA ATG TCC CGA GGT GAC ATA |
| | scFv- | CAG ATG ACA CAA AGT CCC TCC TCA CTT AGC GCT TCT |
| | CD8hinge+TM- | GTG GGA GAC CGA GTA ACA ATA ACT TGC AGA GCG TCC |
| | 2B4- | CAA GAT GTT AAC ACA GCC GTA GCA TGG TAT CAG CAA |
| | FCepsilonR | AAG CCC GGT AAA GCG CCG AAA CTG CTC ATC TAT TCA |
| | Full Seq | GCG TCC TTC CTC TAT TCA GGG GTG CCC AGC AGA TTC |
| | codon | TCT GGT AGC CGC TCC GGG ACT GAC TTT ACT TTG ACT |
| | optimization | ATT AGT TCT CTC CAG CCG GAA GAC TTC GCC ACT TAC |
| | | TAT TGC CAA CAG CAC TAC ACA ACC CCC CCG ACT TTT |
| | | GGG CAG GGT ACT AAA GTT GAA ATC AAA CGC ACG GGC |
| | | TCC ACC AGT GGA TCC GGG AAA CCT GGA AGC GGC GAA |
| | | GGT TCC GAA GTT CAG CTC GTT GAA TCC GGG GGT GGC |
| | | CTT GTG CAA CCT GGA GGA TCC CTT CGG TTG AGT TGC |
| | | GCA GCC TCA GGA TTT AAC ATT AAA GAC ACG TAT ATC |
| | | CAT TGG GTG AGG CAG GCC CCA GGG AAG GGT TTG GAG |
| | | TGG GTT GCC AGA ATA TAC CCT ACA AAC GGT TAT ACC |
| | | CGC TAC GCG GAC AGC GTC AAA GGC CGG TTC ACT ATC |
| | | AGC GCG GAT ACA AGC AAG AAT ACT GCA TAT CTC CAG |
| | | ATG AAT TCA CTG CGG GCT GAA GAC ACA GCC GTT TAT |
| | | TAC TGC TCT CGA TGG GGA GGA GAT GGT TTC TAT GCG |
| | | ATG GAC GTC TGG GGC CAG GGG ACG CTT GTT ACA GTC |
| | | TCT TCA GCA ACA ACT ACT CCA GCC CCT CGA CCT CCA |
| | | ACG CCA GCA CCC ACC ATA GCT TCA CAA CCC CTT TCT |
| | | CTG CGC CCG GAG GCA TGT AGA CCA GCC GCC GGA GGA |
| | | GCG GTG CAC ACA CGG GGG CTC GAC TTT GCA TGC GAC |
| | | ATT TAT ATA TGG GCC CCT CTT GCA GGT ACC TGC GGT |
| | | GTT TTG TTG TTG TCA TTG GTC ATA ACA TGG AGA AGA |
| | | AAA CGA AAG GAA AAA CAG TCA GAG ACA TCT CCA AAG |
| | | GAG TTC CTC ACT ATC TAT GAG GAT GTG AAG GAT TTG |
| | | AAG ACG CGC AGA AAC CAT GAG CAG GAG CAA ACT TTC |
| | | CCC GGT GGT GGA TCT ACG ATT TAC TCC ATG ATT CAA |
| | | TCT CAA TCT TCC GCG CCT ACG TCC CAG GAA CCG GCA |
| | | TAT ACT CTT TAT TCT CTC ATT CAG CCT AGC CGG AAG |
| | | AGT GGC TCC AGA AAA CGC AAT CAT TCC CCG TCA TTT |
| | | AAC TCA ACC ATC TAT GAG GTG ATT GGT AAG TCT CAG |
| | | CCG AAA GCA CAA AAT CCC GCT AGG CTG AGT CGG AAA |
| | | GAG CTT GAG AAC TTC GAC GTG TAT AGT CAG GTT CGA |
| | | AAG GCA GCA ATC ACC AGC TAC GAA AAA AGC GAC GGT |
| | | GTC TAT ACT GGC TTG TCC ACT AGA AAC CAG GAA ACG |
| | | TAT GAA ACA CTT AAG CAT GAG AAA CCC CCA CAA TAG |
| 38 | GDA-501.4g: | MDFQVQIFSFLLISASVIMSRGDIQMTQSPSSLSASVGDRVTITC |
| | anti-Her2 | RASQDVNTAVAWYQQKPGKAPKLLIYSASFLYSGVPSRFSGSRS |
| | scFv- | GTDFTLTISSLQPEDFATYYCQQHYTTPPTFGQGTKVEIKRTGST |
| | CD8hinge+TM- | SGSGKPGSGEGSEVQLVESGGGLVQPGGSLRLSCAASGFNIKD |
| | 2B4- | TYIHWVRQAPGKGLEWVARIYPTNGYTRYADSVKGRFTISADT |
| | FCepsilonR | SKNTAYLQMNSLRAEDTAVYYCSRWGGDGFYAMDVWGQGT |
| | Full AA Seq | LVTVSSATTTPAPRPPTPAPTIASQPLSLRPEACRPAAGGAVHTR |
| | | GLDFACDIYIWAPLAGTCGVLLLSLVITWRRKRKEKQSETSPKEF |
| | | LTIYEDVKDLKTRRNHEQEQTFPGGGSTIYSMIQSQSSAPTSQE |
| | | PAYTLYSLIQPSRKSGSRKRNHSPSFNSTIYEVIGKSQPKAQNPA |
| | | RLSRKELENFDVYSQVRKAAITSYEKSDGVYTGLSTRNQETYETL |
| | | KHEKPPQ*X |
| 39 | CXCR4R334X | ATG GAG GGT ATT TCA ATT TAT ACC TCC GAC AAT TAC A |
| | DNA | CG GAG GAA ATG GGA AGT GGA GAT TAC GAC TCC ATG A |
| | sequence | AA GAG CCA TGC TTT AGG GAA GAG AAC GCC AAC TTT A |
| | codon | AT AAG ATC TTT CTC CCG ACA ATT TAT TCT ATT ATC TTC |
| | optimized | CTC ACC GGC ATT GTC GGA AAT GGT CTT GTT ATT CTT GT |
| | | T ATG GGC TAT CAA AAG AAA CTT CGA TCT ATG ACT GAT |
| | | AAG TAC AGG CTT CAC CTC AGT GTC GCC GAT CTG TTG T |
| | | TT GTA ATA ACT CTT CCG TTT TGG GCG GTA GAT GCC GTT |
| | | GCT AAC TGG TAC TTC GGT AAC TTC CTT TGT AAA GCT G |
| | | TA CAT GTC ATT TAC ACA GTT AAT CTG TAT AGC AGC GT |
| | | G CTG ATA CTG GCG TTT ATT TCA CTG GAC CGC TAC CTC |
| | | GCC ATC GTT CAC GCC ACG AAT AGT CAG CGA CCG CGG |
| | | AAG CTC TTG GCC GAG AAG GTT GTG TAC GTG GGC GTG |
| | | TGG ATT CCC GCT CTG CTC TTG ACA ATT CCC GAC TTC AT |
| | | A TTT GCC AAT GTG AGC GAA GCC GAT GAT CGC TAC ATC |
| | | TGT GAT AGA TTC TAT CCG AAT GAT TTG TGG GTA GTA G |
| | | TT TTT CAA TTC CAA CAC ATC ATG GTG GGC CTG ATC TTG |
| | | CCG GGA ATC GTA ATC CTG AGT TGT TAC TGC ATT ATT A |
| | | TC TCT AAG TTG TCC CAC TCA AAG GGC CAT CAG AAG CG |
| | | C AAG GCG TTG AAG ACG ACT GTT ATA CTC ATA TTG GCA |
| | | TTC TTT GCC TGT TGG CTG CCT TAC TAT ATT GGG ATA TC |
| | | T ATA GAT TCT TTC ATA CTC CTG GAG ATC ATA AAG CAG |
| | | GGA TGT GAA TTT GAA AAT ACT GTA CAT AAG TGG ATT T |
| | | CC ATA ACT GAA GCG CTT GCG TTC TTT CAT TGT TGT CTG |
| | | AAT CCA ATT CTC TAT GCG TTT CTG GGG GCA AAA TTT A |
| | | AA ACC TCA GCG CAA CAT GCA CTG ACC AGT GTA TCA CG |
| | | G GGC TCT AGC CTT AAA ATA CTT TCC AAA GGC AAG TAA |
| 40 | CXCR4R334X | MEGISIYTSDNYTEEMGSGDYDSMKEPCFREENANFNKIFLPTI |
| | amino acid | YSIIFLTGIVGNGLVILVMGYQKKLRSMTDKYRLHLSVADLLFVIT |
| | sequence | LPFWAVDAVANWYFGNFLCKAVHVIYTVNLYSSVLILAFISLDRY |
| | UniProtKB- | LAIVHATNSQRPRKLLAEKVVYVGVWIPALLLTIPDFIFANVSEA |
| | P61073 | DDRYICDRFYPNDLWVVVFQFQHIMVGLILPGIVILSCYCIIISKLS |
| | (CXCR4_HUMAN) | HSKGHQKRKALKTTVILILAFFACWLPYYIGISIDSFILLEIIKQGCE |
| | Feature key- | FENTVHKWISITEALAFFHCCLNPILYAFLGAKFKTSAQHALTSVS |
| | Natural | RGSSLKILSKGKX |
| | Variant | |
| | (VAR_081113), | |
| | 334-352 | |
| 41 | Anti-Her2 | GAT ATC CAG ATG ACC CAG TCC CCG AGC TCC CTG TCC |
| | Single-Chain | GCC TCT GTG GGC GAT AGG GTC ACC ATC ACC TGC CGT |
| | Variable | GCC AGT CAG GAT GTG AAT ACT GCT GTA GCC TGG TAT |
| | Fragment | CAA CAG AAA CCA GGA AAA GCT CCG AAA CTA CTG ATT |
| | (scFv)- DNA | TAC TCG GCA TCC TTC CTT TAT TCT GGA GTC CCT TCT |
| | sequence | CGC TTC TCT GGA TCT AGA TCT GGG ACG GAT TTC ACT |
| | | CTG ACC ATC AGC AGT CTG CAG CCG GAA GAC TTC GCA |
| | | ACT TAT TAC TGT CAG CAA CAT TAT ACT ACT CCT CCC |
| | | ACG TTC GGA CAG GGT ACC AAG GTG GAG ATC AAA CGC |
| | | ACT GGG TCT ACA TCT GGA TCT GGG AAG CCG GGT TCT |
| | | GGT GAG GGT TCT GAG GTT CAG CTG GTG GAG TCT GGC |
| | | GGT GGC CTG GTG CAG CCA GGG GGC TCA CTC CGT TTG |
| | | TCC TGT GCA GCT TCT GGC TTC AAC ATT AAA GAC ACC |
| | | TAT ATA CAC TGG GTG CGT CAG GCC CCG GGT AAG GGC |
| | | CTG GAA TGG GTT GCA AGG ATT TAT CCT ACG AAT GGT |
| | | TAT ACT AGA TAT GCC GAT AGC GTC AAG GGC CGT TTC |
| | | ACT ATA AGC GCA GAC ACA TCC AAA AAC ACA GCC TAC |
| | | CTG CAG ATG AAC AGC CTG CGT GCT GAG GAC ACT GCC |
| | | GTC TAT TAT TGT TCT AGA TGG GGA GGG GAC GGC TTC |
| | | TAT GCT ATG GAC GTG TGG GGT CAA GGA ACC CTG GTC |
| | | ACC GTC TCC TCG GCG GCC |
| 42 | Anti-Her2 | DIQMTQSPSSLSASVGDRVTITCRASQDVNTAVAWYQQKPGK |
| | Single-Chain | APKLLIYSASFLYSGVPSRFSGSRSGTDFTLTISSLQPEDFATYYCQ |
| | Variable | QHYTTPPTFGQGTKVEIKRTGSTSGSGKPGSGEGSEVQLVESG |
| | Fragment | GGLVQPGGSLRLSCAASGFNIKDTYIHWVRQAPGKGLEWVARI |
| | (scFv)-amino | YPTNGYTRYADSVKGRFTISADTSKNTAYLQMNSLRAEDTAVYY |
| | acid sequence | CSRWGGDGFYAMDVWGQGTLVTVSSAA |

1. A population of nucleated cells comprising a plurality of NK cells, wherein the population comprises at least 1.0×10⁶ nucleated cells, wherein at least about 70% of the cells in the population are viable and express an anti-Her2 chimeric antigen receptor (CAR), wherein:
at least about 85% of cells in the population are CD56+;
no more than about 0.5% of the cells in the population are CD3+;
no more than about 10% of the cells in the population are CD19+;
no more than about 10% of the cells in the population are CD14+;
at least about 60% of the cells in the population are CD62L+;
no more than about 20% of the cells in the population are LAG3+;
at least about 60% of the cells in the population are TRAIL+; and
at least about 60% of the cells in the population are DNAM1+,
and wherein the CAR comprises an anti-Her2 scFv.

2. (canceled)

3. The population of nucleated cells of claim 1, wherein the CAR comprises at least one of
(i) a hinge domain selected from CD28 and CD8;
(ii) a transmembrane domain selected from CD28, CD8, and NKG2D;
(iii) a co-stimulatory domain selected from CD28, 4-1BB, 2B4, CD3zetaR, OX40, Lsk, ICOS, DAP10, and Fc fragment of IgE receptor Ig;
(iv) an activation domain is selected from CD3ζ, FcR-γ, and Fc-epsilon-R; and
(v) a signal peptide.

4.-7. (canceled)

8. The population of nucleated cells of claim 1, wherein the CAR is selected from SEQ ID NO: 24, SEQ ID NO:26, SEQ ID NO: 28, SEQ ID NO: 30, SEQ ID NO: 32, SEQ ID NO: 34, SEQ ID NO: 36, and SEQ ID NO: 38.

9. The population of nucleated cells of claim 1, wherein at least about 75% of the CD56+ cells comprise any one of SEQ ID NO: 24, SEQ ID NO:26, SEQ ID NO: 28, SEQ ID NO: 30, SEQ ID NO: 32, SEQ ID NO: 34, SEQ ID NO: 36, or SEQ ID NO: 38.

10. A method of ex vivo producing natural killer (NK) cells of claim 1, the method comprising:
(a) expanding a population of NK cells by a method comprising:
(i) culturing said population of NK cells under conditions allowing for cell proliferation, wherein said conditions comprise providing an effective amount of nutrients, serum, IL-15 and nicotinamide; and
(ii) supplementing said population of NK cells with an effective amount of fresh nutrients, serum, IL-15 and nicotinamide 5-10 days following step (i) to produce expanded NK cells;
so as to obtain an ex vivo expanded population of NK cells, and
(b) upregulating expression of a CAR capable of binding HER2 in said ex vivo expanded population of NK cells, thereby producing the NK cells expressing the CAR capable of binding the HER2.

11. The method of claim 10, wherein said population of NK cells is derived from cord blood, peripheral blood, bone marrow, CD34+ cells or iPSCs.

12. The method of claim 10, wherein said population of NK cells is deprived of CD3⁺ cells.

13. The method of claim 10, wherein said population of NK cells comprises CD3⁻CD56⁺ cells.

14. The method of claim 10, wherein said effective amount of said nicotinamide comprises an amount between 1.0 mM to 10 mM.

15. The method of claim 10, wherein:
(i) said expanding said population of NK cells is affected in the presence of feeder cells or a feeder layer, optionally
(ii) wherein said feeder cells comprise irradiated cells, optionally
(iii) wherein said feeder cells comprise T cells or PBMCs, optionally
(iv) wherein said feeder cells comprise an CD3 agonist and, optionally
(v) wherein said CD3 agonist is OKT3.

16.-18. (canceled)

19. The method of claim 10, wherein said expanding said population of NK cells is affected for 14-16 days.

20. The method of claim 10, wherein said upregulating expression of said CAR or said tg-TCR is affected on day 12-14 from initiation of culture.

21. The method of claim 10, wherein said upregulating expression of said CAR or said tg-TCR is affected with naked nucleic acid or with a vector.

22. The method of claim 10, wherein said upregulating expression of said CAR or said tg-TCR is affected by mRNA electroporation and, optionally, wherein said CAR or said tg-TCR is transiently expressed.

23. An isolated population of NK cells of claim 1 obtainable according to a method comprising:
(a) expanding a population of NK cells by a method comprising:
(i) culturing said population of NK cells under conditions allowing for cell proliferation, wherein said conditions comprise providing an effective amount of nutrients, serum, IL-15 and nicotinamide; and
(ii) supplementing said population of NK cells with an effective amount of fresh nutrients, serum, IL-15 and nicotinamide 5-10 days following step (i) to produce expanded NK cells;
so as to obtain an ex vivo expanded population of NK cells, and
(b) upregulating expression of a CAR capable of binding HER2 in said ex vivo expanded population of NK cells, thereby producing the NK cells expressing the CAR capable of binding the HER2.

24. A pharmaceutical composition comprising the isolated population of NK cells of claim 1 and a pharmaceutically active carrier.

25. A method of treating a disease associated with expression of HER2 in a subject in need thereof, the method comprising administering to the subject a therapeutically effective amount of the pharmaceutical composition of claim 24, thereby treating the subject.

26. (canceled)

27. The method of claim 25, wherein the disease is a malignant disease.

28. The method of claim 27, wherein said malignant disease is a solid tumor or tumor metastasis.

29. The method of claim 28, wherein said malignant disease is selected from the group consisting of a breast cancer, a gastric cancer, a gastroesophageal cancer, an oesophageal cancer, an ovarian cancer, an endometrial cancer, a lung cancer, an urothelial cancer and a bladder cancer.

30. The method of claim 25, wherein the subject is a human subject.
